(19) United States
(12) Patent Application Publication
(10) Pub. No.: US 2026/0137466 A1
(43) Pub. Date: May 21, 2026

(54) DRIVING ELEMENT EQUIPPED WITH ELASTIC JOINT AND SURGICAL ROBOT SYSTEM COMPRISING THE SAME

(71) Applicant: LivsMed Inc., Seongnam-si (KR)

(72) Inventors: Young Wan KIM, Seongnam-si (KR); So Yeon KIM, Yongin-si (KR); Jin Hyuk YOON, Seongnam-si (KR)

(73) Assignee: LivsMed Inc., Seongnam-si (KR)

(21) Appl. No.: 19/368,402

(22) Filed: Oct. 24, 2025

(30) Foreign Application Priority Data
Nov. 19, 2024 (KR) 10-2024-0165086

(51) Int. Cl. A61B 34/37; A61B 34/00; A61B 34/30

(52) U.S. Cl. CPC A61B 34/37; A61B 34/71; A61B 2034/302

(57) ABSTRACT

Provided is a surgical robot system including at least one driving element and a processor configured to control the driving element. The driving element includes: a first member; a second member configured to change a relative position of the second member with respect to a first member; and at least one joint member configured to provide a rotational coupling between the first member and the second member, wherein the at least one joint member is an elastic joint member configured to allow twisting between the first member and the second member, which are targets of rotational coupling due to an external force.

### CROSS-REFERENCE TO RELATED APPLICATION

This application claims the benefit of and priority to Korean Patent Application No. 10-2024-0165086, filed on Nov. 19, 2024, the entire disclosure(s) of which is hereby incorporated herein by reference in its entirety.

### BACKGROUND

### 1. Field

The present disclosure relates to a surgical robot, and more specifically, but not limitedly, to a driving element that allows twisting between links due to an external force and enables mutual collision, and a surgical robot system including the same.

### 2. Description of the Related Art

In medical terms, surgery refers to the treatment of a disease by using medical devices to cut, slit, or manipulate skin, a mucous membrane, or other tissue. In particular, open surgery of cutting and opening the skin of a surgical site to treat, reshape, or remove organs therein causes bleeding, side effects, pain to a patient, and scars. Accordingly, recently, surgery using a robot or surgery performed by inserting only a medical device, for example, a laparoscope, a surgical instrument, a microsurgical microscope, or the like, in the body by forming a predetermined hole in the skin, has been spotlighted as an alternative.

Herein, a surgical robot refers to a robot that has a function of replacing a surgical action performed by a surgeon. The surgical robot may operate more accurately and precisely as compared with a human and enable remote surgery.

A surgical robot system is generally composed of a master robot and a slave robot. When a surgical operator manipulates a control lever (for example, a handle) provided on the master robot, a surgical instrument coupled to or held by a robot arm on the slave robot is manipulated to perform surgery.

When a surgery is performed using the surgical robot system, the robot arm performs the surgery under the control of an operating surgeon. In this connection, the surgical instrument operates inside the body of a patient, and a plurality of structures including the robot arm for controlling the same from outside the body of the patient are disposed. The surgical instrument used for laparoscopic surgery is inserted into the body of the patient through at least one port formed in the body. In order to prevent damage to the body of the patient, the surgical robot is required to operate while maintaining this port as a remote center of motion. In other words, even when the robot arm and/or the surgical instrument of the surgical robot operates, the point corresponding to the port formed in the body of the patient needs to be controlled in a fixed state. However, when the robot arm and/or the surgical instrument of the surgical robot collides with a specific object, the remote center of motion may be deviated due to the collision, causing damage to the body of the patient.

The aforementioned background technology corresponds to technical information that has been possessed by the present inventor(s) in order to derive the present disclosure or which has been acquired in the process of deriving the present disclosure, and may not necessarily be regarded as well-known technology which had been known to the public prior to the filing of the present disclosure.

### SUMMARY

An exemplary aspect of the present disclosure is directed to providing a driving element that allows twisting between links due to an external force and enables mutual collision, and a surgical robot system including the same. In addition, an aspect of the present disclosure is directed to providing a computer-readable recording medium recording a program for executing the method on a computer.

The aspects of the present disclosure are not limited to those mentioned above, and other aspects and benefits not mentioned may be understood from the following description and may be more clearly understood by the embodiments of the present disclosure. In addition, the aspects and benefits to be solved by the present disclosure may be realized by the means indicated in the scope of claims and combinations thereof.

A surgical robot system according to an embodiment of the present disclosure includes at least one driving element and a processor configured to control the driving element, wherein the driving element includes: a first member; a second member configured to change a relative position of the second member with respect to a first member; and at least one joint member configured to provide a rotational coupling between the first member and the second member. Herein, the at least one joint member may be an elastic joint member configured to allow twisting between the first member and the second member, which are targets of rotational coupling due to an external force.

A surgical robot system according to an embodiment of the present disclosure includes at least one surgical robot arm and a processor configured to control the surgical robot arm, wherein the robot arm includes: a base link extending from a main body of the at least one surgical robot; an instrument mounting link on which a surgical instrument to be inserted into a patient through a fixed remote center of motion (RCM) is mounted; at least one intermediate link interposed between the base link and the instrument mounting link; at least one yaw joint configured to provide a yaw direction rotational coupling of the robot arm between any links among the base link, the instrument mounting link and the at least one intermediate link; and at least one pitch joint providing a pitch direction rotational coupling of the robot arm between links among the base link, the instrument mounting link and the at least one intermediate link, wherein at least one of the at least one yaw joint and the at least one pitch joint may be an elastic joint configured to allow twisting between links, which are the targets of rotational coupling due to the external force.

According to an aspect, a yaw direction rotation axis of the robot arm by the yaw joint may be configured to intersect the RCM.

According to an aspect, the robot arm may include a plurality of pitch joints, which include the at least one pitch joint, and rotations of the plurality of pitch joints may be configured to be interlinked with each other.

According to an aspect, the rotations of the plurality of pitch joints may be configured to be interlinked with each other by a fixed belt that interlinks the rotations of at least two pitch joints.

According to an aspect, the twisting by the elastic joint may be configured to be performed along a rotational direction by the at least one yaw joint or the at least one pitch joint.

According to an aspect, the robot arm may be configured such that, by providing the elastic joint, when an external force is applied to the robot arm, twisting occurs in at least one of the yaw joint or the pitch joint along the rotational direction, so that the surgical instrument does not deviate from the RCM.

According to an aspect, the elastic joint may include: a motor coupled to a first link that is a target of the rotational coupling of the elastic joint; and an elastic member whose first end is coupled to the motor and whose second end is coupled to a second link that is the target of the rotational coupling of the elastic joint, thereby providing twisting in the rotational direction of the rotational coupling of the elastic joint.

According to an aspect, the elastic member may be a coil spring that surrounds a rotation axis of the rotational coupling provided by the elastic joint.

According to an aspect, the yaw joint may include a first elastic member having a first elastic modulus, and the pitch joint may include a second elastic member having a second elastic modulus, wherein the second elastic modulus may be configured to be greater than the first elastic modulus.

According to an aspect, the elastic joint may further include a first encoder configured to detect rotational position of the motor; and a second encoder configured to detect a rotational position of the second link.

According to an aspect, the processor may be further configured to determine a rotation amount of the elastic joint due to the external force based on information on the rotational position of the motor and information on the rotational position of the second link.

According to an aspect, the processor may be further configured to determine a magnitude of an external force applied to the robot arm at a first time point based on information on the rotational position of the motor, information on the rotational position of the second link, and an elastic modulus of the elastic member.

According to an aspect, the magnitude of the external force may include information on a torque applied to the elastic joint.

According to an aspect, the processor may be further configured to control a motor of the elastic joint so that the elastic joint complies with the external force in response to a determination that the determined magnitude of the external force at the first time point is greater than or equal to a predetermined first threshold value.

According to an aspect, the processor may be further configured to stop controlling of the robot arm in response to a determination that the determined magnitude of the external force at the first time point is greater than or equal to a predetermined second threshold value.

According to an aspect, the processor may be further configured to output warning information through an output interface provided in the surgical robot system in response to a determination that the determined magnitude of the external force at the first time point is greater than or equal to a predetermined third threshold value.

According to an aspect, the processor may be further configured to stop controlling of the surgical instrument and maintain controlling of the robot arm in response to a determination that the determined magnitude of the external force at the first time point is greater than or equal to a predetermined fourth threshold value and less than a predetermined second threshold value.

According to an aspect, the processor may be further configured to determine the magnitude of the external force applied to the robot arm at a second time point subsequent to the first time point, and to resume controlling of the surgical instrument in response to a determination that the magnitude of the external force at the second time point is less than the fourth threshold value.

According to an aspect, the driving element may include a slide driving unit configured to control linear motion of an instrument mounting unit in a patient insertion direction; the first member may include a slide motor provided in the slide driving unit; the second member may include a slide shaft provided in the slide driving unit; and the joint member may include an elastic member disposed between the slide motor and the slide shaft.

According to an aspect, the driving element may include a driving unit of a surgical instrument and an instrument mounting unit; the first member may include at least one pulley provided in the driving unit of the surgical instrument; the second member may include an instrument driving motor provided in the instrument mounting unit; and the joint member may include an elastic member disposed between the at least one pulley and the instrument driving motor.

A robot arm according to another embodiment of the present disclosure is a surgical robot arm on which a surgical instrument is mounted, and includes: a base link extending from a main body of a surgical robot; an instrument mounting link on which a surgical instrument to be inserted into a patient through a fixed remote center of motion (RCM) is mounted; at least one intermediate link interposed between the base link and the instrument mounting link; at least one yaw joint providing a yaw direction rotational coupling of the robot arm between two links among the base link, the instrument mounting link and the at least one intermediate link; and at least one pitch joint providing a pitch direction rotational coupling of the robot arm between two links among the base link, the instrument mounting link and the at least one intermediate link, wherein at least one of the plurality of joints including the yaw joint or the pitch joint may be an elastic joint that allows twisting between the links that are targets of rotational coupling due to an external force.

In addition, another method for implementing the present disclosure, another system, and a computer-readable recording medium storing a computer program for executing the method may be further provided.

Other aspects, features, and advantages in addition to those described above will become apparent from the following drawings, claims, and detailed description of the present disclosure.

By configuring at least the joint of the robot arm equipped in the surgical robot as an elastic joint that allows twisting between links due to an external force, even when the robot arm and/or the surgical instrument of the surgical robot collides with a specific object, the RCM set at the port of a patient body can be prevented from being distorted. Accordingly, it is possible to design a safe surgical robot system that does not cause damage to the patient body even when a collision occurs in the surgical robot.

In addition, it is possible to measure the rotational angle due to an external force or the magnitude of the external force based on the rotational position of the links on both sides coupled by the elastic joint and the characteristics of the elastic joint. Accordingly, it is possible to measure an external force such as a torque acting on the surgical robot without having an expensive torque sensor, and to control the surgical robot in compliance with the extent of the external force, to stop the control, or to output warning information to a user.

The benefits of the present disclosure are not limited to those mentioned above, and other benefits not mentioned may be clearly understood by those skilled in the art from the following description.

### BRIEF DESCRIPTION OF THE DRAWINGS

FIG. 1 is a diagram for explaining an example of a system for driving a surgical instrument according to an embodiment.

FIG. 2A is a configuration diagram illustrating an example of a user terminal according to an embodiment.

FIG. 2B is a configuration diagram illustrating an example of a server according to an embodiment.

FIG. 3 is a conceptual diagram illustrating a surgical robot system according to an embodiment.

FIG. 4 is a block diagram illustrating the internal configuration of the surgical robot system of FIG. 3.

FIG. 5 is a perspective view of a slave robot of the surgical robot system of FIG. 3 and a surgical instrument mounted thereon.

FIG. 6 is a perspective view of a modular slave robot and a surgical instrument mounted thereon according to an aspect of the surgical robot system of FIG. 3.

FIG. 7 is a diagram illustrating a state in which the instrument case is removed from FIG. 6.

FIG. 8 is a perspective view of a modular slave robot and a laparoscopic surgical camera mounted thereon according to another aspect of the surgical robot system of FIG. 3.

FIG. 9 is a diagram illustrating a state in which the surgical instrument is removed from the slave robot of FIG. 6.

FIG. 10 is a perspective view of another example of a modular slave robot and a surgical instrument mounted thereon of a surgical robot system according to an embodiment.

FIG. 11 is a perspective view of a surgical instrument according to an embodiment of the present disclosure.

FIGS. 12 and 13 are perspective views of an end tool of the surgical instrument of FIG. 11.

FIGS. 14A to 14B is a plan view of the end tool of the surgical instrument of FIG. 11.

FIGS. 15 and 16 are perspective views of a driving part of the surgical instrument of FIG. 11

FIG. 17 is a plan view of the driving part of the surgical instrument of FIG. 11.

FIG. 18 is a rear view of the driving part of the surgical instrument of FIG. 11.

FIG. 19 is a side view of the driving part of the surgical instrument of FIG. 11.

FIG. 20 is a diagram illustrating the configuration of pulleys and wires of the surgical instrument illustrated in FIG. 11, in detail for the configuration related to a first jaw.

FIG. 21 is a diagram illustrating the configuration of pulleys and wires of the surgical instrument illustrated in FIG. 11, in detail for the configuration related to a second jaw.

FIGS. 22A to 23C are diagrams illustrating a pitch motion of the surgical instrument illustrated in FIG. 11.

FIGS. 24A to 25B are diagrams illustrating a yaw motion of the surgical instrument illustrated in FIG. 11.

FIG. 26 is a perspective view of a surgical robot system having an elastic joint according to an embodiment of the present disclosure.

FIG. 27 is a side view of a yaw rotation axis of a robot arm of the surgical robot system of FIG. 26.

FIG. 28 is a side view illustrating rotational linkage of pitch joints of a robot arm of the surgical robot system of FIG. 26.

FIG. 29 is a side view illustrating maintenance of remote center of motion according to pitch control of the robot arm of FIG. 28.

FIG. 30 is an exemplary view of an elastic joint according to an aspect of the present disclosure.

FIG. 31 is an exemplary flowchart of a rotation amount determination procedure for a robot arm according to an embodiment of the present disclosure.

FIG. 32 is an exemplary flowchart of an external force magnitude determination procedure for a robot arm according to an embodiment of the present disclosure.

FIG. 33 is an exemplary flowchart of an external force compliance control procedure for a robot arm according to an embodiment of the present disclosure.

FIG. 34 is an exemplary flowchart of a control interruption procedure for a robot arm according to an embodiment of the present disclosure.

FIG. 35 is an exemplary flowchart of a warning information output procedure for a robot arm according to an embodiment of the present disclosure.

FIG. 36 is an exemplary flowchart of a control interruption and resumption procedure for a robot arm and/or a surgical instrument according to an embodiment of the present disclosure.

FIG. 37 is a perspective view of a slide driving unit having an elastic joint member according to an embodiment of the present disclosure.

FIG. 38 is a perspective view of a driving unit and/or an instrument mounting unit of a surgical instrument having an elastic joint member according to an embodiment of the present disclosure.

### DETAILED DESCRIPTION

Hereinafter, various embodiments of the present disclosure are described in conjunction with the accompanying drawings. Various embodiments of the present disclosure may make various changes and have various embodiments, and specific embodiments are illustrated in the drawings and related detailed descriptions are described. However, this is not intended to limit the various embodiments of the present disclosure to specific embodiments, and should be understood to include all changes and/or equivalents or substitutes included in the spirit and technical scope of the various embodiments of the present disclosure. In connection with the description of the drawings, similar reference numerals have been used for similar components.

Expressions such as “comprise” or “may comprise” that may be used in various embodiments of the present disclosure indicate the presence of the corresponding function, operation, or component disclosed, and do not limit one or more additional functions, operations, or components. In addition, in various embodiments of the present disclosure, terms such as “comprise” or “have” are used to specify the presence of stated features, integers, steps, operations, components, parts, or a combination thereof, but do not preclude the presence or addition of one or more other features, integers, steps, operations, components, parts, or combinations thereof.

In various embodiments of the present disclosure, the expression such as “or” includes any and all combinations of words listed together. For example, “A or B” may include A, B, or both A and B.

Although the expressions such as “first,” “second,” etc. used in various embodiments of the present disclosure may modify various components of the various embodiments, but do not limit the components. For example, the expressions do not limit the order and/or importance of corresponding components. These expressions may be used to distinguish one component from the other components. For example, a first user device and a second user device are both user devices and represent different user devices. For example, a first component may be referred to as a second component without departing from the scope of right of various embodiments of the present disclosure, and similarly, the second component may also be referred to as the first component.

In an embodiment of the present disclosure, terms such as “module,” “unit,” or “part” are used to refer to components that perform at least one function or operation, and these components may be implemented as hardware or software, or as a combination of hardware and software. In addition, a plurality of “modules,” “units,” “parts,” etc. may be integrated into at least one module or chip and implemented with at least one processor, except in the cases where each thereof needs to be implemented with individual specific hardware.

Terms used in various embodiments of the present disclosure are merely used to describe specific embodiments and are not intended to limit the various embodiments of the present disclosure. A singular expression includes a plural expression, unless the context clearly states otherwise.

Unless otherwise defined, all terms including technical and scientific terms used herein have the same meaning as commonly understood by those having ordinary skill in the art to which various embodiments of the present disclosure pertains.

It will be further understood that terms, such as those defined in commonly used dictionaries, should be interpreted as having a meaning that is consistent with their meaning in the context of the relevant art and will not be interpreted in an idealized or overly formal sense unless expressly so defined in various embodiments of the present disclosure.

Hereinafter, various embodiments of the present disclosure will be described in detail using the accompanying drawings.

Laparoscopic surgery refers to a surgery performed by forming a hole in the abdominal cavity of a patient, inserting a narrow and long tube through the hole, and using surgical instruments connected to the end. The surgical instrument may be, for example, an articulated instrument.

In this connection, when a passive surgical instrument is used, the surgical instrument and a control unit operated by a user move symmetrically with respect to a hole in the abdominal cavity, so that more than a certain period of practice is needed until a user becomes familiar with the control. In addition, since the surgical instruments may not be checked with the naked eye, the surgical instruments need to be manipulated while a surgical operator watches the camera images acquired by inserting an endoscopic camera into the abdominal cavity.

This situation is the same even when laparoscopic surgery is performed using a surgical robot system, but there is a benefit of being intuitively controlled compared to manual surgical instruments. As will be described later in the description, the surgical robot system according to an embodiment includes a master robot and a slave robot. The slave robot may be referred to as a surgical robot or surgical instrument, and may refer to a configuration that performs surgery by acting directly on a patient. The master robot may be referred to as a master device or a user input interface, and may refer to a configuration for receiving a user manipulation to control the slave robot.

This type of surgical robot system is mounted with articulated instruments and separates the portion that performs surgery (for example, surgical robot) and the portion that a user manipulates (for example, the master device), and thus intuitive control is possible compared to manual surgical instruments. In other words, the surgical robot system is capable of controlling operations so that surgical instruments may be intuitively controlled by matching the movements of a user with the movements on the laparoscopic camera screen.

### Surgical Robot System Driving

Hereinafter, a method and apparatus for driving a surgical instrument according to embodiments of the present disclosure will be described in more detail with reference to the drawings.

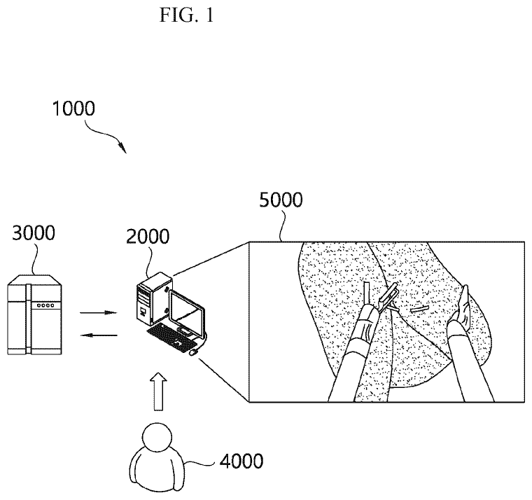

FIG. 1 is a diagram for explaining an example of a system for driving a surgical instrument according to an embodiment.

Referring to FIG. 1, a system 1000 includes a user terminal 2000 and a server 3000. For example, the user terminal 2000 and the server 3000 may be connected to each other through a wired or wireless communication method to transmit and/or receive data to and/or from each other.

For convenience of explanation, although FIG. 1 illustrates that the system 1000 includes the user terminal 2000 and the server 3000, an embodiment of the present disclosure is not limited thereto. For example, other external devices (not shown) may be included in the system 1000, and operations of the user terminal 2000 and the server 3000 to be described below may be implemented by a single device (for example, the user terminal 2000 or the server 3000) or a plurality of devices.

The user terminal 2000 may be a computing apparatus that is provided with a display apparatus and a device (for example, a keyboard, a mouse, or the like) for receiving a user input, and includes a memory and a processor. For example, the display apparatus may be implemented as a touch screen to receive user input. For example, the user terminal 2000 may correspond to a notebook PC, a desktop PC, a laptop, a tablet computer, a smartphone, or the like, but is not limited thereto.

The server 3000 may be an apparatus that communicates with an external device (not shown) including the user terminal 2000. As an example, the server 3000 may be an apparatus that stores various types of data.

Alternatively, the server 3000 may be a computing apparatus including a memory and a processor, and having its own computing capability. For example, the server 3000 may perform at least some of operations of the user terminal 2000 to be described below with reference to the drawings. For example, the server 3000 may also be a cloud server, but is not limited thereto.

According to an aspect, the user terminal 2000 may drive the surgical instrument. In this description, the method for driving the surgical instrument below may be described as being performed by a computing device. The computing device may be, for example, the user terminal 2000 or the server 3000, but is not limited thereto. Any single or plural computing devices including a processor may configure a computing device. Hereinafter, for convenience of explanation, the control procedure of the surgical instrument by the user terminal 2000 may be described, but this is only for explanation, and the method of controlling the surgical instrument according to embodiments of the present disclosure may be performed by any computing device.

Herein, the application of FIG. 1 may be a software program installed for the purpose of activities to drive the surgical robot system of a user 4000. For example, through the application, the user 4000 may generate manipulation information based on the user input to control the surgical robot system.

The user terminal 2000 may output an image 5000 representing the operation of the surgical instrument driven based on the operation of the user 4000. For example, the user terminal 2000 may generate manipulation information based on an amount of change in the reference posture of the user input interface for the user 4000 to control the surgical robot system. Then, the user terminal 2000 may decide the target posture of the surgical instrument corresponding to the manipulation information, and decide the target state information for the driving element. Subsequently, the user terminal 2000 may drive the driving element according to the decided target state information and output the image 5000 representing the operation of the surgical instrument driven in this way. The user 4000 may intuitively understand the operation of the surgical instrument according to the operation of the user through the image 5000 representing the operation of the surgical instrument and manipulate the surgical robot system more accurately.

As described above, at least some of the operations of the user terminal 2000 described below with reference to the drawings may be performed by the server 3000. For example, the server 3000 may perform various activities for controlling the surgical robot system. Alternatively, at least some of these activities may be performed by the server 3000, and at least some thereof may be performed by the user terminal 2000.

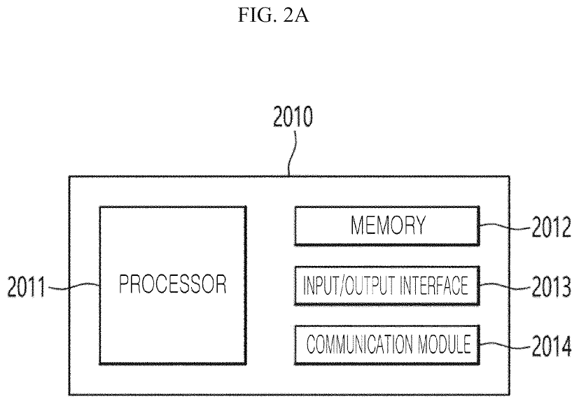

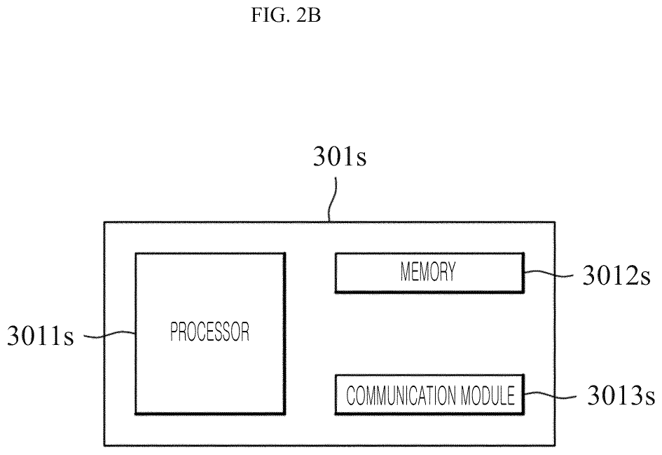

FIG. 2A is a configuration diagram illustrating an example of a user terminal according to an embodiment.

Referring to FIG. 2A, a user terminal 2010 includes a processor 2011, a memory 2012, an input/output interface 2013, and a communication module 2014. For convenience of explanation, FIG. 2A illustrates only components related to an embodiment of the present disclosure. Accordingly, the user terminal 2010 may further include other general-purpose components, in addition to the components illustrated in FIG. 2A. In addition, it is obvious to those skilled in the technical field to which the present disclosure pertains that the processor 2011, the memory 2012, the input/output interface 2013, and the communication module 2014 illustrated in FIG. 2A may also be implemented as independent devices.

The processor 2011 may process commands of a computer program by performing basic arithmetic, logic, and input/output operations. Herein, the commands may be provided from the memory 2012 or an external device (for example, the server 3000, etc.). In addition, the processor 2011 may control the overall operation of other components included in the user terminal 2010.

First, the processor 2011 generates manipulation information regarding the operations of a user to drive the surgical robot system. For example, the processor 2011 may generate manipulation information regarding the operation of the user based on a member that allows the position and function of the surgical instrument to be manipulated by the operation of the user.

The member for manipulating the position and function of the surgical instrument by the operation of a user may be formed in the form of a handle-shaped manipulation member, but is not limited thereto and may be modified and implemented in various shapes to achieve the same purpose. For example, some may be formed in the shape of a handle, and the others may be formed in a different shape, such as a clutch button. In addition, a finger insertion tube may be formed so as to allow the finger(s) of a surgical operator to be inserted therethrough and fixed to facilitate manipulation of a surgical instrument. Hereinafter, in this description, a member that allows manipulation by the operation of the user may also be referred to as the user input interface.

Herein, before the first manipulation of a user of the user input interface, the processor 2011 may update the reference posture of the user input interface with the posture information before manipulation of the user input interface. Since the driving of the surgical instrument by the user may be performed based on the degree to which the user input interface has changed by the user. Hence, by initializing the reference posture of the user input interface to the state before the manipulation before the user performs the first manipulation, the difference between the state of the user input interface after user manipulation and the state of the user input interface before user manipulation, in other words, an amount of change in the user input interface, may be decided.

The processor 2011 may generate manipulation information based on an amount of change in the reference posture of the user input interface. The manipulation information refers to information representing the intuitive operation of a user to manipulate the position and function of the surgical instrument. More specifically, but non-limitingly, the manipulation information may include position information and orientation information on a physical coordinate system of a member that allows a user to manipulate the position and function of the surgical instrument. As an example, the manipulation information may include a transformation matrix representing linear and rotational movement in a homogeneous coordinate system. The transformation matrix may be a homogeneous transformation matrix and may include rotation matrix information and translation vector information. As another example, the manipulation information may include position information and orientation information on a physical coordinate system expressed according to an expression method such as a screw. However, the examples of manipulation information are not limited to the above. The manipulation information may be decided based on an amount of change in the reference posture of the user input interface. Herein, the manipulation information may represent an amount of change with respect to the reference posture, and the reference posture may represent the degree of change of the user input interface with respect to the origin. However, the reference posture and manipulation information may be expressed, for example, by a homogeneous transformation matrix or a screw method as described above.

The processor 2011 may generate manipulation information based on a member that allows a user to manipulate the position and function of the surgical instrument, for example, position information and orientation information of the user input interface. For example, the processor 2011 may generate manipulation information using the difference between the initial position information and initial orientation information of the member that allows the user to manipulate the position and function of the surgical instrument, and the position information and orientation information after the operation of the user of the aforementioned member. According to an aspect, the processor 2011 may generate manipulation information based on an amount of change from the reference posture of the user input interface according to the manipulation of the user.

In addition, based on the manipulation information, the processor 2011 may decide the target posture of the surgical instrument corresponding to the manipulation information. For example, the processor 2011 may decide the target posture of the surgical instrument based on the manipulation information. According to an aspect, the processor 2011 may be configured to decide the target posture based on the correspondence relationship between a predetermined movement of the user input interface and the movement of the surgical instrument.

The processor 2011 may be implemented as an array of a plurality of logic gates, or may be implemented as a combination of a general-purpose microprocessor and a memory in which a program executable in the microprocessor is stored. For example, the processor 2011 may include a general-purpose processor, a central processing unit (CPU), a microprocessor, a digital signal processor (DSP), a controller, a microcontroller, a state machine, and the like. In some circumstances, the processor 110 may include an application-specific integrated circuit (ASIC), a programmable logic device (PLD), a field programmable gate array (FPGA), or the like. For example, the processor 2011 may refer to a combination of processing devices, such as a combination of a digital signal processor (DSP) and a microprocessor, a combination of a plurality of microprocessors, a combination of one or more microprocessors coupled with a digital signal processor (DSP) core, or a combination of any other configurations.

The memory 2012 may include any non-transitory computer-readable recording medium. In an embodiment, the memory 2012 may include a permanent mass storage device such as a random access memory (RAM), a read only memory (ROM), a disk drive, a solid state drive (SSD), a flash memory, etc. In another embodiment, a permanent mass storage device such as a ROM, SSD, a flash memory, a disk drive, etc. may be a separate permanent storage device which is distinguishable from the memory. In addition, an operating system (OS) and at least one program code (for example, a code for the processor 2011 to perform an operation to be described later with reference to the drawings) may be stored in the memory 2012.

These software components may be loaded from a computer-readable recording medium separate from the memory 2012. The separate computer-readable recording medium may be a recording medium that may be directly connected to the user terminal 2010, for example, a computer-readable recording medium, such as a floppy drive, a disk, a tape, a DVD/CD-ROM drive, a memory card, or the like. In addition, the software components may be loaded into the memory 2012 through the communication module 2014 instead of a computer-readable recording medium. For example, at least one program may be loaded into the memory 2012 based on a computer program (for example, a computer program for performing, by the processor 2011, an operation to be described later with reference to the drawings) installed by the files provided through the communication module 2014 by developers or a computer file distribution system that distributes the installation files of applications.

The input/output interface 2013 may be a member for an interface with a device (for example, a keyboard, a mouse, etc.) for input or output, the member being connected to the user terminal 2010 or being included in the user terminal 2010. The input/output interface 2013 may be configured separately from the processor 2011, without being limited thereto, and the input/output interface 2013 may be configured to be included in the processor 2011.

The communication module 2014 may provide a configuration or function for the server 3000 and the user terminal 2010 to communicate with each other through a network. In addition, the communication module 2014 may provide a configuration or function for the user terminal 2010 to communicate with another external device. For example, a control signal, a command, data, etc. provided according to the control of the processor 2011, may be transmitted to the server 3000 and/or an external device through the communication module 2014 and the network.

Although not illustrated in FIG. 2A, the user terminal 2010 may further include a display apparatus. For example, the display apparatus may be implemented as a touch screen. Alternatively, the user terminal 2010 may be connected to an independent display apparatus through a wired or wireless communication method to transmit and/or receive data to and/or from each other. For example, a video or image of driving the surgical instrument using driving information may be provided through the display apparatus.

FIG. 2B is a configuration diagram illustrating an example of a server according to an embodiment.

Referring to FIG. 2B, the server 301s includes a processor 3011s, a memory 3012s, and a communication module 3013s. For convenience of explanation, FIG. 2B illustrates only components related to an embodiment of the present disclosure. Accordingly, the server 301s may further include other general-purpose components, in addition to the components illustrated in FIG. 2B. In addition, it is obvious to those skilled in the technical field to which the present disclosure pertains that the processor 3011s, the memory 3012s, and the communication module 3013s illustrated in FIG. 2B may also be implemented as independent devices.

The processor 3011s may perform various activities for controlling the surgical robot system. In other words, at least one of the operations of the processor 2011 described above with reference to FIG. 2A may be performed by the processor 3011s. In this connection, the user terminal 2010 may output information transmitted from the server 301s through the display apparatus.

Since the implementation example of the processor 3011s is the same as the implementation example of the processor 2011 described above with reference to FIG. 2A, the detailed description thereof is omitted.

The memory 3012s may store various pieces of data, such as data necessary for the operation of the processor 3011s and data generated according to the operation of the processor 3011s. Additionally, an operating system (OS) and at least one program (for example, a program necessary for the processor 3011s to operate, etc.) may be stored in the memory 3012s.

Since the implementation example of the memory 3012s is the same as the implementation example of the memory 2012 described above with reference to FIG. 2A, the detailed description thereof will be omitted.

The communication module 3013s may provide a configuration or function for the server 301s and the user terminal 2010 to communicate with each other through a network. Additionally, the communication module 2014 may provide a configuration or function for the server 301s to communicate with other external devices. For example, control signals, commands, data, etc. provided under control of the processor 3011s may be transmitted to the user terminal 2010 and/or an external device through the communication module 3013s and a network.

### Surgical Robot System Configuration

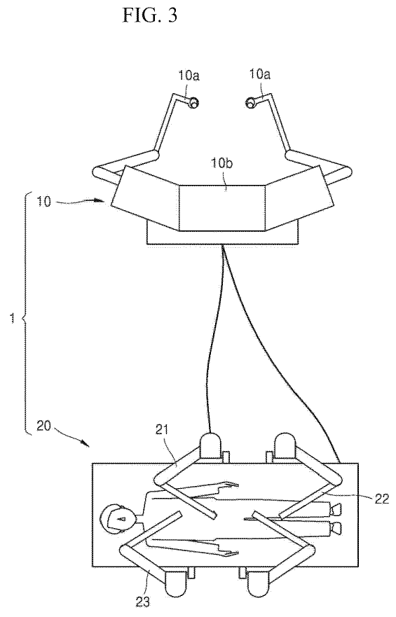

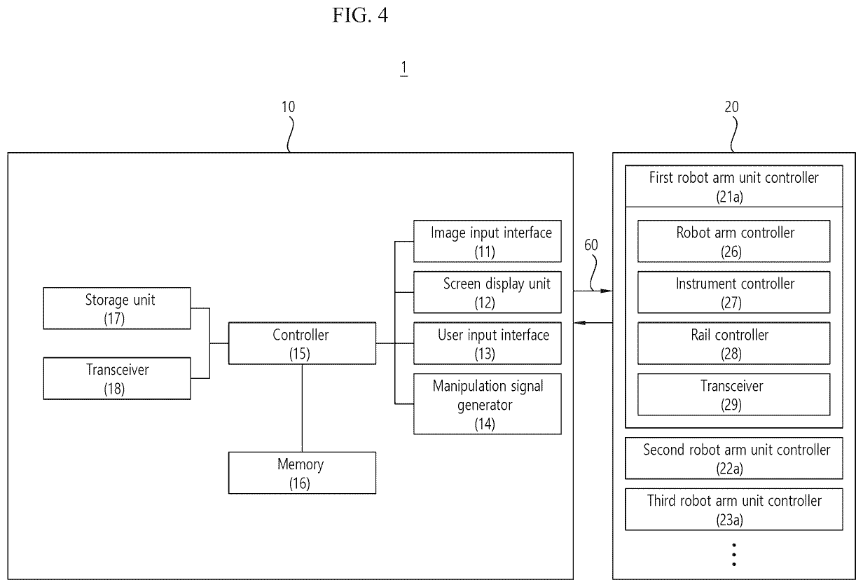

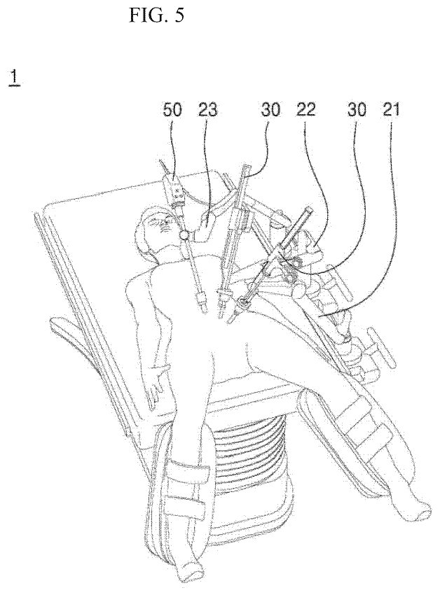

FIG. 3 is a diagram illustrating a surgical robot system according to an embodiment. FIG. 4 is a block diagram illustrating the internal configuration of the surgical robot system of FIG. 3. FIG. 5 is a perspective view of a slave robot of the surgical robot system of FIG. 3 and a surgical instrument mounted thereon.

Referring to FIGS. 3 to 5, a surgical robot system 1 includes a master robot 10, a slave robot 20, a surgical instrument 30 and a laparoscope camera 50.

The master robot 10 includes manipulation members 10a and a display member 10b, and the slave robot 20 includes one or more robot arm units 21, 22, and 23.

As a non-limiting example, the master robot 10 may include the manipulation members 10a so that a surgical operator may grip and manipulate the same respectively with both hands. The manipulation members 10a may be implemented as two or more handles as illustrated in FIG. 3, and manipulation signals according to the handle manipulation of the surgical operator are transmitted to the slave robot 20 through a wired or wireless communication network so that the robot arm units 21, 22, and 23 are controlled. In other words, surgical operations such as positioning, rotation, and cutting work of the robot arm units 21, 22, and 23 may be performed by the handle manipulation of the surgical operator. Herein, the manipulation signal may be, for example, manipulation information generated by a processor, but is not limited thereto.

For example, the surgical operator may manipulate the robot arm units 21, 22, and 23 using manipulation levers in the form of a handle. The manipulation lever as described above may have various mechanical configurations according to the manipulation method thereof, and may be provided in various configurations for operating the robot arm units 21, 22, and 23 of the slave robot 20 and/or other surgical equipment, such as a master handle manipulating the operation of each of the robot arm units 21, 22, and 23 and various input tools added to the master robot 10 for manipulating the functions of the entire system such as joystick, keypad, trackball, foot pedal, and touch screen. Herein, the manipulation member 10a is not limited to the shape of a handle and may be applied without any limitation as long as the manipulation member 10a may control operations of the robot arm units 21, 22, and 23 through a network such as a wired or wireless communication network.

According to an embodiment of the present disclosure, manipulation information may be generated based on the manipulation lever or manipulation member 10a described above. For example, according to an embodiment of the present disclosure, manipulation information may be generated based on the operation of a user manipulating the manipulation lever or manipulation member 10a. However, examples of generating manipulation information are not limited to the above description.

Alternatively, a voice input or a motion input may also be applied as user input. In other words, a user may wear, on the head thereof, glasses or a head mount display (HMD), to which a sensor is attached, and a laparoscope camera 50 may move according to a direction of the gaze. Alternatively, when the user issues a command with voice, such as “left”, “right”, “first arm”, “second arm”, and the like, the voice command may be recognized and the motion may be performed. For example, an embodiment of the present disclosure may generate manipulation information based on the voice of the user.

An image captured through the laparoscope camera 50 is displayed as a screen image on the display member 10b of the master robot 10. For example, the image captured via the laparoscope camera 50 may include a surgical site of a patient, a surgical instrument being inserted into the surgical side of a patient, a motion of the surgical instrument, and the like. For example, the display member 10b may display a video image corresponding to the motion of the surgical instrument being inserted into the surgical site of the patient. In addition, a predetermined fictive manipulation plate may be displayed independently or displayed together with the image captured by the laparoscope camera 50 on the display member 10b. The arrangement, configuration, and the like of such a fictive manipulation plate will not be described in detail.

The display member 10b may include one or more monitors, each of which may individually display information necessary for surgery. The quantity of monitors may be variously decided depending on the type or kind of information that needs to be displayed.

One or more slave robots 20 may be provided to operate a patient. As a non-limiting example, the surgical robot system 1 may include a slave robot 20 (which may be referred to as the “first robot”) coupled with a surgical instrument 30 (which may be referred to as the “first robot”) and a slave robot 20 (which may be referred to as the “second robot”) coupled with a laparoscope camera 50 (which may be referred to as the “second robot”), respectively. In other words, the laparoscope camera 50 for allowing a surgical site or a surgical instrument to be displayed as a screen image through the display member 10b may be implemented as a separate slave robot 20 independent of the slave robot 20 to which the surgical instrument 30 is coupled. It should also be understood that, as described above, the embodiments of the present disclosure may be used universally for surgeries in which various surgical endoscopes other than laparoscopes (for example, thoracoscopic, arthroscopic, rhinoscopic, and the like) are used.

In one example, two of the robot arm units 21, 22, and 23 may have the surgical instrument 30 attached thereto, and one of the robot arm units 21, 22, and 23 may have the laparoscope camera 50 attached thereto. In addition, a surgical operator may select the slave robot 20 (or the robot arm unit 21, 22, or 23) to be controlled via the master robot 10. As described above, by directly controlling a total of three or more surgical instruments through the master robot 10, the surgical operator may accurately and freely control various instruments according to the intention of the surgical operator without a surgical assistant.

As another example, the slave robot 20 may include one or more robot arm units 21, 22, and 23. Although FIGS. 3 to 5 exemplarily show one robot arm unit 21, 22, 23 coupled to one slave robot 20, it is noted that the technical spirit of the present disclosure is not limited to this. For example, two robot arm units may be coupled to one slave robot 20, with a surgical instrument 30 attached to one of the robot arm units and a laparoscope camera 50 attached to the other robot arm unit. However, even when a plurality of robot arm unit are coupled to a single slave robot 20, each of the robot arm units 21, 22, and 23 may be provided in the form of a module that may operate independently of each other, and in this connection, an algorithm for preventing a collision between the robot arm units 21, 22, and 23 may be applied to the surgical robot system 1.

The slave robot 20 may include one or more robot arm units 21, 22, and 23. Herein, each of the robot arm units 21, 22, and 23 may be provided in the form of a module that may operate independently of each other, and in this connection, an algorithm for preventing a collision between the robot arm units 21, 22, and 23 may be applied to the surgical robot system 1.

In general, a robot arm refers to an apparatus having a function similar to that of the arm and/or the wrist of a human being and having a wrist portion to which a predetermined tool may be attached. In an embodiment of the present disclosure, the robot arm units 21, 22, and 23 may each be defined as a concept encompassing all of the components such as an upper arm, a lower arm, a wrist, and an elbow, a surgical instrument (or a laparoscope camera) coupled to the wrist portion, and the like. Alternatively, the robot arm unit may also be defined as a concept that includes only components for driving the surgical instrument (or a laparoscope camera), excluding the surgical instrument (or a laparoscope camera) coupled to the wrist portion.

The robot arm units 21, 22, and 23 of the slave robot 20 described above may be implemented to be driven with multiple degrees of freedom. The robot arm units 21, 22, and 23 may include, for example, a surgical instrument (or a laparoscope) inserted into a surgical site of a patient, a yaw driving unit for rotating the surgical instrument in a yaw direction according to a surgical position, a pitch driving unit for rotating the surgical instrument in a pitch direction perpendicular to a rotational driving of the yaw driving unit, a transfer driving unit for moving the surgical instrument in a length direction, a rotation driving unit for rotating the surgical instrument, and a surgical instrument driving unit for incising or cutting the surgical lesion by driving an end effector at an end of the surgical instrument. However, the configuration of the robot arm units 21, 22, and 23 is not limited thereto, and it should be understood that this example does not limit the scope of the present disclosure. Herein, a detailed description of the actual control process, such as rotation and movement of the robot arm units 21, 22, and 23 in a corresponding direction by the surgical operator manipulating the manipulating member 10a, will be omitted.

The master robot 10 may perform various activities such as at least one of generating manipulation information based on an amount of change in the reference posture of the user input interface for controlling the surgical instrument, deciding the target posture of the surgical instrument corresponding to the manipulation information, deciding the target state information for the driving element, or driving the driving element according to the target state information.

For example, the master robot 10 transmits at least one piece of the manipulation information or the target state information of the driving element determined based thereon to the slave robot 20 through a wired or wireless communication network to control the robot arm units 21, 22, and 23. In other words, surgical operations such as positioning, rotation, and cutting work of the robot arm units 21, 22, and 23 may be performed by the handle manipulation of a surgical operator. In other words, when the manipulation information is decided by the master robot 10, the decided manipulation information may be transmitted to the slave robot 20 through a wired or wireless communication network, and the slave robot 20 may decide the target state information based on the manipulation information. According to another aspect, the master robot 10 may decide manipulation information, decide target state information corresponding thereto, and transmit the decided target state information to the slave robot 20.

Referring to FIG. 4, in an embodiment of the present disclosure, the master robot 10 may include an image input interface 11, a screen display unit 12, a user input interface 13, a manipulation signal generator 14, a controller 15, a memory 16, a storage unit 17, and a transceiver 18.

At least some of the configurations of the master robot 10 may be included in the user terminal of FIG. 2A. For example, the manipulation signal generator 14 and the controller 15 may be included in the processor 2011, the memory 16 and the storage unit 17 may be included in the memory 2012, and the transceiver 18 may be included in the communication module 2014, but the example of the master robot 10 is not limited to the above.

The image input interface 11 may receive an image captured by a camera provided in the laparoscope camera 50 of the slave robot 20 through a wired or wireless communication network. For example, the images captured through the laparoscope camera 50 may include images of a surgical site of a patient, surgical instruments being inserted into the surgical site of the patient, the motion of the surgical instruments, and the like. Further, such images may include an image representing the operation of the surgical instrument driven according to target state information.

The screen display unit 12 outputs a screen image corresponding to the image received through the image input interface 11 as visual information. In addition, the screen display unit 12 may further output information corresponding to biometric information of a subject to be treated, when the biometric information is input. In addition, the screen display unit 12 may further output image data (for example, an X-ray image, a CT image, an MRI image, or the like) associated with a patient for a surgical site. Herein, the screen display unit 12 may be implemented in the form of a display member (see 10b of FIG. 3), and an image processing process for allowing the received image to be output as a screen image through the screen display unit 12 may be performed by the controller 15. Herein, the image may include an image representing the operation of the surgical instrument driven according to target state information.

In the embodiment illustrated in FIG. 4, the image input interface and the screen display unit are illustrated as being included in the master robot 10, but an embodiment of the present disclosure is not limited thereto. The display member may be provided as a separate member spaced apart from the master robot 10. Alternatively, the display member may be provided as one component of the master robot 10. In addition, in another embodiment, a plurality of display members may be provided, one of which may be disposed adjacent to the master robot 10, and others thereof may be disposed at some distance from the master robot 10.

Herein, the screen display unit 12 (in other words, the display member 10b of FIG. 3) may be provided as a three-dimensional display apparatus. In detail, the three-dimensional display apparatus refers to an image display apparatus in which depth information is added to a two-dimensional image by applying a stereoscopic technique, and this depth information is used to enable an observer to feel a three-dimensional living feeling and a sense of reality. The surgical robot system 1 according to an embodiment of the present disclosure may provide a more realistic fictive environment to a user by including a three-dimensional display apparatus as the screen display unit 12.

The user input interface 13 is a member for allowing a surgical operator to manipulate the positions and functions of the robot arm units 21, 22, and 23 of the slave robot 20. The user input interface 13 may be formed in the form of a handle-shaped manipulation member (see 10a of FIG. 3) as illustrated in FIG. 3, but the shape thereof is not limited thereto and may be implemented by being modified in various shapes to achieve the same purpose. In addition, for example, some of the user input interface 13 may be formed in the shape of a handle, and the others thereof may be formed in a different shape, such as a clutch button. In addition, a finger insertion tube or insertion ring may be further formed so as to allow the fingers of a surgical operator to be inserted therethrough and fixed to facilitate manipulation of a surgical instrument.

According to an embodiment of the present disclosure, manipulation information may be generated based on the operation of a surgical operator on the user input interface 13. For example, according to an embodiment of the present disclosure, manipulation information can be generated based on the operation of the surgical operator manipulating the user input interface 13. However, examples of generating manipulation information are not limited to the above.

The manipulation signal generator 14 generates a corresponding manipulation signal when a surgical operator manipulates the user input interface 13 to move the position of the robot arm units 21, 22, and 23 or manipulate the surgical operation. As an example, the manipulation signal generator 14 may generate corresponding manipulation information when the surgical operator manipulates the user input interface 13 to move the position of the robot arm units 21, 22, and 23 or manipulate the surgical operation.

For example, the manipulation signal generator 14 transmits the generated manipulation signal to the controller 15 or to the slave robot 20 through the transceiver 18. The manipulation signal may be transmitted and received through a wired or wireless communication network. Based on the transmitted manipulation signal, the controller 15 may control the slave robot 20, the surgical instrument 30, or the laparoscope camera 50 to operate. Alternatively, based on the transmitted manipulation signal, a robot arm controller 26 included in the slave robot 20 may control the robot arm units 21, 22, and 23 to operate. Alternatively, based on the transmitted manipulation signal, an instrument controller 27 included in the slave robot 20 may control the surgical instrument 30 or laparoscope camera 50 to operate. However, the method by which the operation of the slave robot 20, the surgical instrument 30, or the laparoscope camera 50 is controlled based on the manipulation signal is not limited to the aforementioned method.

The instrument controller 27 receives the manipulation signal generated by the manipulation signal generator 14 of the master robot 10 and controls the surgical instrument 30 to operate according to the manipulation signal.

The controller 15 is a kind of central processing device, and controls the operation of each component so that the aforementioned functions may be performed. In an example, the controller 15 may perform a function of transforming an image input through the image input interface 11 into a screen image to be displayed through the screen display unit 12. As another example, the controller 15 may generate the target posture of the robot arm units 21, 22, and 23 based on manipulation information. In addition, the controller 15 may decide target state information of the at least one driving element based on the target posture. In addition, the controller 15 may drive the robot arm units 21, 22, and 23 based on the decided target state information.

According to the above description, it has been described that the controller 15 calculates the target posture based on the manipulation information and target state information, which may be performed by other controllers according to an embodiment of the present disclosure (for example, by the robot arm controller 26, or the instrument controller 27), without being limited thereto.

The memory 16 may perform a function of temporarily or permanently storing data processed by the controller 15. Herein, the memory 16 may include a magnetic storage medium or a flash storage medium, but the scope of the present disclosure is not limited thereto.

The storage unit 17 may store data received from the slave robot 20. In addition, the storage unit 17 may store various pieces of input data (for example, patient data, device data, surgery data, and the like).

The transceiver 18 interworks with a communication network 60 to provide a communication interface necessary for transmitting and receiving image data transmitted from the slave robot 20 and control data transmitted from the master robot 10. The image data transmitted from the slave robot 20 may include an image representing the operation of the surgical instrument driven according to target state information. The control data transmitted from the master robot 10 may include at least one piece of manipulation information on an amount of change in the user input interface or target state information on an operation of the slave robot 20.

The slave robot 20 includes a plurality of robot arm unit controllers 21a, 22a, and 23a. In addition, the robot arm unit controller 21a includes a robot arm controller 26, an instrument controller 27, and a transceiver 29. Further, the robot arm unit controllers 21a may further include a rail controller 28.

Referring to FIGS. 4 and 5, the rail controller 28 may control the path of movement of the surgical instrument 30 on the robot arm units 21, 22, 23 to enable movement along a preset path, specifically along the longitudinal direction of the connection part 310 described later herein.

The robot arm controller 26 may receive a manipulation signal generated by the manipulation signal generator 14 of the master robot 10, and may serve to control the robot arm units 21, 22, and 23 to operate according to the manipulation signal. For example, the robot arm controller 26 may receive manipulation information or target state information calculated from the master robot 10, and may serve to control the robot arm units 21, 22, and 23 to operate accordingly.

The instrument controller 27 may receive a manipulation signal generated by the manipulation signal generator 14 of the master robot 10, and may serve to control the surgical instrument 30 to operate according to the manipulation signal. For example, the instrument controller 26 may receive manipulation information or target state information calculated from the master robot 10, and may serve to control the surgical instrument 30to operate accordingly.

The transceiver 29 interworks with the communication network 60 to provide a communication interface necessary for transmitting and receiving image data transmitted from the slave robot 20 and control data transmitted from the master robot 10. The image data transmitted from the slave robot 20 may include an image representing the operation of the surgical instrument driven according to target state information. The control data transmitted from the master robot 10 may include at least one piece of manipulation information on an operation of the slave robot 20 or target state information.

The communication network 60 serves to connect the master robot 10 and the slave robot 20. In other words, the communication network 60 refers to a communication network for providing an access path so that data may be transmitted and received between the master robot 10 and the slave robot 20 after the master robot 10 and the slave robot 20 are connected. The communication network 60 may be, for example, a wired network such as local area networks (LANs), wired area networks (WANs), metropolitan area networks (MANs), and integrated service digital networks (ISDNs), or a wireless network such as wireless LANs, code division multiple access (CDMA), Bluetooth, and satellite communication, but the scope of an embodiment of the present disclosure is not limited thereto.

### Modular Slave Robot

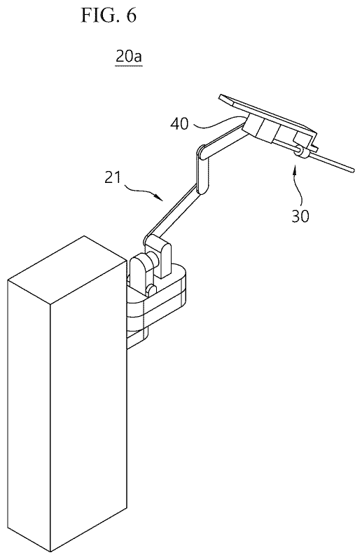

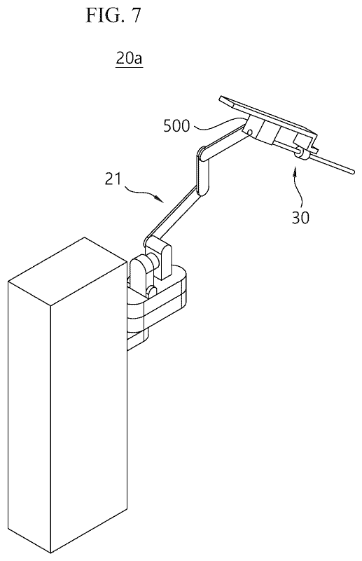

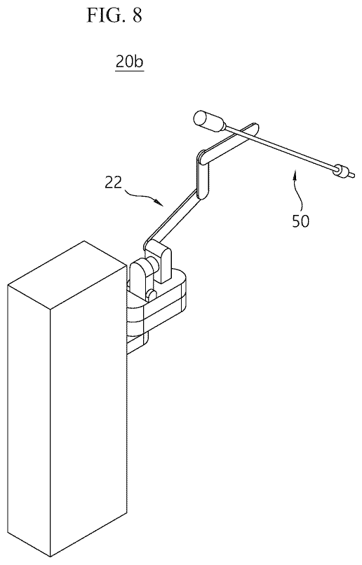

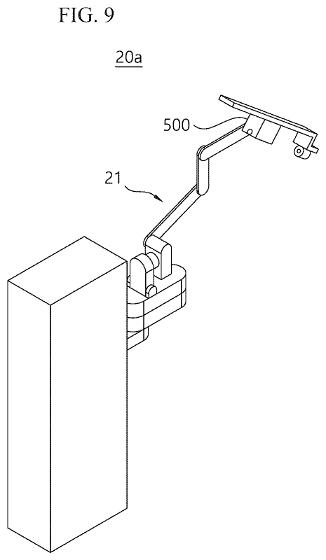

FIG. 6 is a perspective view of a modular slave robot and a surgical instrument mounted thereon according to an aspect of the surgical robot system of FIG. 3. FIG. 7 is a diagram illustrating a state in which the instrument case is removed from FIG. 6. FIG. 8 is a perspective view of a modular slave robot and a laparoscopic surgical camera mounted thereon according to another aspect of the surgical robot system of FIG. 3. FIG. 9 is a diagram illustrating a state in which the surgical instrument is removed from the slave robot of FIG. 6.

The surgical instrument 30 or the laparoscope camera 50, which will be described below, may be connected to and installed in the robot arm unit 21, 22, or 23. Referring to FIG. 6, an instrument case 40 may cover the surgical instrument 30, and may be connected to the robot arm unit 21. The instrument case 40 may cover one side of the surgical instrument 30 exposed to the outside, so as to prevent external foreign substances from reaching the surgical instrument 30, and protect the surgical instrument 30 from being damaged due to external shock.

Referring to FIG. 7, the surgical instrument 30 may be connected to and installed in the robot arm unit 21 of a modular slave robot 20a according to an embodiment. In an embodiment of the present disclosure, the modular slave robot 20a in which the surgical instrument 30 is installed in the robot arm unit 21 may be referred to as a “surgical robot.” Referring to FIG. 8, the laparoscope camera 50 may be connected to and installed in the robot arm unit 22 of the modular slave robot 20b according to an embodiment. In an embodiment of the present disclosure, the modular slave robot 20b in which the laparoscope camera 50 is installed in the robot arm unit 22 may be referred to as a “camera robot.”

Referring to FIGS. 6 to 9, only one robot arm unit 21, 22 among the robot arm units 21, 22, 23 is exemplarily illustrated in a form in which one slave robot 20a or 20b is coupled with the surgical instrument 30 or the laparoscope camera 50, but the technical idea of the present disclosure is not limited thereto. As described above, two of the robot arm units 21, 22, 23 may be attached to the surgical instrument 30, one may be attached to the laparoscope camera 50, and two or more robot arm units may be provided for one slave robot.

Referring to FIGS. 6 to 9, a motor pack 500 is connectable to the surgical instrument 30, and may be coupled to the surgical robot 20a, specifically, the robot arm unit 21, and fixed in position.

The instrument case 40 is connected to one side of the surgical instrument 30, and the motor pack 500 is connected and coupled to the other side opposite thereto. The motor pack 500 receives power source from the outside to generate power, and may transmit the power generated from the motor pack 500 to the surgical instrument 30, thereby allowing the surgical instrument 30 to perform pitch motion, yaw motion, actuation motion, and roll motion.

### Active/passive Arm Unit

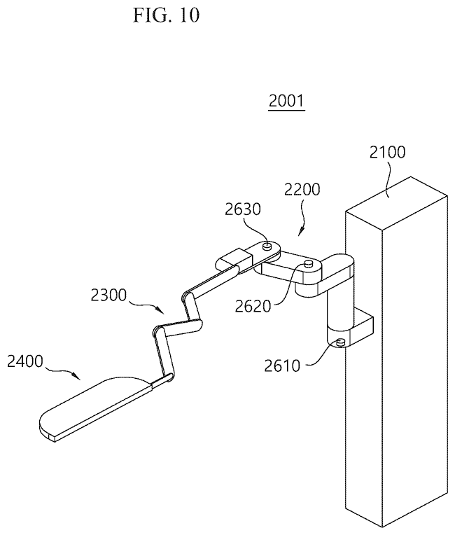

FIG. 10 is a perspective view of another example of a modular slave robot and a surgical instrument mounted thereon of a surgical robot system according to an embodiment.

Referring to FIG. 10, a surgical robot 2001 according to an embodiment may include a body 2100, an active arm unit 2300, and a surgical instrument 2400. In addition, the surgical robot 2001 according to another embodiment may further include a passive arm unit 2200 and one or more angle measuring sensors 2610, 2620, 2630.

The body 2100 may refer to a main body connected to the robot arm unit. For example, the robot arm unit and the body 2100 may configure one independent slave robot 20. In addition, the body 2100 may include a moving member (not shown) that allows the surgical robot 2001 to be disposed at a desired position in an operating room. For example, the body 2100 may be provided with wheels so as to move freely. The body 2100 may further include a fixing member (not shown) that allows the surgical robot 2001 to be fixed to the operating room and prevented from moving. For example, after the disposition of the surgical robot 2001 is completed and an surgical operator begins surgery, the fixing member may fix the body 2100 to a predetermined position in the operating room so that the surgical robot 2001 may not move for the sake of the stability of the surgery.

The robot arm unit included in the surgical robot 2001 may include at least one of a passive arm unit 2200 or an active arm unit 2300. For example, the surgical robot 2001 may be configured of the body 2100 and the active arm unit 2300, or may be configured of the body 2100, the passive arm unit 2200, and the active arm unit 2300. For example, when the robot arm unit of the surgical robot 2001 is configured only of the active arm unit 2300, the active arm unit 2300 may be directly connected to the body 2100. As another example, when the robot arm unit of the surgical robot 2001 is configured of the passive arm unit 2200 and the active arm unit 2300, the body 2100 may be directly connected to the passive arm unit 2200, and the passive arm unit 2200 may be connected at one end to the body 2100 and at the other end to the active arm unit 2300.

The passive arm unit 2200 may be defined as a robot arm whose position, direction, angle, or the like are manipulated by external force. For example, an surgical operator or a surgical assistant assisting the surgical operator may manipulate the movement of the passive arm unit 2200 by applying physical force. In addition, the position, direction, angle, or the like of the passive arm unit 2200 may be maintained when there is no external force manipulating the movement. In other words, when the aforementioned surgical operator or surgical assistant manipulates the position, direction, angle, or the like before the surgery begins, the position, direction, angle, or the like of the passive arm unit 2200 may be maintained without change during the surgery. From this perspective, the body 2100 may be included in the passive arm unit 2200 in that the position to which the surgical operator or surgical assistant moves the body 2100 before the surgery begins may be maintained without change during the surgery.

The passive arm unit 2200 may include an angle measurement sensor 2610, 2620, 2630. Herein, the angle measurement sensor 2610, 2620, 2630 may refer to a sensor that monitors the movement of the passive arm unit 2200. For example, the angle measurement sensor 2610, 2620, 2630 may measure or calculate the position, direction, angle, etc. of the passive arm unit 2200. For example, the angle measurement sensor 2610, 2620, 2630 may be implemented as a sensor capable of measuring the change amount in position, speed, and direction of an object, such as a rotary encoder, a linear encoder, or a potentiometer.

In addition, the angle measurement sensor 2610, 2620, 2630 may be installed so as to be positioned between any two passive arm units. For example, the number of angle measurement sensors included in the surgical robot 2001 may be one less than the number of the passive arm units 2200. Referring to FIG. 10, the passive arm unit 2200 connecting the body 2100 and the active arm unit 2300 may include a total of four robot arms, and the surgical robot 2001 according to an embodiment may include a total of three angle measurement sensors.

The active arm unit 2300 may be defined as a robot arm in which the position, direction, angle, or the like of the robot arm are automatically manipulated through an internal control algorithm. For example, when a surgical operator manipulates the user input interface 13 to manipulate the active arm unit 2300, the manipulation signal generator 14 may generate a manipulation signal corresponding to the motion of the surgical operator manipulating the user input interface 13 and transmit the same to the robot arm controller of the active arm unit 2300.

Thereafter, the robot arm controller of the active arm unit 2300 may control the active arm unit 2300 to move in position, rotate, or the like according to the control algorithm based on the received control signal. In other words, the position, direction, angle, or the like of the active arm unit 2300 may be manipulated when there is manipulation by the surgical operator, regardless of before or after the start of surgery. Since the active arm unit 2300 is manipulated through a control algorithm rather than external force, an external energy supply through a motor or actuator is needed. Accordingly, the active arm unit 2300 may include one or more motors or actuators.

The surgical instrument 2400 included in the surgical robot 2001 may be connected to at least one of the passive arm unit 2200 and the active arm unit 2300. FIG. 10 illustrates the surgical robot 2001 to which a surgical instrument 2400 is coupled, but is not limited thereto. In other words, the contents described with reference to FIG. 10 may be equally applied to a camera robot to which a laparoscopic surgical camera (not shown) is coupled.

### Surgical Instrument

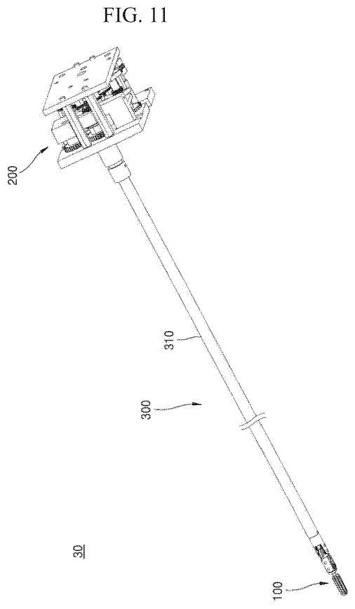

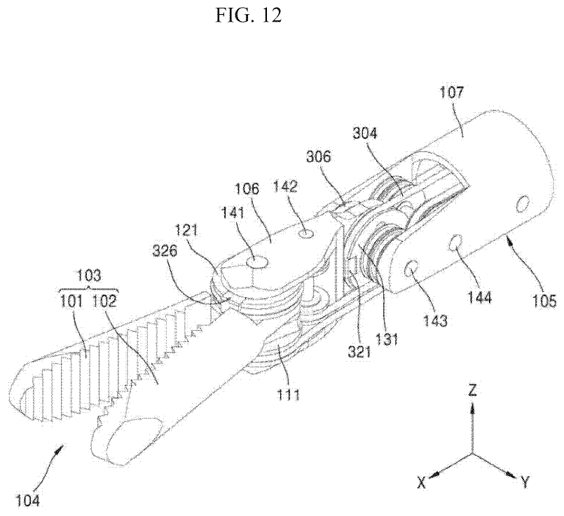

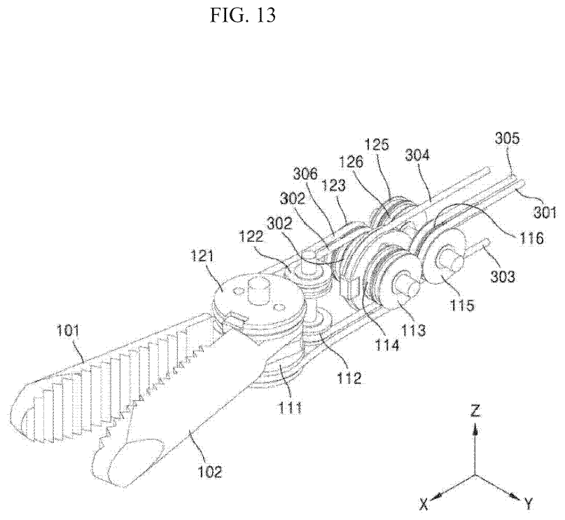

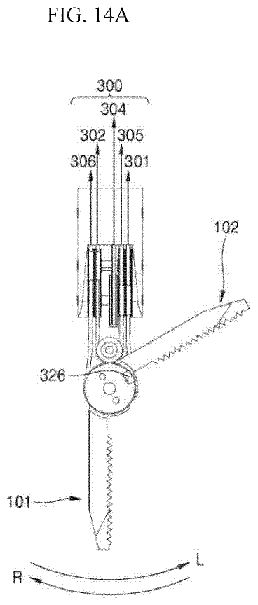

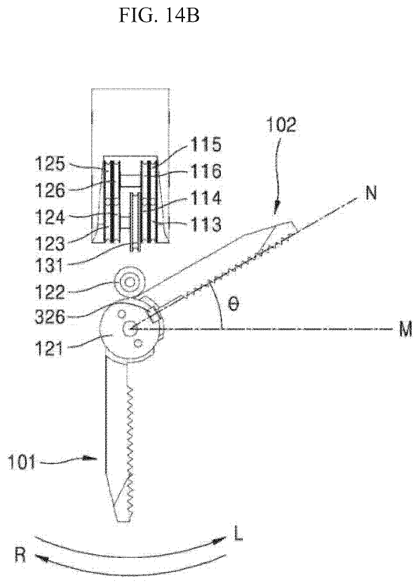

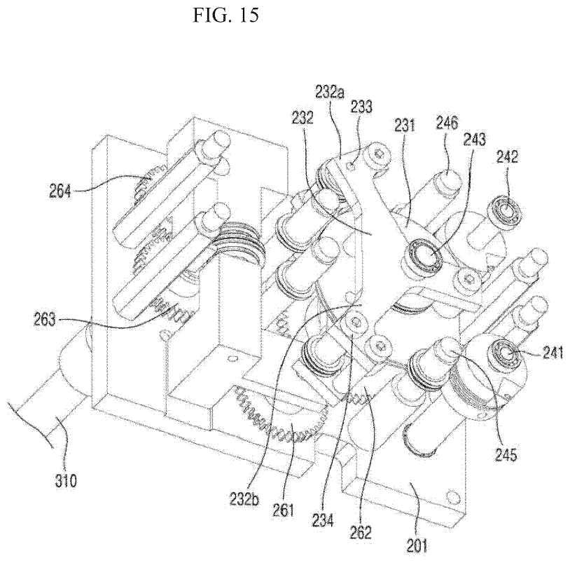

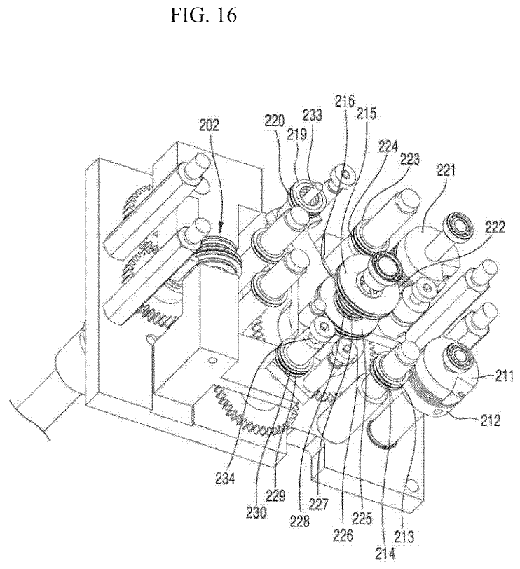

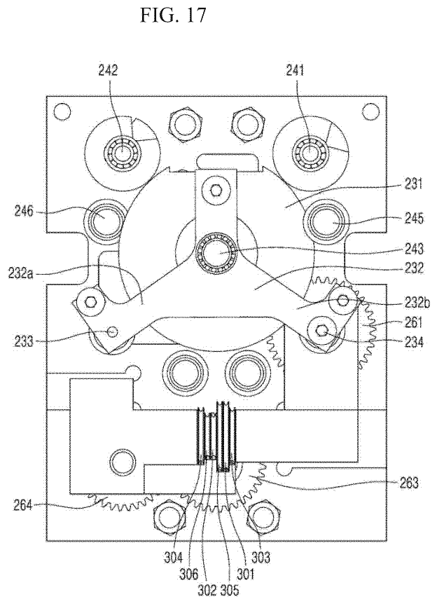

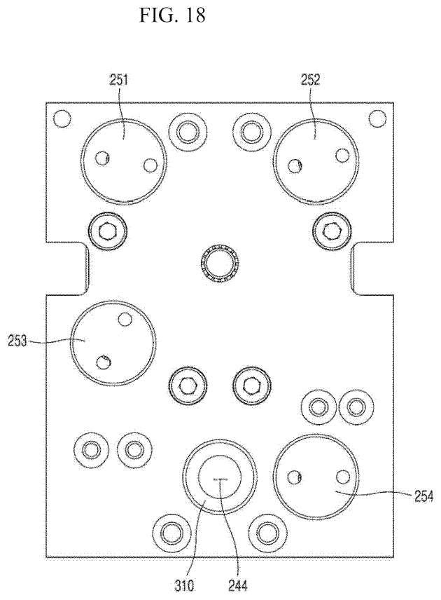

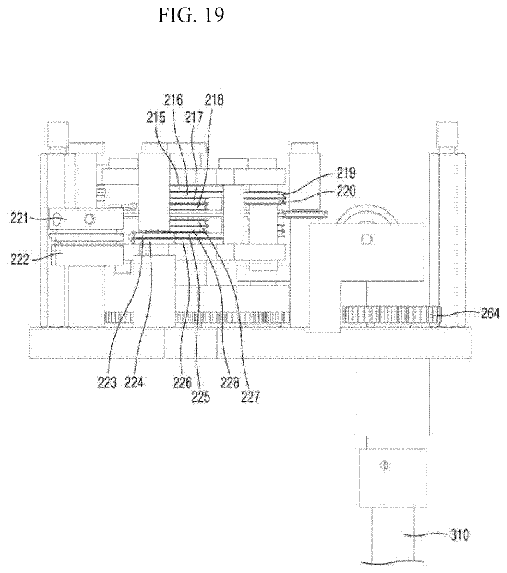

FIG. 11 is a perspective view of a surgical instrument according to an embodiment of the present disclosure, FIGS. 12 and 13 are perspective views of an end tool of the surgical instrument of FIG. 11, and FIGS. 14A to 14B is a plan view of the end tool of the surgical instrument of FIG. 11. FIGS. 15 and 16 are perspective views of a driving part of the surgical instrument of FIG. 11, FIG. 17 is a plan view of the driving part of the surgical instrument of FIG. 11, FIG. 18 is a rear view of the driving part of the surgical instrument of FIG. 11, and FIG. 19 is a side view of the driving part of the surgical instrument of FIG. 11.

Referring first to FIG. 11, the surgical instrument 30 according to an embodiment of the present disclosure may include an end tool 100, a driving part 200, and a power transmission part 300, and the power transmission part 300 may include a connection part 310.

The connection part 310 is formed in the shape of a hollow shaft, in which one or more wires (to be described later) may be accommodated, and may have one end portion to which the driving part 200 is coupled and the other end portion to which the end tool 100 is coupled, and serve to connect the driving part 200 and the end tool 100.

The driving part 200 is formed at one end portion of the connection part 310 and provides an interface capable of being coupled to the robot arm unit (see 21 or the like in FIG. 3). Accordingly, when a user operates the master robot (see 10 in FIG. 3), a motor (not shown) of the robot arm unit (see 21 or the like in FIG. 3) is operated so that the end tool 100 of the surgical instrument 30 can perform a motion corresponding thereto, and a driving force of the motor (not shown) is transmitted to the end tool 100 through the driving part 200. In other words, it may be described that the driving part 200 itself becomes an interface that connects between the surgical instrument 30 and the slave robot 20.

For example, when the user input part 13 (see FIG. 3) is operated by a user, a motor (not shown) of the robot arm unit 21 or the like (see FIG. 3) operates so that the end tool 100 of the surgical instrument 30 can perform a motion corresponding thereto, and a driving force of the motor (not shown) may be transmitted to the end tool 100 through the driving part 200.

The end tool 100 is formed on the other end portion of the connection part 310, and performs necessary motions for surgery by being inserted into a surgical site. In an example of the above-described end tool 100, as shown in FIG. 12, a pair of jaws 101 and 102 for performing a grip motion may be used. However, the embodiment of the present disclosure is not limited thereto, and various devices for performing surgery may be used as the end tool 100. For example, a configuration such as a cantilever cautery may also be used as the end tool. The above-described end tool 100 is connected to the driving part 200 by the power transmission part 300 and receives a driving force through the power transmission part 300 to perform a motion necessary for surgery, such as a gripping motion, a cutting motion, a suturing motion, or the like.

Here, the end tool 100 of the surgical instrument 30 according to an embodiment of the present disclosure is formed to be rotatable in at least two or more directions, for example, the end tool 100 may be formed to perform a pitch motion around a rotation shaft 143 of FIG. 12 and simultaneously perform a yaw motion and an actuation motion around a rotation shaft 141 of FIG. 12.

Here, each of a pitch motion, a yaw motion, an actuation motion, and a roll motion as used in the present disclosure are defined as follows.

First, the pitch motion means a motion of the end tool 100 rotating in a vertical direction with respect to an extension direction of the connection part 310 (an X-axis direction of FIG. 11), that is, a motion rotating around the Y-axis of FIG. 11. In other words, the pitch motion means a motion of the end tool 100, which is formed to extend from the connection part 310 in the extension direction of the connection part 310 (the X-axis direction of FIG. 11), rotating vertically around the Y-axis with respect to the connection part 310.

Next, the yaw motion means a motion of the end tool 100 rotating in left and right directions, that is, a motion rotating around a Z-axis of FIG. 11, with respect to the extension direction of the connection part 310 (the X-axis direction of FIG. 11). In other words, the yaw motion means a motion of the end tool 100, which is formed to extend from the connection part 310 in the extension direction of the connection part 310 (the X-axis direction of FIG. 11), rotating horizontally around the Z-axis with respect to the connection part 310. That is, the yaw motion relates to a motion of two jaws 101 and 102, which are formed on the end tool 100, rotating around the Z-axis in the same direction.

Meanwhile, the actuation motion means a motion of the end tool 100 rotating around the same shaft of rotation as that of the yaw motion, while the two jaws 101 and 102 rotate in the opposite directions so as to be closed or opened. That is, the actuation motion means rotating motions of the two jaws 101 and 102, which are formed on the end tool 100, in the opposite directions around the Z-axis.

Defining this from another perspective, the yaw rotation may be defined as a motion in which an end tool jaw pulley (to be described later) rotates around the rotation shaft 141, which is an end tool jaw pulley rotation shaft, and the pitch rotation may be defined as a motion in which the end tool jaw pulley revolves around the rotation shaft 143, which is an end tool pitch rotation shaft.

The roll motion refers to a motion in which the surgical instrument rotates with the connection part 310 as a shaft. For example, the roll motion may be a motion in which the surgical instrument rotates in the clockwise or counterclockwise direction around the extension direction of the connection part 310 (the X-axis direction of FIG. 11).

Meanwhile, the roll motion may mean a motion in which the end tool 100 rotates around the X-axis with respect to the connection part 310. For example, the roll motion may be a motion in which the end tool rotates in the clockwise or counterclockwise direction around the extension direction of the connection part 310 (the X-axis direction of FIG. 12).

The power transmission part 300 may connect the driving part 200 and the end tool 100, transmit the driving force from the driving part 200 to the end tool 100, and include a plurality of wires, pulleys, links, sections, gears, or the like.

Hereinafter, the end tool 100, the driving part 200, the power transmission part 300, and the like of the surgical instrument 30 of FIG. 11 will be described in more detail.

Hereinafter, the power transmission part 300 of the surgical instrument 30 of FIG. 11 will be described in more detail.

Referring to FIGS. 11 to 19, the power transmission part 300 of the surgical instrument 30 according to an embodiment of the present disclosure may include a plurality of wires 301, 302,303, 304, 305, and 306.

Here, the wires 301 and 305 may be paired to serve as first jaw wires. The wires 302 and 306 may be paired to serve as second jaw wires. Here, the components encompassing the wires 301 and 305, which are first jaw wires, and the wires 302 and 306, which are second jaw wires, may be referred to as jaw wires. In addition, the wires 303 and 304 may be paired to serve as pitch wires.

Here, in the drawings, a pair of wires are illustrated as being associated with a rotational motion of a first jaw 101, and a pair of wires are illustrated as being associated with a rotational motion of a second jaw 102, but an embodiment of the present disclosure is not limited thereto. For example, a pair of wires may be associated with a yaw motion, and a pair of wires may be associated with an actuation motion.

In addition, the power transmission part 300 of the surgical instrument 30 according to an embodiment of the present disclosure may include a coupling member 321, a coupling member 326, and the like, which are coupled to respective end portions of the wires in order to couple the wires and the pulleys. Here, each of the coupling members may have various shapes as necessary, such as a ball shape, a tube shape, and the like.

Here, the coupling member 321, which is a pitch wire coupling member, is coupled to the end portions of the wires 303 and 304, which are pitch wires, at the end tool 100 side to serve as a pitch wire-end tool coupling member. Meanwhile, although not illustrated in the drawings, a pitch wire-driving part coupling member (not shown) may be coupled to the end portions of the wires 303 and 304, which are pitch wires, at the driving part 200 side.

Meanwhile, the coupling member 326, which is a second jaw wire coupling member, is coupled to the end portions of the wires 302 and 306, which are second jaw wires, at the end tool 100 side to serve as a second jaw wire-end tool coupling member. Meanwhile, although not illustrated in the drawings, a second jaw wire-driving part coupling member (not shown) may be coupled to the end portions of the wires 302 and 306, which are second jaw wires, at the driving part 200 side.

Meanwhile, although not illustrated in the drawings, a coupling member (not shown) having the same shape as the second jaw wire coupling member 326 may be coupled to the end portions of the wires 301 and 305, which are first jaw wires, at the end tool 100 side to serve as a first jaw wire-end tool coupling member. Meanwhile, although not illustrated in the drawings, a first jaw wire-driving part coupling member (not shown) may be coupled to the end portions of the wires 301 and 305, which are first jaw wires, at the driving part 200 side.

Here, each of the coupling members is classified as being included in the power transmission part 300, but the coupling members may be classified such that the coupling member at the end tool 100 side may be included in the end tool 100, and the coupling member at the driving part 200 side may be included in the driving part 200.

The coupling relationship between the wires, the fastening members, and the respective pulley will be described in detail as follows.

First, the wires 302 and 306, which are second jaw wires, may be a single wire. The second jaw wire coupling member 326, which is a second jaw wire-end tool coupling member, is inserted at an intermediate point of the second jaw wire, which is a single wire, and the second jaw wire coupling member 326 is crimped and fixed, and then, both strands of the second jaw wire centered on the second jaw wire coupling member 326 may be referred to as the wire 302 and the wire 306, respectively.

Alternatively, the wires 302 and 306, which are second jaw wires, may also be formed as separate wires, and connected to each other by the second jaw wire coupling member 326.

In addition, by coupling the second jaw wire coupling member 326 to a pulley 121, the wires 302 and 306 may be fixedly coupled to the pulley 121. This allows the pulley 121 to rotate as the wires 302 and 306 are pulled and released.

Meanwhile, the second jaw wire-driving part coupling member (not shown) may be coupled to the end portions of the wires 302 and 306, which are opposite to the end portions to which the second jaw wire coupling member 326 is coupled. That is, the second jaw wire-driving part coupling member (not shown) may be fixed to each of the wires 302 and 306 by inserting the opposite end portions of the wires 302 and 306 into the second jaw wire-driving part coupling member (not shown) and crimping the coupling member (not shown).

In addition, by coupling the second jaw wire-driving part coupling member (not shown) coupled to the wires 302 and 306 to each of the pulley 221 and the pulley 222, the wire 302 and the wire 306 may be fixedly coupled to the pulley 221 and the pulley 222, respectively. As a result, when the pulley 221 and the pulley 222 are rotated by a motor or a human force, the pulley 121 of the end tool 100 may be rotated as the wire 302 and the wire 306 are pulled and released.

Here, a driving part second jaw pulley may include two pulleys of the pulley 221 and the pulley 222, and thus the second jaw wire-driving part coupling member may also include two coupling members. Alternatively, the driving part second jaw pulley includes one pulley, the second jaw wire-driving part coupling member also includes one coupling member, and the wires 302 and 306 may be coupled to one coupling member to be coupled to one driving part second jaw pulley.

In the same manner, the wire 301 and the wire 305, which are first jaw wires, are coupled to the first jaw wire-end tool coupling member (not shown) and the first jaw wire-driving part coupling member (not shown), respectively. In addition, the first jaw wire-end tool coupling member (not shown) is coupled to a pulley 111, and the first jaw wire-driving part coupling member (not shown) is coupled to a pulley 211 and a pulley 212. As a result, when the pulleys 211 and 212 are rotated by a motor or a human force, the pulley 111 of the end tool 100 may be rotated as the wire 301 and the wire 305 are pulled and released.

In the same manner, each of one end portions of the wires 303 and 304, which are pitch wires, is coupled to the pitch wire coupling member 321, which is a pitch wire-end tool coupling member, and each of the other end portions of the wires 303 and 304 are coupled to the pitch wire-driving part coupling member (not shown). In addition, the pitch wire coupling member 321 is coupled to a pulley 131, and the pitch wire-driving part coupling member (not shown) is coupled to a pulley 231. As a result, when the pulley 231 is rotated by a motor or a human force, the pulley 131 of the end tool 100 may be rotated as the wire 303 and the wire 304 are pulled and released.

As a result, the wire 301 and the wire 305, which are both strands of the first jaw wire, are coupled to a coupling member 323, which is a first jaw wire-end tool coupling member, and the first jaw wire-driving part coupling member (not shown) so as to form as a whole a closed loop. Similarly, the second jaw wire and the pitch wire may each be formed to form a closed loop.

Hereinafter, the end tool 100 of the surgical instrument 30 of FIG. 11 will be described in more detail.

FIGS. 12 and 13 are perspective views of the end tool of the surgical instrument of FIG. 11, and FIGS. 14A to 14B is a plan view of the end tool of the surgical instrument of FIG. 11. Here, FIG. 12 illustrates a state in which an end tool hub 106 and a pitch hub 107 are coupled, and FIG. 13 illustrates a state in which the end tool hub 106 and the pitch hub 107 are removed.

Referring to FIGS. 12 to 14, the end tool 100 according to an embodiment of the present disclosure includes a pair of jaws for performing a grip motion, that is, the first jaw 101 and the second jaw 102. Here, each of the first jaw 101 and the second jaw 102, or a component encompassing the first jaw 101 and the second jaw 102 may be referred to as a jaw 103.

Further, the end tool 100 may include the pulley 111, a pulley 112, a pulley 113, a pulley 114, a pulley 115, and a pulley 116 that are related to a rotational motion of the first jaw 101. In addition, the end tool 100 may include the pulley 121, a pulley 122, a pulley 123, a pulley 124, a pulley 125, and a pulley 126 that are related to a rotational motion of the second jaw 102.

Here, in the drawings, one group of pulleys are illustrated as being associated with a rotational motion of the first jaw 101, and one group of pulleys are illustrated as being associated with a rotational motion of the second jaw 102, but an embodiment of the present disclosure is not limited thereto. For example, one group of pulleys in the end tool may be associated with a yaw motion, and one group of pulleys in the end tool may be associated with an actuation motion. Here, the pulleys included in the end tool 100, including the pulleys described above, may be collectively referred to as end tool pulleys.

Meanwhile, the pulleys facing each other are illustrated in the drawings as being formed parallel to each other, but an embodiment of the present disclosure is not limited thereto, and each of the pulleys may be variously formed with a position and a size suitable for the configuration of the end tool.

Further, the end tool 100 according to an embodiment of the present disclosure may include the end tool hub 106 and the pitch hub 107.

The rotation shaft 141 and a rotation shaft 142, which will be described later, may be inserted through the end tool hub 106, and the end tool hub 106 may internally accommodate at least some of the first jaw 101 and the second jaw 102, which are axially coupled to the rotation shaft 141. In addition, the end tool hub 106 may internally accommodate at least some of the pulley 112 and the pulley 122 that are axially coupled to the rotation shaft 142.

In addition, the pulley 131 serving as an end tool pitch pulley may be formed at one end portion of the end tool hub 106. As shown in FIG. 12, the pulley 131 may be formed as a separate member from the end tool hub 106 and coupled to the end tool hub 106. Alternatively, although not illustrated in the drawings, the pulley 131 may be integrally formed with the end tool hub 106 as one body. That is, one end portion of the end tool hub 106 is formed in a disk shape or a semi-circular shape such as a pulley, and a groove around which a wire can be wound may be formed on an outer circumferential surface thereof. The wires 303 and 304 described above are coupled to the pulley 131 serving as an end tool pitch pulley, and a pitch motion may be performed as the pulley 131 is rotated around the rotation shaft 143.

The rotation shaft 143 and a rotation shaft 144, which will be described later, may be inserted through the pitch hub 107, and the pitch hub 107 may be axially coupled to the end tool hub 106 and the pulley 131 by the rotation shaft 143. Thus, the end tool hub 106 and the pulley 131 (coupled thereto) may be formed to be rotatable around the rotation shaft 143 with respect to the pitch hub 107.

Further, the pitch hub 107 may internally accommodate at least some of the pulley 113, the pulley 114, the pulley 123, and the pulley 124 that are axially coupled to the rotation shaft 143. In addition, the pitch hub 107 may internally accommodate at least some of the pulley 115, the pulley 116, the pulley 125, and the pulley 126 that are axially coupled to the rotation shaft 144.

Further, the end tool 100 according to an embodiment of the present disclosure may include the rotation shaft 141, the rotation shaft 142, the rotation shaft 143, and the rotation shaft 144. As described above, the rotation shaft 141 and the rotation shaft 142 may be inserted through the end tool hub 106, and the rotation shaft 143 and the rotation shaft 144 may be inserted through the pitch hub 107.

The rotation shaft 141, the rotation shaft 142, the rotation shaft 143, and the rotation shaft 144 may be arranged sequentially from a distal end 104 of the end tool 100 toward a proximal end 105 thereof. Accordingly, starting from the distal end 104, the rotation shaft 141 may be referred to as a first pin, the rotation shaft 142 may be referred to as a second pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 142 may function as an end tool jaw auxiliary pulley rotation shaft, the rotation shaft 143 may function as an end tool pitch rotation shaft, and the rotation shaft 144 may function as an end tool pitch auxiliary rotation shaft of the end tool 100.

Each of the rotation shafts 141, 142, 143, and 144 may be fitted into one or more pulleys, which will be described in detail below.

The pulley 111 functions as an end tool first jaw pulley, and the pulley 121 functions as an end tool second jaw pulley, and these two components may be collectively referred to as end tool jaw pulleys.

The pulley 111 and the pulley 121, which are end tool jaw pulleys, are formed to face each other, and are formed to be rotatable independently of each other around the rotation shaft 141, which is an end tool jaw pulley rotation shaft. Here, in the drawings, it is illustrated that the pulley 111 and the pulley 121 are formed to rotate around one rotation shaft 141, but it is of course possible that each end tool jaw pulley may be formed to be rotatable around a separate shaft. Here, the first jaw 101 may be fixedly coupled to the pulley 111 and rotated together with the pulley 111, and the second jaw 102 may be fixedly coupled to the pulley 121 and rotated together with the pulley 121. Yaw and actuation motions of the end tool 100 are performed according to the rotation of the pulley 111 and the pulley 121. That is, when the pulley 111 and the pulley 121 are rotated in the same direction around the rotation shaft 141, the yaw motion is performed, and when the pulley 111 and the pulley 121 are rotated in opposite directions around the rotation shaft 141, the actuation motion is performed.

Here, the first jaw 101 and the pulley 111 may be formed as separate members and coupled to each other, or the first jaw 101 and the pulley 111 may be integrally formed as one body. Similarly, the second jaw 102 and the pulley 121 may be formed as separate members and coupled to each other, or the second jaw 102 and the pulley 121 may be integrally formed as one body.

The pulley 112 functions as an end tool first jaw auxiliary pulley, and the pulley 122 functions as an end tool second jaw auxiliary pulley, and these two components may be collectively referred to as end tool jaw auxiliary pulleys.

Specifically, the pulley 112 and the pulley 122, which are end tool jaw auxiliary pulleys, may be additionally provided on one side of the pulley 111 and one side of the pulley 121, respectively. In other words, the pulley 112, which is an auxiliary pulley, may be disposed between the pulley 111 and the pulley 113/pulley 114. In addition, the pulley 122, which is an auxiliary pulley, may be disposed between the pulley 121 and the pulley 123/pulley 124. The pulley 112 and the pulley 122 may be formed to be rotatable independently of each other around the rotation shaft 142. Here, in the drawings, it is illustrated that the pulley 112 and the pulley 122 are formed to rotate around one rotation shaft 142, but it is of course possible that each of the pulley 112 and the pulley 122 may be formed to be rotatable around a separate shaft. Such auxiliary pulleys will be described in more detail later.

The pulley 113 and the pulley 114 function as end tool first jaw pitch main pulleys, and the pulley 123 and the pulley 124 function as end tool second jaw pitch main pulleys, and these two components may be collectively referred to as end tool jaw pitch main pulleys.

The pulley 115 and the pulley 116 function as end tool first jaw pitch sub-pulleys, and the pulley 125 and the pulley 126 function as end tool second jaw pitch sub-pulleys, and these two components may be collectively referred to as end tool jaw pitch sub-pulleys.

Hereinafter, components related to the rotation of the pulley 111 will be described.

The pulley 113 and the pulley 114 function as end tool first jaw pitch main pulleys. That is, the pulley 113 and the pulley 114 function as main rotation pulleys for a pitch motion of the first jaw 101. Here, the wire 301, which is a first jaw wire, is wound around the pulley 113, and the wire 305, which is a first jaw wire, is wound around the pulley 114.

The pulley 115 and the pulley 116 function as end tool first jaw sub-pulleys. That is, the pulley 115 and the pulley 116 function as sub rotation pulleys for a pitch motion of the first jaw 101. Here, the wire 301, which is a first jaw wire, is wound around the pulley 115, and the wire 305, which is a first jaw wire, is wound around the pulley 116.

Here, the pulley 113 and the pulley 114 are disposed on one side of the pulley 111 and the pulley 112 to face each other. Here, the pulley 113 and the pulley 114 are formed to be rotatable independently of each other around the rotation shaft 143 that is an end tool pitch rotation shaft. In addition, the pulley 115 and the pulley 116 are disposed on one side of the pulley 113 and on one side of the pulley 114, respectively, to face each other. Here, the pulley 115 and the pulley 116 are formed to be rotatable independently of each other around the rotation shaft 144 that is an end tool pitch auxiliary rotation shaft. Here, in the drawings, it is illustrated that the pulley 113, the pulley 115, the pulley 114, and the pulley 116 are all formed to be rotatable around a Y-axis direction, but an embodiment of the present disclosure is not limited thereto, and the rotation axes of the respective pulleys may be formed in various directions according to configurations thereof.

The wire 301, which is a first jaw wire, is sequentially wound to make contact with at least portions of the pulley 115, the pulley 113, and the pulley 111. In addition, the wire 305 connected to the wire 301 by the first jaw wire-end tool coupling member 323 is sequentially wound to make contact with at least portions of the pulley 111, the pulley 112, the pulley 114, and the pulley 116 in turn.

Viewed from another perspective, the wires 301 and 305, which are first jaw wires, are sequentially wound to make contact with at least portions of the pulley 115, the pulley 113, the pulley 111, the pulley 112, the pulley 114, and the pulley 116 and are formed to move along the above pulleys while rotating the above pulleys.

Accordingly, when the wire 301 is pulled in the direction of an arrow of the wire 301 of FIGS. 14A to 14B, a coupling member (not shown) to which the wire 301 is coupled and the pulley 111 coupled to the coupling member (not shown) are rotated in an arrow L direction of FIGS. 14A to 14B. In contrast, when the wire 305 is pulled in the direction of an arrow of the wire 305 of FIGS. 14A to 14B, a coupling member (not shown) to which the wire 305 is coupled and the pulley 111 coupled to the coupling member (not shown) are rotated in an arrow R direction of FIGS. 14A to 14B.

Hereinafter, the pulley 112 and the pulley 122 serving as auxiliary pulleys will be described in more detail.

The pulley 112 and the pulley 122 may serve to increase rotation angles of the first jaw 101 and the second jaw 102, respectively, by coming into contact with the wire 305, which is a first jaw wire, and the wire 302, which is a second jaw wire, and changing the arrangement paths of the wires 305 and 302 to a certain extent.

That is, when the auxiliary pulleys are not disposed, each of the first jaw and the second jaw may be rotated up to a right angle, but in an embodiment of the present disclosure, the pulley 112 and the pulley 122, which are auxiliary pulleys, are additionally provided, so that the maximum rotation angle may be increased by θ as shown in FIGS. 14A to 14B. This enables a motion of the two jaws of the end tool 100 being opened for an actuation motion while the two jaws are yaw-rotated by 90° in the L direction. This is because the second jaw 102 is rotated by the additional angle θ as shown in FIG. 12. Similarly, an actuation motion is possible even when the two jaws are yaw-rotated in the R direction. In other words, a feature of increasing the range of yaw rotation in which an actuation motion is possible may be obtained through the pulley 112 and the pulley 122.

This will be described in more detail as follows.

When the auxiliary pulleys are not disposed, since the first jaw wire is fixedly coupled to the end tool first jaw pulley, and the second jaw wire is fixedly coupled to the end tool second jaw pulley, each of the end tool first jaw pulley and the end tool second jaw pulley may be rotated up to 90°. In this case, when the actuation motion is performed while the first jaw and the second jaw are located at a 90° line, the first jaw may be opened, but the second jaw may not be rotated beyond 90°. Accordingly, when the first jaw and the second jaw perform a yaw motion over a certain angle, there was a problem that the actuation motion is not smoothly performed.

In order to address such a problem, in the surgical instrument 30 according to an embodiment of the present disclosure, the pulley 112 and the pulley 122, which are auxiliary pulleys, are additionally disposed at one side of the pulley 111 and one side of the pulley 121, respectively. As described above, as the arrangement paths of the wire 305, which is a first jaw wire, and the wire 302, which is a second jaw wire, are changed to a certain extent by disposing the pulley 112 and the pulley 122, a tangential direction of the wires 305 and 302 is changed, and accordingly, the second jaw wire coupling member 326 for coupling the wire 302 and the pulley 121 may be rotated up to a line N of FIGS. 14A to 14B. That is, the second jaw wire coupling member 326, which is a coupling part of the wire 302 and the pulley 121, is rotatable until the second jaw wire coupling member 326 is located on a common internal tangent of the pulley 121 and the pulley 122. Similarly, the first jaw wire-end tool coupling member 323, which is a coupling part of the wire 305 and the pulley 111, is rotatable until the first jaw wire-end tool coupling member 323 is located on a common internal tangent of the pulley 111 and the pulley 112, so that the range of rotation in the L direction may be increased.

In other words, by the pulley 112, the wires 301 and 305, which are two strands of the first jaw wire wound around the pulley 111, are disposed at one side with respect to a plane perpendicular to the Y-axis and passing through the X-axis. Simultaneously, by the pulley 122, the wires 302 and 306, which are two strands of the second jaw wire wound around the pulley 121, are disposed at the other side with respect to the plane perpendicular to the Y-axis and passing through the X-axis.

In other words, the pulley 113 and the pulley 114 are disposed at one side with respect to the plane perpendicular to the Y-axis and passing through the X-axis, and the pulley 123 and the pulley 124 are disposed at the other side with respect to the plane perpendicular to the Y-axis and passing through the X-axis.

In other words, the wire 305 is located on the internal tangent of the pulley 111 and the pulley 112, and the rotation angle of the pulley 111 is increased by the pulley 112. In addition, the wire 302 is located on the internal tangent of the pulley 121 and the pulley 122, and the rotation angle of the pulley 121 is increased by the pulley 122.

According the above-described embodiment of the present disclosure, as the rotation radii of the jaw 101 and the jaw 102 increase, an effect of increasing a yaw motion range in which a normal opening/closing actuation motion is performed may be obtained.

Next, components related to the rotation of the pulley 121 will be described.

The pulley 123 and the pulley 124 function as end tool second jaw pitch main pulleys. That is, the pulley 123 and the pulley 124 function as main rotation pulleys for a pitch motion of the second jaw 102. Here, the wire 306, which is a second jaw wire, is wound around the pulley 123, and the wire 302, which is a second jaw wire, is wound around the pulley 124.

The pulley 125 and the pulley 126 function as end tool second jaw sub-pulleys. That is, the pulley 125 and the pulley 126 function as sub rotation pulleys for a pitch motion of the second jaw 102. Here, the wire 306, which is a second jaw wire, is wound around the pulley 125, and the wire 302, which is a second jaw wire, is wound around the pulley 126.

On one side of the pulley 121, the pulley 123 and the pulley 124 are disposed to face each other. Here, the pulley 123 and the pulley 124 are formed to be rotatable independently of each other around the rotation shaft 143 that is an end tool pitch rotation shaft. In addition, the pulley 125 and the pulley 126 are disposed on one side of the pulley 123 and one side of the pulley 124, respectively, to face each other. Here, the pulley 125 and the pulley 126 are formed to be rotatable independently of each other around the rotation shaft 144, which is an end tool pitch auxiliary rotation shaft. Here, in the drawings, it is illustrated that all of the pulley 123, the pulley 125, the pulley 124, and the pulley 126 are formed to be rotatable around the Y-axis direction, but an embodiment of the present disclosure is not limited thereto, and the rotation axes of the respective pulleys may be formed in various directions according to configurations thereof.

The wire 306, which is a second jaw wire, is sequentially wound to make contact with at least portions of the pulley 125, the pulley 123, and the pulley 121. In addition, the wire 302 connected to the wire 306 by the second jaw wire coupling member 326 is sequentially wound to make contact with at least portions of the pulley 121, the pulley 122, the pulley 124, and the pulley 126.

Viewed from another perspective, the wires 306 and 302, which are second jaw wires, are sequentially wound to make contact with at least portions of the pulley 125, the pulley 123, the pulley 121, the pulley 122, the pulley 124, and the pulley 126, and are formed to move along the above pulleys while rotating the above pulleys.

Accordingly, when the wire 306 is pulled in the direction of an arrow of the wire 306 of FIGS. 14A to 14B, the second jaw wire coupling member 326 to which the wire 306 is coupled and the pulley 121 coupled to the second jaw wire coupling member 326 are rotated in the arrow R direction of FIGS. 14A to 14B. In contrast, when the wire 302 is pulled in the direction of an arrow of the wire 302 of FIG. 14A, the second jaw wire coupling member 326 to which the wire 302 is coupled and the pulley 121 coupled to the second jaw wire coupling member 326 are rotated in the arrow L direction of FIGS. 14A to 14B.

Hereinafter, a pitch motion of the present disclosure will be described in more detail.

First, for the pitch motion, at the end tool 100 side, the pulley 113, the pulley 114, the pulley 123, and the pulley 124, which are end tool jaw pitch main pulleys, are formed to be rotatable around the rotation shaft 143. Meanwhile, in a direction of the proximal end 105 of the end tool jaw pitch main pulley, the pulley 115, the pulley 116, the pulley 125, and the pulley 126, which are end tool jaw pitch sub-pulleys, are formed to be rotatable around the rotation shaft 144.

In addition, based on a plane perpendicular to the rotation shaft 141 and including the rotation shaft 143 (i.e., an XY plane), the wires 301 and 305, which are two strands of the first jaw wire, are located on the same side with respect to the XY plane. That is, the wire 301 and the wire 305 are formed to pass through lower sides of the pulley 113 and the pulley 114, which are end tool jaw pitch main pulleys, and upper sides of the pulley 115 and the pulley 116, which are end tool jaw pitch sub-pulleys.

Similarly, the wires 302 and 306, which are two strands of the second jaw wire, are located on the same side with respect to the XY plane. That is, the wires 302 and 306 are formed to pass through upper sides of the pulley 123 and the pulley 124, which are end tool jaw pitch main pulleys, and lower sides of the pulley 125 and the pulley 126, which are end tool jaw pitch sub-pulleys.

In addition, in the wires 301 and 305 that are two strands of the first jaw wire, when the wire 301 is pulled toward the arrow of the wire 301 of FIGS. 14A to 14B and simultaneously the wire 305 is pulled toward the arrow of the wire 305 of FIGS. 14A to 14B (i.e., when both strands of the first jaw wire are pulled in the same direction), as shown in FIG. 12, since the wires 301 and 305 are wound around lower portions of the pulleys 113 and 114, which are rotatable around the rotation shaft 143 that is an end tool pitch rotation shaft, the pulley 111 to which the wire 301 and the wire 305 are fixedly coupled, and the end tool hub 106 to which the pulley 111 is coupled are rotated together as a whole in a counterclockwise direction around the rotation shaft 143, as a result, the end tool 100 performs the pitch motion while rotating downward. At this time, since the second jaw 102 and the wires 302 and 306 fixedly coupled thereto are wound around the upper portions of the pulleys 123 and 124 rotatable around the rotation shaft 143, the wires 302 and 306 are unwound in opposite directions of the arrows of the wires 302 and 306, respectively.

In contrast, in the wires 302 and 306 that are two strands of the second jaw wire, when the wire 302 is pulled toward the arrow of the wire 302 of FIGS. 14A to 14B and simultaneously the wire 306 is pulled toward the arrow of the wire 306 of FIGS. 14A to 14B (i.e., when both strands of the second jaw wire are pulled in the same direction), as shown in FIG. 12, since the wires 302 and 306 are wound upward lower portions of the pulleys 123 and 124, which are rotatable around the rotation shaft 143 that is an end tool pitch rotation shaft, the pulley 121 to which the wire 302 and the wire 306 are fixedly coupled, and the end tool hub 106 to which the pulley 121 is coupled are rotated together as a whole in a clockwise direction around the rotation shaft 143. As a result, the end tool 100 performs the pitch motion while rotating upward. At this time, since the first jaw 101 and the wires 301 and 305 fixedly coupled thereto are wound downward the lower portions of the pulleys 113 and 114 rotatable around the rotation shaft 143, the wires 302 and 306 are moved in opposite directions of the arrows of the wires 301 and 305, respectively.

Viewed from another perspective, it may be also described that both strands of each jaw wire are moved simultaneously in the same direction when the end tool 100 is pitch-rotated.

Meanwhile, the end tool 100 of the surgical instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304 that are pitch wires. Specifically, the pulley 131 of the end tool 100 is rotatable around the rotation shaft 143, which is an end tool pitch rotation shaft, and may be integrally formed with the end tool hub 106 (or fixedly coupled to the end tool hub 106) as one body. In addition, the wires 303 and 304 may serve to connect the pulley 131 of the end tool 100 to the pulley 231 of the driving part 200.

Thus, when the pulley 231 of the driving part 200 is rotated, the rotation of the pulley 231 is transmitted to the pulley 131 of the end tool 100 via the wires 303 and 304, which causes the pulley 131 to also be rotated, and as a result, the end tool 100 performs a pitch motion while rotating.

That is, in the surgical instrument 30 according to an embodiment of the present disclosure, by providing the pulley 131 of the end tool 100, the pulley 231 of the driving part 200, and the wires 303 and 304 of the power transmission part 300 to transmit power for a pitch motion, the driving force for a pitch motion from the driving part 200 may be more completely transmitted to the end tool 100, thereby improving operation reliability.

Here, a diameter of each of the pulley 113, the pulley 114, the pulley 123, and the pulley 124, which are end tool jaw pitch main pulleys, and a diameter of the pulley 131, which is an end tool pitch pulley, may be the same as each other or different from each other. At this time, a ratio of the diameter of the end tool jaw pitch main pulley to the diameter of the end tool pitch pulley may be the same as a ratio of a diameter of a driving part relay pulley of the driving part 200, which will be described later, to a diameter of a driving part pitch pulley 231. This will be described in detail later.

Hereinafter, the driving part 200 of the surgical instrument 30 of FIG. 11 will be described in more detail.

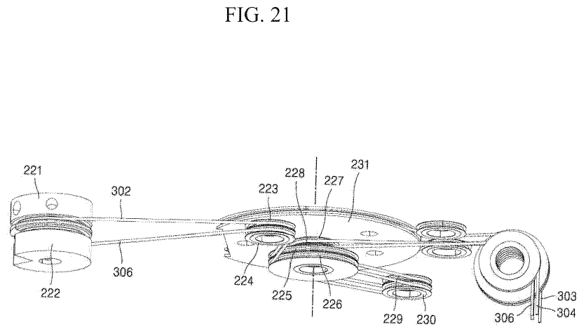

Referring to FIGS. 15 to 21, the driving part 200 of the surgical instrument 30 according to an embodiment of the present disclosure may include the pulley 211, the pulley 212, a pulley 213, a pulley 214, a pulley 215, a pulley 216, a pulley 217, a pulley 218, a pulley 219, and a pulley 220, which are related to a rotational motion of the first jaw 101. In addition, the driving part 200 may include the pulley 221, the pulley 222, a pulley 223, a pulley 224, a pulley 225, a pulley 226, a pulley 227, a pulley 228, a pulley 229, and a pulley 230, which are related to a rotational motion of the second jaw 102.

Here, the pulleys facing each other are illustrated in the drawings as being formed parallel to each other, but an embodiment of the present disclosure is not limited thereto, and each of the pulleys may be variously formed with a position and a size suitable for the configuration of the driving part.

In addition, the driving part 200 of the surgical instrument 30 according to an embodiment of the present disclosure may further include the pulley 231 serving as a driving part pitch pulley, and a pitch-yaw connector 232 configured to connect the pulley 231 to the above-described jaw pulleys of the driving part.

Further, the driving part 200 according to an embodiment of the present disclosure may include a rotation shaft 241, a rotation shaft 242, a rotation shaft 243, a rotation shaft 244, a rotation shaft 245, and a rotation shaft 246. Here, the rotation shaft 241 may function as a first jaw rotation shaft of the driving part, and the rotation shaft 242 may function as a second jaw rotation shaft of the driving part. In addition, the rotation shaft 243 may function as a driving part pitch rotation shaft, and the rotation shaft 244 may function as a driving part roll rotation shaft. In addition, the rotation shaft 245 may function as a driving part first jaw auxiliary rotation shaft of the driving part, and the rotation shaft 246 may function as a driving part second jaw auxiliary rotation shaft. Each of the rotation shafts 241, 242, 243, 244, 245, and 246 may be fitted into one or more pulleys, which will be described in detail later.

In addition, the driving part 200 according to an embodiment of the present disclosure may include a motor coupling part 251, a motor coupling part 252, a motor coupling part 253, and a motor coupling part 254. Here, the motor coupling part 251 may function as a first jaw driving motor coupling part, the motor coupling part 252 may function as a second jaw driving motor coupling part, the motor coupling part 253 may function as a pitch driving motor coupling part, and the motor coupling part 254 may function as a roll driving motor coupling part. Here, each of the motor coupling parts 251, 252, 253, and 254 may be provided in the form of a rotatable flat plate, in which one or more coupling holes, to which a motor (not shown) may be coupled, may be formed.

The motor coupling parts 251, 252, 253, and 254 of the driving part 200 described above are coupled to motors (not shown) formed in the robot arm units 21, 22, and 23, respectively, so that the driving part 200 is operated by driving the motors (not shown).

In addition, the driving part 200 according to an embodiment of the present disclosure may include a gear 261, a gear 262, a gear 263, and a gear 264. Here, the gear 261 and the gear 262 may function as pitch driving gears, and the gear 263 and the gear 264 may function as roll driving gears.

Hereinafter, each component will be described in more detail.

The pulley 211 and the pulley 212 may function as driving part first jaw pulleys, and the pulley 221 and the pulley 222 may function as driving part second jaw pulleys, and these components may be collectively referred to as driving part jaw pulleys.

Here, it is illustrated in the drawings that the pulley 211 is associated with a rotational motion of the first jaw 101 of the end tool 100, and the pulley 221 is associated with a rotational motion of the second jaw 102 of the end tool 100, but an embodiment of the present disclosure is not limited thereto. For example, one group of pulleys in the driving part may be associated with a yaw motion, and one group of pulleys in the driving part may be associated with an actuation motion. Thus, the pulley 211 and the pulley 212 may be collectively referred to as driving part driving pulleys. In addition, in the other pulleys, one group of pulleys may also be associated with a yaw motion, and one group of pulleys may also be associated with an actuation motion.

The pulley 213 and the pulley 214 may function as driving part first jaw auxiliary pulleys, and the pulley 223 and the pulley 224 may function as driving part second jaw auxiliary pulleys, and these components may be collectively referred to as driving part auxiliary pulleys.

The pulley 215 and the pulley 216 may function as driving part first jaw first relay pulleys, and the pulley 217 and the pulley 218 may function as driving part first jaw second relay pulleys, and these components may be collectively referred to as driving part first jaw relay pulleys. Meanwhile, the pulley 225 and the pulley 226 may function as driving part second jaw first relay pulleys, and the pulley 227 and the pulley 228 may function as driving part second jaw second relay pulleys, and these components may be collectively referred to as driving part second jaw relay pulleys. Meanwhile, the pulley 215, the pulley 216, the pulley 225, and the pulley 226 may be collectively referred to as driving part first relay pulleys, and the pulley 217, the pulley 218, the pulley 227, and the pulley 228 may be collectively referred to as driving part second relay pulleys. Furthermore, the pulley 215, the pulley 216, the pulley 217, the pulley 218, the pulley 225, the pulley 226, the pulley 227, and the pulley 228 may be collectively referred to as driving part relay pulleys.

Here, it is illustrated in the drawings that two pulleys are paired to form the driving part relay pulleys for each jaw, but an embodiment of the present disclosure is not limited thereto. For example, it is illustrated that the pulley 215, which is a driving part first jaw first relay pulley, and the pulley 217, which is a driving part first jaw second relay pulley, are formed as a pair, and the wire 301 sequentially passes through the pulley 215 and the pulley 217. However, the driving part first jaw relay pulley may be configured with not just two pulleys but also with three or more pulleys.

Meanwhile, the pulley 219 and the pulley 220 may function as driving part first jaw satellite pulleys, and the pulley 229 and the pulley 230 may function as driving part second jaw satellite pulleys, and these two components may be collectively referred to as driving part satellite pulleys.

A plurality of rotation shafts including the driving part first jaw rotation shaft 241, the driving part second jaw rotation shaft 242, the driving part pitch rotation shaft 243, the driving part roll rotation shaft 244, the driving part first jaw auxiliary rotation shaft 245, and the driving part second jaw auxiliary rotation shaft 246 may be formed on a first surface of a base plate 201. In addition, a plurality of relay pulleys 202 are formed on the first surface of the base plate 201, and may serve to redirect the wires 301, 302, 303, 304, 305, and 306 entering the driving part 200 through the connection part 310 toward the pulley 231.

Further, the connection part 310 in the form of a shaft is coupled to a second surface of the base plate 201 opposite to the first surface, and the first jaw motor coupling part 251, the second jaw driving motor coupling part 252, the pitch driving motor coupling part 253, and the roll driving motor coupling part 254, to which the motors (not shown) for driving the pulleys are coupled, may be formed on the second surface.

Here, each rotation shaft and each motor coupling part may be directly connected or indirectly connected to each other via a gear.

In an example, by directly coupling the first jaw motor coupling part 251 to the driving part first jaw rotation shaft 241, when the first jaw motor coupling part 251 coupled to a first jaw driving motor (not shown) is rotated, the driving part first jaw rotation shaft 241 directly coupled to the first jaw motor coupling part 251 may be rotated together. Similarly, by directly coupling the second jaw driving motor coupling part 252 to the driving part second jaw rotation shaft 242, when the second jaw driving motor coupling part 252 coupled to a second jaw driving motor (not shown) is rotated, the driving part second jaw rotation shaft 242 directly coupled to the second jaw driving motor coupling part 252 may be rotated together.

In another example, when viewed from a plane perpendicular to the driving part pitch rotation shaft 243, the pitch driving motor coupling part 253 and the driving part pitch rotation shaft 243 may be disposed to be spaced apart from each other by a certain extent. In addition, the pitch driving motor coupling part 253 and the driving part pitch rotation shaft 243 may be connected to each other by the gears 261 and 263, which are pitch driving gears.

Similarly, when viewed from a plane perpendicular to the driving part roll rotation shaft 244, the roll driving motor coupling part 254 and the driving part roll rotation shaft 244 may be disposed to be spaced apart from each other by a certain extent. In addition, the roll driving motor coupling part 254 and the driving part roll rotation shaft 244 may be connected to each other by the gears 263 and 264, which are roll driving gears.

As such, some motor coupling parts are configured to be directly connected to the rotation shafts, respectively, and the remaining motor coupling parts are configured to be indirectly connected to the rotation shafts, respectively, because the coupling position and direction between the surgical instrument 30 and the slave robot 20 should be considered. That is, the rotation shaft that is not affected by the coupling position with the slave robot 20 is directly connected to the motor coupling part, whereas the rotation shaft that may cause interference with the coupling position with the slave robot 20 may be indirectly connected to the motor coupling part.

It is illustrated in the drawings that the first jaw motor coupling part 251 and the second jaw driving motor coupling part 252 are directly connected to the rotation shafts, respectively, and the pitch driving motor coupling part 253 and the roll driving motor coupling part 254 are indirectly connected, respectively, through the gears, but an embodiment of the present disclosure is not limited thereto, and various configurations are possible according to the coupling position and direction with the slave robot 20.

The pulleys 211 and 212, which are driving part first jaw pulleys, may be coupled to the driving part first jaw rotation shaft 241. Here, the pulleys 211 and 212 may be formed to rotate together with the driving part first jaw rotation shaft 241.

In addition, the driving part first jaw auxiliary rotation shaft 245 may be disposed in a region adjacent to the driving part first jaw rotation shaft 241. The pulleys 213 and 214, which are driving part first jaw auxiliary pulleys, may be coupled to the driving part first jaw auxiliary rotation shaft 245. Here, the pulleys 213 and 214 may be formed to be rotatable around the driving part first jaw auxiliary rotation shaft 245.

Here, it is illustrated in the drawings that the driving part first jaw pulley is formed of two pulleys 211 and 212, the wire 301 is coupled to one pulley 211, and the wire 305 is coupled to the other pulley 212. However, an embodiment of the present disclosure is not limited thereto, and the driving part first jaw pulley may be formed of one pulley, and both the wires 301 and 305 may be coupled to the one pulley.

As described above, the driving part first jaw rotation shaft 241 is coupled to the first jaw driving motor (not shown) by the first jaw motor coupling part 251, and thus, when the first jaw driving motor (not shown) rotates for driving the first jaw 101, the pulleys 211 and 212, which are driving part first jaw pulleys, are rotated together with the driving part first jaw rotation shaft 241, so that the wires 301 and 305, which are first jaw wires, are pulled or released.

The pulleys 221 and 222, which are driving part second jaw rotation shafts, may be coupled to the driving part second jaw rotation shaft 242. Here, the pulley 221 and the pulley 222 may be formed to rotate together with the driving part second jaw rotation shaft 242.

In addition, the driving part second jaw auxiliary rotation shaft 246 may be disposed in a region adjacent to the driving part second jaw rotation shaft 242. The pulleys 223 and 224, which are driving part second jaw auxiliary pulleys, may be coupled to the driving part first jaw auxiliary rotation shaft 245. Here, the pulleys 223 and 224 may be formed to be rotatable around the driving part second jaw auxiliary rotation shaft 246.

Here, it is illustrated in the drawings that the driving part second jaw pulley is formed of two pulleys 221 and 222, the wire 302 is coupled to one pulley 221, and the wire 306 is coupled to the other pulley 222. However, an embodiment of the present disclosure is not limited thereto, and the driving part second jaw pulley may be formed of one pulley, and both the wires 302 and 306 may be coupled to the one pulley.

As described above, the driving part second jaw rotation shaft 242 is coupled to the second jaw driving motor (not shown) by the second jaw driving motor coupling part 252, and thus, when the second jaw driving motor (not shown) rotates for driving the second jaw 102, the pulley 221 and the pulley 222, which are driving part second jaw pulleys, are rotated together with the driving part second jaw rotation shaft 242, so that the wires 302 and 306, which are second jaw wires, are pulled or released.

The pulley 231, which is a driving part pitch pulley, may be coupled to the driving part pitch rotation shaft 243. Here, the pulley 231 may be formed to rotate together with the driving part pitch rotation shaft 243.

As described above, the driving part pitch rotation shaft 243 is coupled to a pitch driving motor (not shown) by the pitch driving motor coupling part 253, and thus, when the pitch driving motor (not shown) rotates for a pitch motion, the wires 303 and 304, which are pitch wires, are pulled or released as the pulley 231, which is a driving part pitch pulley, is rotated together with the driving part pitch rotation shaft 243.

Meanwhile, the pulley 215, the pulley 216, the pulley 217, the pulley 218, the pulley 225, the pulley 226, the pulley 227, and the pulley 228, which are driving part relay pulleys, may be formed to be rotatable around the driving part pitch rotation shaft 243 by inserting the driving part pitch rotation shaft 243 therethrough. Here, the pulley 215, the pulley 216, the pulley 217, and the pulley 218, which are driving part first jaw relay pulleys, may be disposed on one surface side of the pulley 231 that is a pitch pulley, and the pulley 225, the pulley 226, the pulley 227, and the pulley 228, which are driving part second jaw relay pulleys, may be disposed on the other surface side of the pulley 231.

Viewed from another perspective, along the driving part pitch rotation shaft 243, the pulleys 225 and 226, which are driving part second jaw first relay pulleys, the pulleys 227 and 228, which are driving part second jaw second relay pulleys, the pulley 231, which is a driving part pitch pulley, and the pulleys 217 and 218, which are driving part first jaw second relay pulleys, and the pulleys 215 and 216, which are driving part first jaw first relay pulleys, are sequentially stacked and formed.

In addition, the pitch-yaw connector 232 may be coupled to the driving part pitch rotation shaft 243. The pitch-yaw connector 232 may be formed to rigidly connect the pulley 231, which is a driving part pitch pulley, to the pulley 219, the pulley 220, the pulley 229, and the pulley 230, which are driving part satellite pulleys to allow the driving part satellite pulleys to be revolved around the driving part pitch rotation shaft 243 when the pulley 231 is rotated. This will be described in detail later.

Here, the pitch-yaw connector 232 may be formed to rotate together with the driving part pitch rotation shaft 243. That is, the pulley 231 and the pitch-yaw connector 232 may be coupled to the driving part pitch rotation shaft 243, and may be rotated together with the driving part pitch rotation shaft 243.

Here, the pitch-yaw connector 232 may be described as being formed in an approximately Y-shape as shown in FIG. 17, or the pitch-yaw connector 232 may be described as being formed in a shape in which at least two extension portions 232a and 232b are formed to extend from the center thereof. In addition, a driving part first jaw satellite pulley central shaft 233 and a driving part second jaw satellite pulley central shaft 234 may be formed at end portions of the extension portions 232a and 232b, respectively.

In addition, the pulleys 219 and 220, which are driving part first jaw satellite pulleys, may be coupled to the driving part first jaw satellite pulley central shaft 233, and the pulleys 229 and 230, which are driving part second jaw satellite pulleys, may be coupled to the driving part second jaw satellite pulley central shaft 234.

As a result, when the pulley 231, which is a driving part pitch pulley, is rotated together with the driving part pitch rotation shaft 243, the pulley 219, the pulley 220, the pulley 229, and the pulley 230, which are driving part satellite pulleys, are revolved around the driving part pitch rotation shaft 243. In other words, it may be said that the driving part first jaw satellite pulley central shaft 233 and the driving part second jaw satellite pulley central shaft 234 are rotated around the driving part pitch rotation shaft 243 while maintaining a constant distance from the driving part pitch rotation shaft 243 in a state in which the driving part first jaw satellite pulley central shaft 233 and the driving part second jaw satellite pulley central shaft 234 are spaced apart from the driving part pitch rotation shaft 243 by a certain extent.

That is, the driving part satellite pulley is formed to be movable relative to the driving part relay pulley and the driving part pitch rotation shaft 243 so that a relative position of the driving part satellite pulley with respect to the driving part relay pulley and the driving part pitch rotation shaft 243 may be changed. On the other hand, the relative positions of the driving part pitch pulley 231 and the driving part relay pulley remain constant.

In addition, when the pulley 231, which is a driving part pitch pulley, is rotated around the driving part pitch rotation shaft 243, the pulley 219, the pulley 220, the pulley 229, and the pulley 230, which are driving part satellite pulleys, are moved relative to the pulley 231, which is a driving part pitch pulley, so that the overall lengths of the wire 301, the wire 302, the wire 305, and the wire 306, which are jaw wires, in the driving part 200 are changed.

The wire 301, which is a first jaw wire, is connected to the end tool 100 through the connection part 310 after being sequentially wound to make contact with at least portions of the pulley 211, the pulley 213, the pulley 215, the pulley 219, and the pulley 217 in a state in which one end portion of the wire 301 is coupled to the pulley 211 by the first jaw wire-driving part coupling member (not shown).

Viewed from another perspective, the wire 301, which is a first jaw wire, is connected to the end tool 100 through the connection part 310 after being sequentially passing through the driving part first jaw pulley 211, the driving part first jaw auxiliary pulley 213, the driving part first jaw first relay pulley 215, the driving part first jaw satellite pulley 219, and the driving part first jaw second relay pulley 217.

Viewed from another perspective, the wire 301, which is a first jaw wire, enters the driving part 200 after passing through the end tool 100 and the connection part 310, and then is fixedly coupled to the pulley 211, which is a driving part first jaw pulley after being sequentially wound around the pulley 217, the pulley 219, the pulley 215, and the pulley 213.

Meanwhile, the wire 305, which is a first jaw wire, is connected to the end tool 100 through the connection part 310 after being sequentially wound to make contact with at least portions of the pulley 212, the pulley 214, the pulley 216, the pulley 220, and the pulley 218 in a state in which one end portion of the wire 305 is coupled to the pulley 212 by the first jaw wire-driving part coupling member (not shown).

The wire 302, which is a second jaw wire, is connected to the end tool 100 through the connection part 310 after being sequentially wound to make contact with at least portions of the pulley 221, the pulley 223, the pulley 225, the pulley 229, and the pulley 227 in a state in which one end portion thereof is coupled to the pulley 221 by the second jaw wire-driving part coupling member (not shown).

Meanwhile, the wire 306, which is a second jaw wire, is connected to the end tool 100 through the connection part 310 after being sequentially wound to make contact with at least portions of the pulley 222, the pulley 224, the pulley 226, the pulley 230, and the pulley 228 in a state in which one end portion thereof is coupled to the pulley 222 by the second jaw wire-driving part coupling member (not shown).

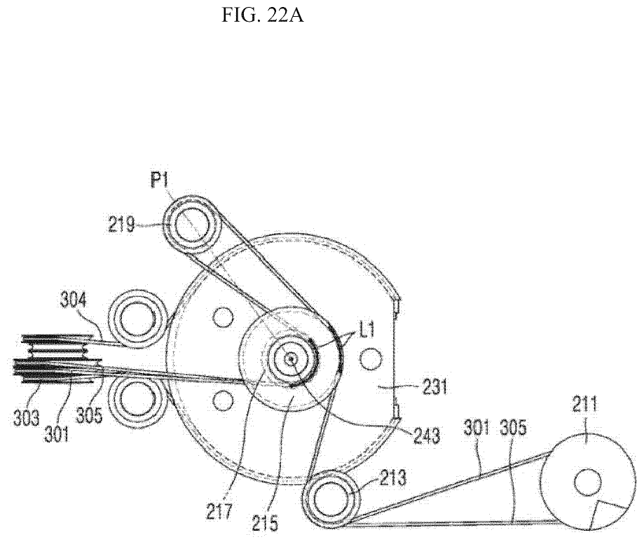

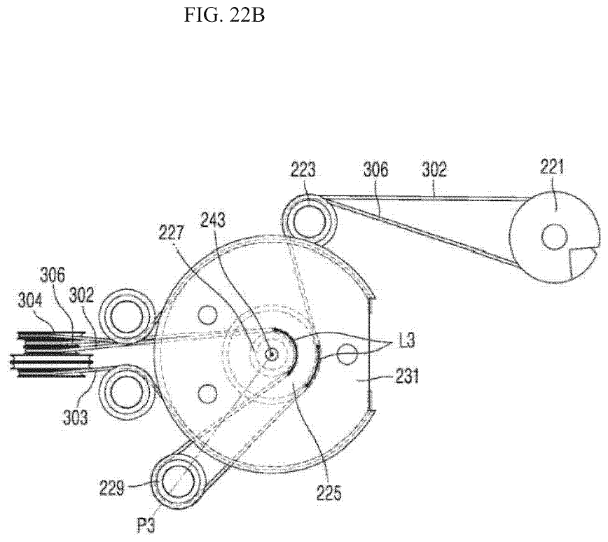

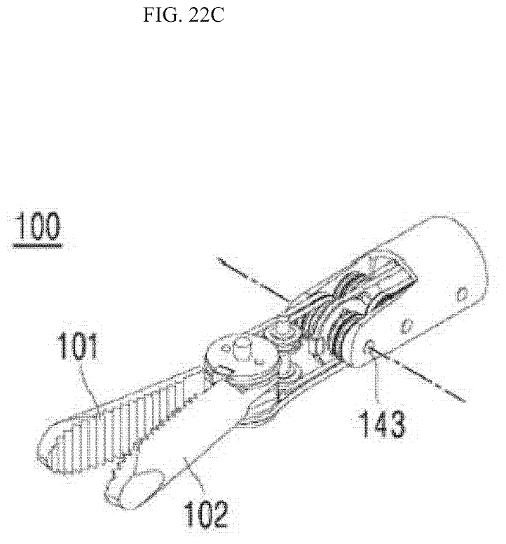

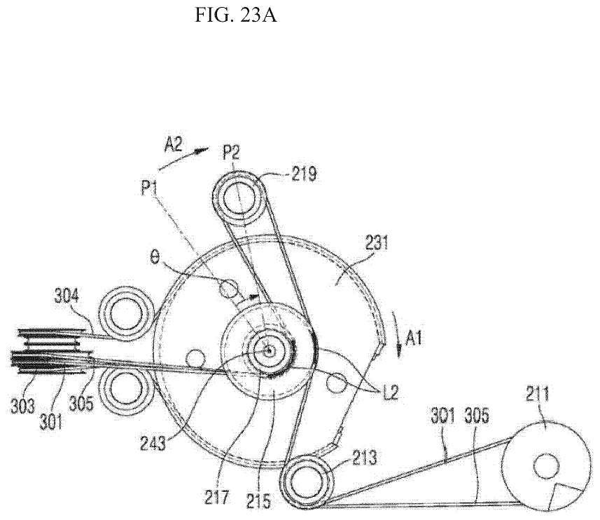

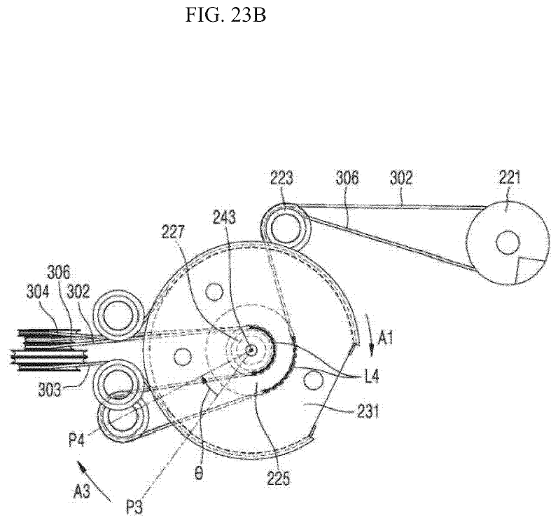

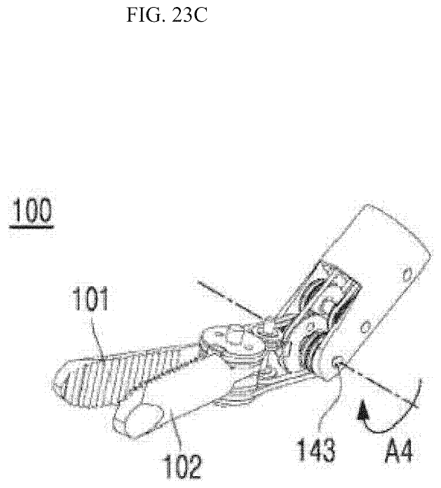

FIGS. 22A to 23C are diagrams illustrating a pitch motion of the surgical instrument illustrated in FIG. 11. Here, for convenience of description, only the pulleys and wires related to the rotation of the first jaw are illustrated in FIG. 22A and FIG. 23A, and only the pulleys and wires related to the rotation of the second jaw are illustrated in FIG. 22B and FIG. 23B. In addition, FIG. 22C and FIG. 23C illustrate a pitch motion of the end tool according to a pitch motion of the driving part.

Here, in the surgical instrument 30 according to an embodiment of the present disclosure, when the driving part satellite pulley is moved relative to the driving part relay pulley, which causes the overall length of the jaw wire to be changed in the driving part 200, allowing the end tool 100 to perform a pitch motion. In particular, in the surgical instrument 30 according to an embodiment of the present disclosure, when the driving part pitch pulley 231 is rotated, which causes the driving part satellite pulley to be revolved around the (common) rotation shaft of the driving part relay pulley and the driving part pitch pulley 231 so that a path length of the jaw wire wound around the driving part relay pulley is changed, allowing the end tool to perform a pitch motion.

Specifically, when a motion compensation for the pitch motion is not separately performed in the driving part, the pitch motion itself cannot be performed in the end tool.

Meanwhile, in order for the end tool to perform a pitch motion, the wires 301 and 305 should be further wound around the pulley 113 by ΔSpitch and the wires 302 and 306 should be further unwound from the pulley 114 by ΔSpitch. However, when such compensation is not performed in the driving part, the pitch motion itself cannot be performed in the end tool.

In order to perform motion compensation for the pitch motion as described above, in the surgical instrument 30 according to an embodiment of the present disclosure, the driving part pitch pulleys are rotated while the driving part satellite pulleys are revolved, so that the jaw wires are wound around or released from the driving part relay pulley, which allows the movement of the jaw wires to be compensated for by the rotation of the driving part pitch pulley 231.

In other words, when the pulley 231, which is a driving part pitch pulley, is rotated together with the driving part pitch rotation shaft 243, the driving part satellite pulleys are revolved around the driving part pitch rotation shaft 243. In addition, as the driving part satellite pulleys are revolved around the driving part pitch rotation shaft 243, the jaw wire wound around the driving part relay pulley is changed in length. That is, the jaw wire wound at the end tool 100 side due to the rotation of the pulley 231 is released by the same amount at the driving part 200 side, and the jaw wire unwound at the end tool 100 side is wound by the same amount at the driving part 200 side, so that the pitch motion does not affect the yaw motion.

Viewed from another perspective, when the end tool performs a pitch motion due to the rotation of the driving part pitch pulley 231, the jaw wire (responsible for the yaw and actuation motions) is also moved by the pitch motion. That is, as the pitch rotation is performed around the rotation shaft 143 of the end tool 100, both strands of the jaw wire coupled to one jaw are pulled, and both strands thereof coupled to the other jaw are released. Accordingly, it may be described that in the present disclosure, in order to compensate for the movement of the jaw wire, when the end tool performs the pitch motion, the overall length of the jaw wire in the driving part is changed while the driving part satellite pulley is moved relative to the driving part relay pulley, so that the jaw wire is released (or pulled) at the end tool side as much as the jaw wire is pulled (or released) at the driving part side, thereby compensating for the movement of the jaw wire when the end tool performs the pitch motion.

Hereinafter, the pitch motion will be described in more detail.

When the pulley 231, which is a driving part pitch pulley, is rotated in the direction of an arrow A1 (i.e., in the clockwise direction in the drawing) in order for the pitch motion, the pitch-yaw connector 232 (see FIG. 15) is rotated in the direction of the arrow A1 together with the pulley 231, and thus, the pulleys 219 and 220, which are driving part satellite pulleys fixedly coupled to the pitch-yaw connector 232 (see FIG. 15), are revolved as a whole in the direction of an arrow A2 of FIG. 23A (i.e., in the clockwise direction in the drawing) around the driving part pitch rotation shaft 243 by θ. That is, when the pulley 231 is rotated, the pulleys 219 and 220 are revolved by θ from the position of P1 of FIG. 22A to the position of P2 of FIG. 23A. Viewed from another perspective, it may be described that when the driving part pitch pulley 231 is rotated, the driving part satellite pulley is moved in conjunction with the driving part pitch pulley 231.

At the same time, when the pulley 231, which is a driving part pitch pulley, is rotated in the direction of the arrow A1 (i.e., in the clockwise direction in the drawing), the pitch-yaw connector 232 (see FIG. 15) is rotated in the direction of the arrow A1 together with the pulley 231, and thus, the pulleys 229 and 230, which are driving part satellite pulleys fixedly coupled to the pitch-yaw connector 232 (see FIG. 15), are revolved as a whole in the direction of an arrow A3 of FIG. 23B (i.e., in the clockwise direction in the drawing) around the driving part pitch rotation shaft 243 by θ. That is, when the pulley 231 is rotated, the pulleys 229 and 230 are revolved by θ from the position of P3 of FIG. 22B to the position of P4 of FIG. 23B. Viewed from another perspective, it may be described that when the driving part pitch pulley 231 is rotated, the driving part satellite pulley is moved in conjunction with the driving part pitch pulley 231.

Meanwhile, in this case, the positions of the pulley 215, the pulley 216, the pulley 217, the pulley 218, the pulley 225, the pulley 226, the pulley 227, and the pulley 228, which are driving part relay pulleys coupled to the driving part pitch rotation shaft 243, are not changed. That is, the relative positions of the pulley 211, which is a driving part jaw pulley, the pulley 231, which is a driving part pitch pulley, and the pulley 215, the pulley 216, the pulley 217, and the pulley 218, which are driving part relay pulleys, remain constant. Similarly, the relative positions of the pulley 221, which is a driving part jaw pulley, the pulley 231, which is a driving part pitch pulley, and the pulley 225, the pulley 226, the pulley 227, and the pulley 228, which are driving part relay pulleys, remain constant.

In addition, as described above, the relative position of the driving part satellite pulley with respect to the driving part relay pulley is changed as the driving part satellite pulley is revolved, and thus, the length of each wire wound around the driving part relay pulley, that is, the path length, is changed. Here, since the driving part relay pulley includes the pulley 215, which is a driving part first jaw first relay pulley, and the pulley 217, which is a driving part first jaw second relay pulley, the path length also means the sum of the length of the wire 301 wound around the pulley 215 and the length of the wire 301 wound around the pulley 217 (or, the sum of the length by which the wire 305 is wound around the pulley 216 and the length by which the wire 305 is wound on the pulley 218).

That is, as compared to a path length L1 by which the wires 301 and 305, which are first jaw wires, wound around the driving part relay pulleys at the position of FIG. 22A, a path length L2 by which the first jaw wires wound around the driving part relay pulleys at the position of FIG. 23A is reduced, and thus, the first jaw wires are further released at the driving part 200 side by the reduced path length (L1-L2). That is, the overall lengths of the wires 301 and 305, which are first jaw wires, in the driving part 200 are reduced. In addition, as the overall length of the first jaw wire in the driving part 200 is reduced, the overall length of the first jaw wire in the end tool 100 is increased as much as the first jaw wire is unwound.

In contrast, when the pulley 231, which is a driving part pitch pulley, is rotated in the direction of the arrow A1, as compared to a path length L3 by which the wires 302 and 306, which are second jaw wires, wound around the driving part relay pulleys at the position of FIG. 22B, a path length L4 by which the second jaw wires wound around the driving part relay pulleys at the position of FIG. 23B is increased, and the second jaw wires are further pulled at the driving part 200 side by as much as the increased path length (L4-L3). That is, the overall lengths of the wires 302 and 306, which are second jaw wires, in the driving part 200 are increased. In addition, as the overall length of the second jaw wire in the driving part 200 is increased, the overall length of the second jaw wire in the end tool 100 is reduced as much as the second jaw wire is pulled.

As such, when the pulley 231, which is a driving part pitch pulley, is rotated in the direction of the arrow A1 for a pitch motion, the relative position of the driving part satellite pulley is changed as the driving part satellite pulley is moved relative to the driving part pitch pulley 231 and the driving part relay pulley. In addition, due to the relative movement of the driving part satellite pulley, the overall length of the first jaw wire in the driving part 200 is reduced, and the overall length of the first jaw wire in the end tool 100 is increased. At the same time, due to the relative movement of the driving part satellite pulley, the overall length of the second jaw wire in the driving part 200 is increased, and the overall length of the second jaw wire in the end tool 100 is reduced.

As a result, when the pulley 231, which is a driving part pitch pulley, is rotated in the direction of the arrow A1, the wires 301 and 305, which are two strands of the first jaw wire, are released and the wires 302 and 306, which are two strands of the second jaw wire, are pulled when viewed from the end tool 100 side, so that the end tool 100 performs a pitch motion in the direction of an arrow A4 around the rotation shaft 143.

Here, the term “path length” may be defined as a length of the jaw wire from a point at which the jaw wire enters the driving part first relay pulley to a point at which the jaw wire exits from the driving part second relay pulley through the driving part satellite pulley. That is, the path length may be defined as a length of the wire 301, which is a jaw wire, from a point at which the jaw wire enters the pulley 215, which is a driving part first relay pulley, to a point at which the jaw wire exits from the pulley 217, which is a driving part second relay pulley, through the pulley 219 that is a driving part satellite pulley.

Viewed from another perspective, the path length may be defined as the length of the jaw wire from an initial contact point of the jaw wire with the driving part relay pulley to a final contact point of the jaw wire with the driving part relay pulley on a deployment path of the jaw wire that connects the end tool jaw pulley to the driving part jaw pulley. That is, the path length may be defined as the length of the jaw wire from an initial contact point of the wire 301, which is a jaw wire, with the pulley 215, which is a driving part first relay pulley, to a final contact point of the wire 301 with the pulley 217, which is a driving part second relay pulley.

Meanwhile, as the above-described path length is changed while the driving part satellite pulley is moved relative to the driving part relay pulley, the overall length of the jaw wire in the driving part 200 is also changed. In addition, as the overall length of the jaw wire in the driving part 200 is changed, the overall length of the jaw wire in the end tool 100 is also changed. However, it may be said that since the overall length of the jaw wire in the end tool 100 is also increased (or reduced) by as much as the overall length of the jaw wire increased (reduced) in the driving part 200, a total length of the jaw wire is not changed (assuming that elastic deformation or the like is not considered).

As a result, when the driving part pitch pulley 231 is rotated, the wire 301/wire 305, which are first jaw wires, are released at the driving part 200 side by as much as the wire 301/wire 305, which are first jaw wires, are pulled at the end tool 100 side, as a result, a pitch motion is enabled.

Meanwhile, as described above, the end tool 100 of the surgical instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304 which are pitch wires.

Accordingly, when the pulley 231, which is a driving part pitch pulley, is rotated in the direction of the arrow A1, due to the rotation of the pulley 231, the wire 304 is wound around the pulley 231 and the wire 303 is released from the pulley 231. Accordingly, the pulley 131, which is an end tool pitch pulley connected to the other sides of the wires 303 and 304, is rotated in the direction of the arrow A2 around the rotation shaft 143, so that the pitch motion may be more surely and reliably performed.

Here, among the pulleys that are rotated around the rotation shaft 143, which is an end tool pitch rotation shaft, the pulley 131, which is an end tool pitch pulley in contact with the wires 303 and 304 that are pitch wires, may be formed to have a diameter different from those of the pulley 113, the pulley 114, the pulley 123, and the pulley 124, which are end tool jaw pitch main pulleys in contact with the wire 301, the wire 305, the wire 302, and the wire 306 that are jaw wires.

In this case, when the rotation shaft 143 is rotated, the lengths of the wires wound around or unwound from the respective pulleys are different from each other. For example, when a diameter of the end tool pitch pulley is 6 φ, a diameter of the end tool jaw pitch main pulley is 4 φ, and the rotation shaft 143 is rotated by 90°, a length of the pitch wire wound around the end tool pitch pulley is 1.5 π, whereas a length of the jaw wire wound around the end tool jaw pitch main pulley may be 1 π.

From this perspective, the length of the wire wound around or unwound from the pulley may be defined as “rotation amount”. The rotation amount is a concept different from a rotation angle, and may be calculated as (diameter*rotation angle/360°*π).

In this case, since essentially the pulley 231, which is a driving part pitch pulley, is directly connected to the pulley 131, which is an end tool pitch pulley, by the wires 303 and 304, which are pitch wires, the rotation amount of the driving part pitch pulley 231 is the same as that of the end tool pitch pulley. That is, the pitch wire is released from or wound around the end tool pitch pulley by as much as the pitch wire is wound around or released from the driving part pitch pulley 231.

Meanwhile, a relation of (diameter of end tool pitch pulley: diameter of end tool jaw pitch main pulley)=(rotation amount of wire wound around end tool pitch pulley: rotation amount of wire wound around end tool jaw pitch main pulley) may be established.

As described above, when, in the end tool 100, the length of the pitch wire wound around the end tool pitch pulley is different from the length of the jaw wire wound around the end tool jaw pitch main pulley, in the driving part 200, the length of the pitch wire to be released should be different from the length of the jaw wire to be released by the same proportion.

To this end, the relationship of (diameter of end tool pitch pulley: diameter of end tool jaw pitch main pulley)=(diameter of driving part pitch pulley: diameter of driving part relay pulley) may be established.

For example, when a ratio of (diameter of end tool pitch pulley: diameter of end tool jaw pitch main pulley) is 6:4, a ratio of (diameter of driving part pitch pulley: diameter of driving part relay pulley) may also be 11:4. According to this ratio, the diameter of the driving part pitch pulley may be 9 φ, and the diameter of the driving part relay pulley may be 6 φ.

However, here, the driving part relay pulley may include two or more pulleys including the driving part first relay pulley and the driving part second relay pulley. In addition, the sum of the diameters of the driving part first relay pulley and the driving part second relay pulley may be defined as the diameter of the driving part relay pulley.

For example, when the diameter of the driving part relay pulley is 6φ, there are several possible combinations for (diameter of driving part first relay pulley, diameter of driving part second relay pulley), including (1φ, 5φ), (2φ, 4φ), (3φ, 3φ), (4φ, 2φ), and (5φ, 1φ), among others. Here, it is illustrated in the drawings that the diameter of the pulley 215, which is a driving part first relay pulley, is 4 φ, and the diameter of the pulley 217, which is the driving part second relay pulley, is 2 φ.

In addition, it may be described that rotation amount of driving part first relay pulley plus the rotation amount of driving part second relay pulley is proportional to the rotation amount of the driving part pitch pulley.

However, although the ratio of (diameter of end tool pitch pulley: diameter of end tool jaw pitch main pulley) may not exactly match the ratio of (diameter of driving part pitch pulley: diameter of driving part relay pulley), when the pulley diameters are selected to make these ratios similar, the object of the present disclosure, which is to compensate for the movement of the jaw wire with the rotation of the driving part pitch pulley, can be achieved to some extent.

The process of the final pitch motion will be described again as follows.

Hereinafter, a case in which the diameter of the end tool pitch pulley is 6 φ, the diameter of the end tool jaw pitch main pulley is 4 φ, the diameter of the driving part pitch pulley is 9 φ, and the diameter of the driving part relay pulley is 6 φ will be described as an example.

First, for a pitch motion, the pulley 231, which is a driving part pitch pulley of the driving part 200, is rotated by 60° to wind the wire 304, which is a pitch wire, while releasing the wire 303. At this time, the length of the wire 303/wire 304 wound and unwound is 1.5 π.

Accordingly, as the wire 304 is pulled by 1.5 π and the wire 303 is released by 1.5 π in the end tool 100, the pulley 131, which is an end tool pitch pulley, is rotated by 90° corresponding to 1.5 π.

Meanwhile, when the pulley 131 is pitch-rotated around the rotation shaft 143, the jaws 101 and 102 and the pulley 111/pulley 112 are also pitch-rotated around the rotation shaft 143. Accordingly, the wires 301 and 305, which are first jaw wires coupled to the pulley 111, are both pulled, and the wires 302 and 306, which are second jaw wires coupled to the pulley 121, are both released. At this time, the angles by which the end tool pitch pulley and the end tool jaw pitch main pulley are rotated are equal to each other and measure 90°, and thus, the length of the jaw wires wound around or released from the end tool jaw pitch main pulley becomes 1 π.

Meanwhile, since the pulley 231 and the pulley 219/pulley 220 are rigidly connected by the pitch-yaw connector 232, when the pulley 231 is rotated by 60° around the driving part pitch rotation shaft 243, the pulley 219/pulley 220 are revolved by 60° around the driving part pitch rotation shaft 243.

In addition, as described above, as the pulley 219/pulley 220 are revolved, the jaw wires are wound around or released from the pulley 215 and the pulley 216, whose combined diameter is 6 φ, by 1 π corresponding to a revolution angle of 60°. That is, the wires 301 and 305, which are first jaw wires, are released as a whole, and the wires 302 and 306, which are second jaw wires, are pulled as a whole.

In other words, the overall path lengths of the wires 301 and 305 wound around the pulley 215, the pulley 216, the pulley 217, and the pulley 218, which are driving part first jaw relay pulleys, are reduced, and the wires 301 and 305 are released by as much as the reduced path length. In addition, the overall path lengths of the wires 302 and 306 wound around the pulley 225, the pulley 226, the pulley 227, and the pulley 228, which are driving part second jaw relay pulleys, are increased, and the wires 302 and 306 are pulled by as much as the increased path length.

That is, the wires 301 and 305, which are first jaw wires, are released at the driving part 200 side by as much as the wires 301 and 305 are pulled at the end tool 100 side, thereby compensating for the movement of the jaw wire due to the pitch motion. Similarly, the wires 302 and 306, which are second jaw wires, are released at the driving part 200 side by as much as the wires 302 and 306 are pulled at the end tool 100 side, thereby compensating for the movement of the jaw wire due to the pitch motion.

As a result, by releasing (or pulling) the jaw wires at the driving part 200 side by as much as a length equal to the length by which the jaw wires are wound around (or released from) the end tool 100 side in response to the pitch motion, the pitch motion can be performed independently without affecting the rotation of the jaw around the yaw shaft.

That is, when the driving part pitch pulley 231 and the driving part satellite pulley are rigidly connected, and the driving part pitch pulley 231 is rotated around the driving part pitch rotation shaft 243, the path length of the jaw wire wound around the driving part relay pulley is changed as the driving part satellite pulley is revolved around the driving part pitch rotation shaft 243. In addition, the change in the path length of the jaw wire compensates for the movement of the jaw wires at the end tool side due to the pitch motion, as a result, the pitch motion is independently performed.

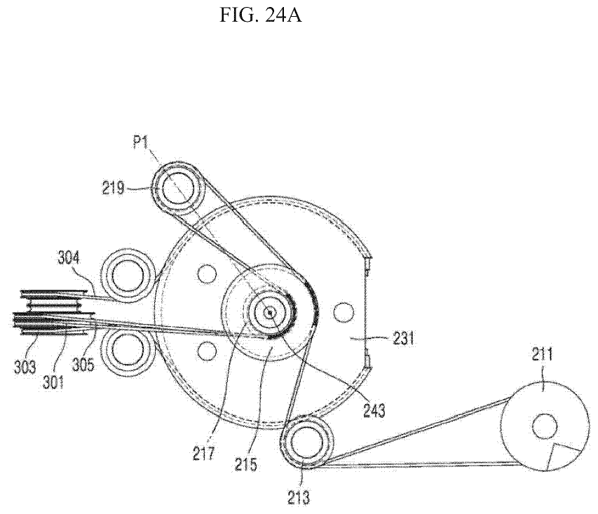

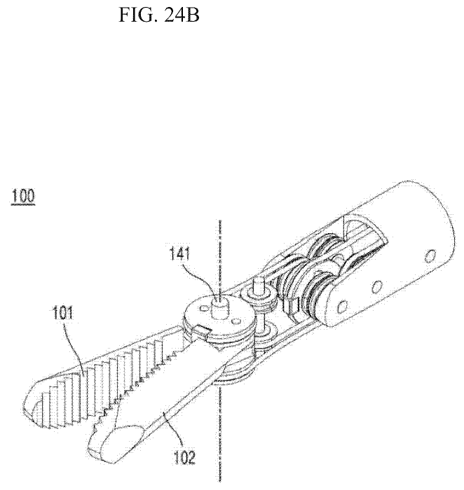

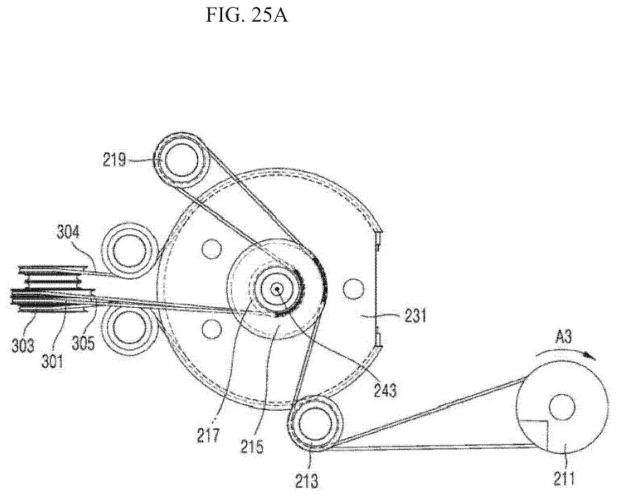

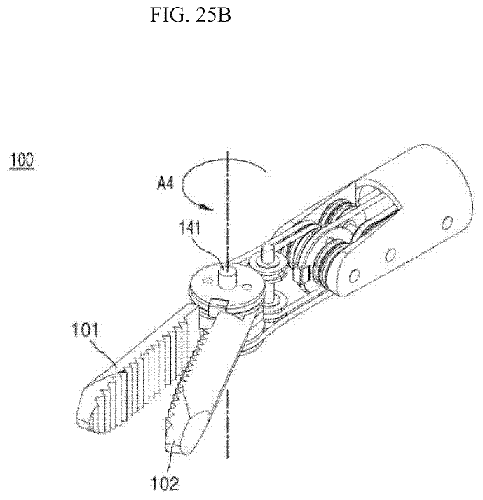

FIGS. 24A to 25B are diagrams illustrating a yaw motion of the surgical instrument illustrated in FIG. 11.

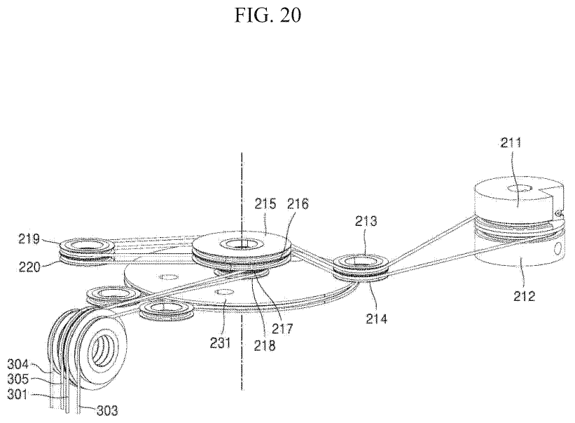

Referring to FIGS. 20, 21, 24A to 25B and the like, when the pulley 211, which is a driving part first jaw pulley, is rotated in the direction of an arrow A3 for a yaw motion, one of the wires 301 and 305, which are first jaw wires, is wound around the pulley 211 and the other one thereof is released from the pulley 211 in response to the rotation of the pulley 211. Accordingly, the pulley 111, which is an end tool first jaw pulley connected to the opposite side of the wires 301 and 305, is rotated in the direction of as arrow A4, so that the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wires 301 and 305 are wound around or released from the driving part satellite pulley and the driving part relay pulley occurs.

Accordingly, the driving part pitch pulley 231 rigidly connected to the driving part satellite pulley is not rotated, and the wires 303 and 304, which are pitch wires, are not wound or released and maintained in position.

Similarly, when the pulley 221, which is a driving part second jaw pulley, is rotated for a yaw motion, in response to the rotation of the pulley 221, one of the wires 302 and 306, which are second jaw wires, is wound around the pulley 221 and the other one thereof is released from the pulley 221. Accordingly, the pulley 121, which is an end tool second jaw pulley connected to the opposite side of the wires 302 and 306, is rotated in one direction, so that the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wires 302 and 306 are wound around or released from the driving part satellite pulley and the driving part relay pulley occurs.

Accordingly, the driving part pitch pulley 231 rigidly connected to the driving part satellite pulley is not rotated, and the wires 303 and 304, which are pitch wires, are not wound or released and maintained in position.

As a result, the overall lengths of the wire 301, the wire 302, the wire 305, and the wire 306, which are jaw wires, in the driving part 200 remain constant even when the pulley 211 or pulley 221, which is a driving part jaw pulley, is rotated for the yaw or actuation motion.

As described above, in the surgical instrument 30 according to an embodiment of the present disclosure, when the driving part pitch pulley 231 is rotated, the driving part satellite pulley is revolved around the rotation shaft of the driving part pitch pulley 231 to change the path length of the jaw wire wound around the driving part relay pulley, and the jaw wire is wound or released in response to the rotation of the driving part pitch pulley 231, so that the movement of the jaw wire due to the pitch drive may be offset or compensated, and as a result, the effect of separating the pitch motion and the yaw motion can be obtained.

However, the pitch motion and the yaw motion are not limited to being mechanically separated from each other as described above, and can be separated and performed independently by the processor according to an embodiment of the present disclosure.

Hereinafter, a surgical robot system including at least one driving element having an elastic joint and a processor configured to control the same according to aspects of the present disclosure will be described more specifically, but not limitedly. According to an aspect, the surgical robot system includes the at least one driving element, wherein the driving element may include a first member, a second member coupled so as to be capable of changing its relative position with respect to a first member, and at least one joint member providing a rotational coupling between the first member and the second member. Herein, the at least one joint member may be an elastic joint member that allows twisting between members that are targets of rotational coupling due to an external force. In the following, for the convenience of explanation, a non-limiting example in which the driving element provided in the surgical robot system is a robot arm will be first described, but it should be noted that the technical idea of the present disclosure is not limited thereto. It should be understood that the driving element provided in the surgical robot system includes all arbitrary configurations that generate movement through a connection relationship between a specific member and another member.

### Remote Center of Motion

As previously discussed, when a surgery is performed using a surgical robot system, the robot arm performs the surgery under the control of a surgeon. In this connection, a surgical instrument operates inside the body of a patient, and a plurality of structures including a robot arm for controlling the same from outside the body of the patient are disposed. The surgical instrument used for laparoscopic surgery is inserted into the body of the patient through at least one port formed in the body. In order to prevent damage to the body of the patient, the surgical robot is required to operate while maintaining such a port as a remote center of motion. In other words, even when the robot arm and/or the surgical instrument of the surgical robot operates, the point corresponding to the port formed in the body of the patient needs to be controlled in a fixed state.

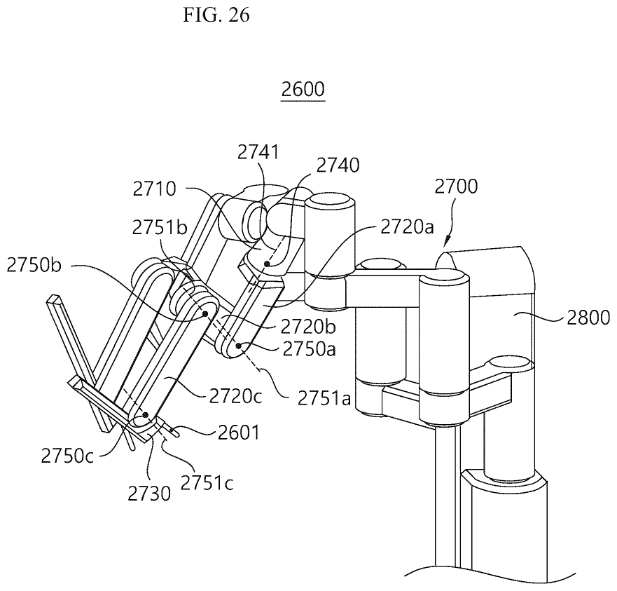

In this regard, FIG. 26 is a perspective view of a surgical robot system having an elastic joint according to an embodiment of the present disclosure. As illustrated in FIG. 26, a surgical robot 2600 according to an aspect of the present disclosure may include a main body 2800 and at least one robot arm 2700 connected to the base unit. As exemplarily illustrated in FIG. 26, one surgical robot 2600 may be provided with a plurality of robot arms, but is not limited thereto. The surgical robot system may include a plurality of surgical robots each having one robot arm, or may include a single surgical robot having a plurality of robot arms. In addition, for example, both a surgical robot having a plurality of robot arms and a surgical robot having one robot arm may be provided.

The surgical robot system according to an embodiment of the present disclosure may include at least one surgical robot arm 2700 and a processor configured to control the surgical robot arm. Herein, the processor may be provided in a master device, as described above in the present disclosure, or may be provided in a slave robot, or may be provided in a separate device and configured to transmit and receive information with the master device and/or the slave robot.

According to an aspect, at least one robot arm may include a base link 2710, at least one intermediate link 2720a, 2720b, 2720c, an instrument mounting link 2730, a yaw joint 2740, and at least one pitch joint 2750a, 2750b, 2750c, as illustrated in FIG. 26.

According to an aspect, the base link 2710 may be a link extending from the main body 2800 of the surgical robot. For example, as illustrated in FIG. 26, the base link 2710 may be extended from the main body 2800 of the surgical robot and may be rotatably coupled with the intermediate link 2720a, but is not limited thereto.

The instrument mounting link 2730 may be a link that is disposed at the end of the robot arm and to which a surgical instrument, which is inserted into a patient through a fixed remote center of motion (RCM) 2601, is mounted. As illustrated in FIG. 26, for example, the instrument mounting link 2730 may be provided with an instrument mounting unit on which a surgical instrument may be mounted. Such an instrument mounting unit may be configured to be slidable along a shaft axis direction of the surgical instrument, but is not limited thereto.

At least one intermediate link 2720a, 2720b, 2720c may be interposed between the base link 2710 and the instrument mounting link 2730. For example, as illustrated in FIG. 26, three intermediate links may be provided: a first intermediate link 2720a, a second intermediate link 2720b, and a third intermediate link 2720c. The base link 2710 and the first intermediate link 2720a may be connected, the first intermediate link 2720a and the second intermediate link 2720b may be connected, the second intermediate link 2720b and the third intermediate link 2720c may be connected, and the third intermediate link 2720c may be connected to the instrument mounting link 2730, but is not limited thereto.

According to an aspect, at least one yaw joint 2740 may be configured to provide a yaw direction rotational coupling of the robot arm between two links among the base link, the instrument mounting link, and the at least one intermediate link. As exemplarily illustrated in FIG. 26, the yaw joint 2740 may be coupled to allow the base link 2710 and the first intermediate link 2720a to rotate in the yaw direction of the robot arm, but is not limited thereto. For example, the first intermediate link 2720a may be configured to be rotatable about a yaw rotation axis 2741 of the robot arm with respect to the base link 2710. In an aspect, a yaw motor may be provided at the end of the base link 2710 to provide power for yaw direction rotation of the first intermediate link 2720a of the robot arm, but is not limited thereto, and the first intermediate link 2720a may be provided with a motor by itself and may be configured to perform yaw direction rotation of the robot arm. Hereinafter, the phrase “yaw direction of the robot arm” used herein refers to the yaw direction based on the robot arm, and may be distinguished from the phrase “yaw direction of the instrument” based on the surgical instrument. The description of the “yaw direction” for the robot arm should be understood to refer to the phrase “yaw direction of the robot arm”” unless otherwise stated, and the description of the “yaw direction” for the surgical instrument should be understood to refer to the phase “yaw direction of the instrument” unless otherwise stated.

According to an aspect, at least one pitch joint 2750a, 2750b, 2750c may be configured to provide a pitch direction rotational coupling of the robot arm between two links among the base link, the instrument mounting link, and the at least one intermediate link. As exemplarily illustrated in FIG. 26, at least one pitch joint may include, but is not limited to, three pitch joints: a first pitch joint 2750a, a second pitch joint 2750b, and a third pitch joint 2750c. As exemplarily illustrated in FIG. 26, the first pitch joint 2750a may be coupled to allow the first intermediate link 2720a and the second intermediate link 2720b to rotate in the pitch direction of the robot arm, the second pitch joint 2750b may be coupled to allow the second intermediate link 2720b and the third intermediate link 2720c to rotate in the pitch direction of the robot arm, and the third pitch joint 2750c may be coupled to allow the third intermediate link 2720c and the instrument mounting link 2730 to rotate in the pitch direction. However, it should be noted that such a configuration is merely exemplary and that the technical idea according to the present description is not limited thereto. Hereinafter, in this description, the phrase “pitch direction of the robot arm” refers to the pitch direction based on the robot arm, and may be distinguished from the phrase “pitch direction of the instrument” based on the surgical instrument. The description of the “pitch direction” for the robot arm should be understood to refer to the phrase “pitch direction of the robot arm” unless otherwise stated, and the description of the “pitch direction” for the surgical instrument should be understood to refer to the “pitch direction of the instrument” unless otherwise stated.

For example, as illustrated in FIG. 26, the instrument mounting link 2730 may be configured to be rotatable about the third pitch axis 2751c with respect to the third intermediate link 2720c. The third intermediate link 2720c may be configured to be rotatable about the second pitch axis 2751b with respect to the second intermediate link 2720b. The second intermediate link 2720b may be configured to be rotatable about the first pitch axis 2751a with respect to the first intermediate link 2720a.

According to an aspect, a pitch motor configured to provide power for pitch direction rotation of at least one intermediate link may be provided in at least one pitch joint among at least one pitch joint 2750a, 2750b, 2750c. Such a pitch motor may be provided in any one of the two links that are rotationally coupled by at least one pitch joint 2750a, 2750b, 2750c, but is not limited thereto. In addition, as described later in the present disclosure, each of at least one pitch joints 2750a, 2750b, 2750c may be configured such that the rotation of one of the pitch joints is linked to the rotation of the other pitch joint. Herein, only one of the at least one pitch joint 2750a, 2750b, 2750c may be provided with a pitch motor, and the power for the rotation of the link of the remaining pitch joint may be performed by the power of the pitch motor provided in the other pitch joint.

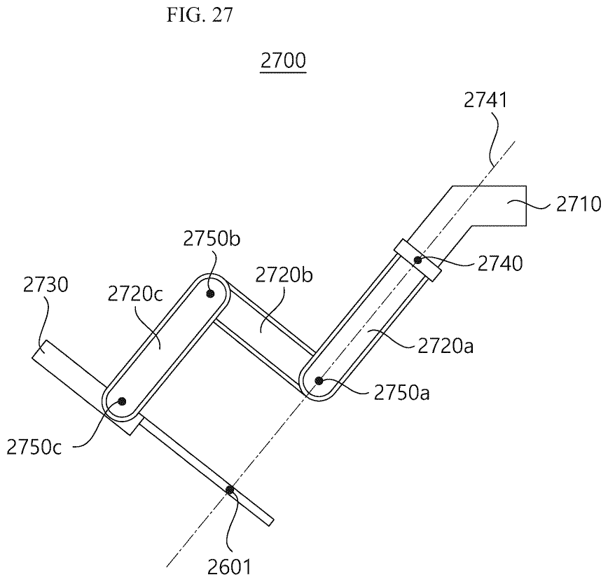

FIG. 27 is a side view illustrating a yaw rotation axis of a robot arm of the surgical robot system of FIG. 26. As illustrated in FIG. 27, according to an aspect of the present disclosure, the yaw rotation axis 2741 by the yaw joint 2740 may be configured to intersect the RCM 2601. Accordingly, even when the first intermediate link 2720a and the remaining links coupled thereto and the surgical instrument rotate about the yaw rotation axis 2741 as illustrated in FIGS. 26 and 27, the RCM 2601 may still be maintained. Accordingly, the surgical instrument always maintains a state of being inserted into the inside of the body of a patient through a port formed in the body of the patient, regardless of rotation in the yaw direction, and does not cause damage to the body of the patient.

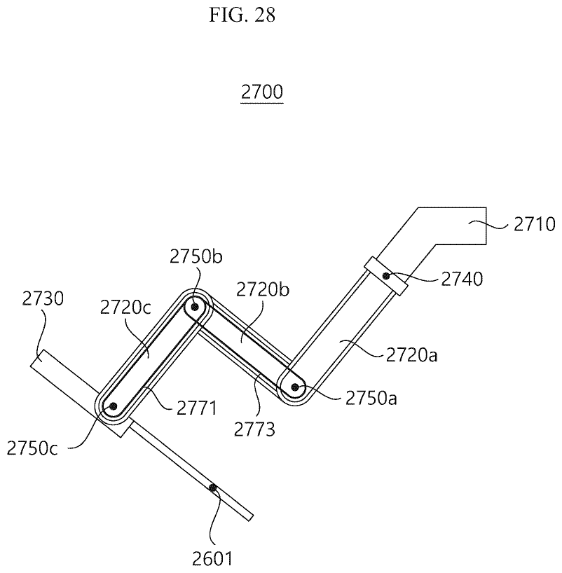

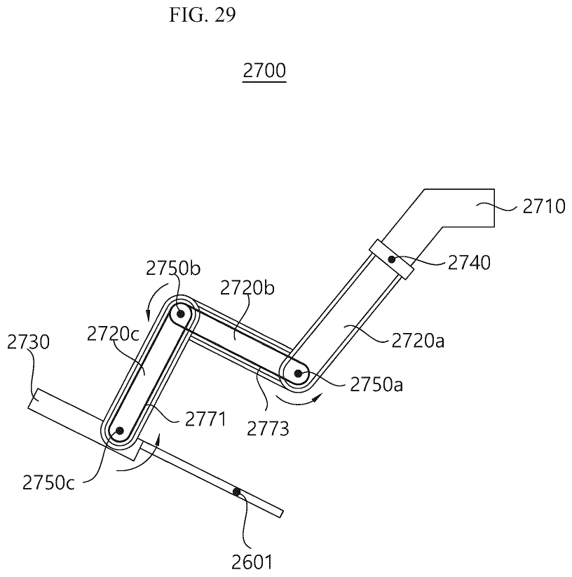

FIG. 28 is a side view illustrating rotational linkage of pitch joints of a robot arm of the surgical robot system of FIG. 26. FIG. 29 is a side view illustrating maintenance of remote center of motion according to pitch control of the robot arm of FIG. 28.

As illustrated in FIG. 28, according to an aspect of the present disclosure, the robot arm 2700 may include the plurality of pitch joints 2750a, 2750b, 2750c, and the rotations of the plurality of pitch joints may be configured to be interlinked with each other. For example, the rotation of the first pitch joint 2750a may be interlinked with the rotation of the second pitch joint 2750b, and the rotation of the second pitch joint 2750b may be interlinked with the rotation of the third pitch joint 2750c. Accordingly, when the second intermediate link 2720b performs a pitch direction rotation with respect to the first intermediate link 2720a, the third intermediate link 2720c may perform pitch direction rotation with respect to the second intermediate link 2720b. In addition, when the third intermediate link 2720c performs pitch direction rotation with respect to the second intermediate link 2720b, the instrument mounting link 2730 may perform pitch direction rotation with respect to the third intermediate link 2720c. As illustrated in FIG. 29, since rotations of the plurality of pitch joints 2750a, 2750b, 2750c are interlinked, even when at least one link provided in the robot arm 2700 performs pitch direction rotation, the RCM 2601 may be maintained in a fixed state. Accordingly, the surgical instrument always maintains a state of being inserted into the inside of the body of a patient through the port formed in the body of the patient regardless of rotation in the pitch direction, and does not cause damage to the body of the patient.

As illustrated in FIG. 28, according to an aspect, the rotations of the plurality of pitch joints 2750a, 2750b, 2750c may be configured to be interlinked with each other by a fixed belt 2771, 2773 that interlinks the rotations of at least two pitch joints. For example, the first fixed belt 2771 may be connected to the second pitch joint 2750b and the third pitch joint 2750c, and may interlink the pitch direction rotations of the second pitch joint 2750b and the third pitch joint 2750c. In addition, for example, the second fixed belt 2773 may be connected to the first pitch joint 2750a and the second pitch joint 2750b, and may interlink the pitch direction rotations of the first pitch joint 2750a and the second pitch joint 2750b. As a non-limiting example, the fixed belts 2771, 2773 may be belts made of a metal material, but are not limited thereto. It should be noted that any structure for interlinking the rotations of two axes may be adopted as a structure for interlinking the rotations of the pitch joint according to the embodiments of the present disclosure.

As described above, according to an embodiment of the present disclosure, the RCM may be fixed regardless of the roll direction rotation of the robot arm by configuring the roll direction rotation axis of the roll joint to intersect with the RCM 2601, and the RCM may be fixed regardless of the pitch direction rotation of the robot arm by interlinking the pitch direction rotations of the plurality of pitch joints. In other words, the surgical robot according to an embodiment of the present disclosure may be configured so that the RCM 2601 is fixed regardless of the roll direction and/or pitch direction rotation of the plurality of joints provided in the robot arm.

### Elastic Joint

When a surgery is performed using the surgical robot system, the robot arm performs the surgery under the control of a surgeon. According to embodiments of the present disclosure, as described above, it may be configured to operate while maintaining the RCM regardless of the roll direction and/or pitch direction rotation of the plurality of joints. However, when the robot arm and/or surgical instrument of the surgical robot collides with a specific object, the RCM may be deviated due to the collision, which may cause damage to the body of a patient.

In other words, when a robotic laparoscopic surgery is performed, the surgical robot arm performs work inside the body of a patient by the manipulation of a doctor. In this connection, structures for operating the surgical robot exist not only inside the body of the patient but also outside the body of the patient. Due to the nature of the surgery, there may be cases where movements that can cause collisions between structures outside the body are unavoidable. In such cases, the RCM of the surgical robot may be deviated due to the collision, and thus the robot arm may damage the skin on the abdomen of the patient. According to an aspect of the present disclosure, by utilizing the elastic joint, the robot arm may be controlled to detect and comply with an external force. Accordingly, it is possible to detect and avoid collisions, or to control movement in a direction that maintains the remote center of motion even if a collision occurs. Accordingly, a safe surgical robot arm may be designed even when a mutual collision occurs.

In this regard, according to an aspect of the present disclosure, at least one joint among the plurality of joints including a yaw joint or a pitch joint included in the robot arm may be the elastic joint that allows twisting between links that are targets of rotational coupling due to an external force. Herein, the twisting by the elastic joint may be configured to be performed along the rotational direction by the yaw joint or the pitch joint. Accordingly, the robot arm may be configured such that, by providing the elastic joint, when an external force is applied to the robot arm, twisting occurs in at least one of the yaw joint or the pitch joint along the rotational direction, so that the surgical instrument does not deviate from the RCM.

As a non-limiting example, the elastic joint may be configured as an elastic actuator in which a pitch and/or a yaw motor is connected in series with a motor, a spring, and an output axis. Such an elastic actuator may absorb an external force by twisting of a spring between the motor and the output axis, and may also measure the magnitude of the external force through the angular difference between the motor-side encoder and the output-side encoder and the spring coefficient of the spring. By absorbing the external force, the force generated by the collision between the surgical robots is converted into the twisting of the spring, which may prevent the RCM from changing through the fact that ‘even if the pitch and yaw motors rotate, the RCM remains unchanged’ described above in the present disclosure. Accordingly, the robot arm according to an embodiment of the present disclosure may respond to the collision and the external force.

For example, according to an aspect of the present disclosure, the yaw joint 2740 that rotatably connects the base link 2710 and the first intermediate link 2720a in the yaw direction may be the elastic joint that allows the twisting between the first intermediate link 2720a and the base link 2710 when an external force is applied to the robot arm. Herein, the elastic joint may be configured so that the first intermediate link 2720a and the base link 2710 are allowed to twist along a yaw joint rotational direction. The yaw joint rotational direction may represent the rotation in the direction of the yaw rotation axis 2741 that is rotated by the yaw motor. In other words, the yaw joint 2740 according to an aspect may be configured such that, in addition to allowing the first intermediate link 2720a to perform rotation about the yaw axis by driving the yaw motor of the yaw joint under the control of a processor, the first intermediate link 2720a is also allowed to rotate about the yaw axis by an external force when the external force is applied to the robot arm. Such rotation and/or twisting by the external force may be a separate movement from the rotation by the driving of the motor. By implementing the yaw joint as the elastic joint as such, when an external force is applied to the robot arm, the robot arm may be made to rotate in a yaw joint direction instead of moving or vibrating in an arbitrary direction. Accordingly, it is possible to prevent the surgical instrument from being deviated from the RCM and causing damage to the body of a patient.

Similarly, according to an aspect of the present disclosure, at least one of the pitch joints 2750a, 2750b, 2750c may be the elastic joint. For example, the first pitch joint 2750a may be the elastic joint. Accordingly, the first pitch joint (2750a) may be configured such that, in addition to allowing the second intermediate link (2720b) to perform rotation about the pitch axis by driving the pitch motor of the pitch joint under the control of a processor, the second intermediate link (2720b) is also allowed to rotate about the pitch axis by an external force when the external force is applied to the robot arm. Such rotation and/or twist due to the external force may be a separate movement from the rotation due to the driving of the motor. As described above, the plurality of pitch joints 2750a, 2750b, 2750c may be configured such that their rotations are linked to each other. Accordingly, for example, in the case where the first pitch joint 2750a is implemented as the elastic joint, when an external force is applied to the robot arm, instead of the robot arm moving or vibrating in an arbitrary direction, the interlinked pitch joints may be made to rotate about the pitch axis. Accordingly, it is possible to prevent the surgical instrument from being deviated from the RCM and causing damage to the body of a patient.

Although the case where the first pitch joint 2750a is the elastic joint has been described as an example, the scope of the technical idea of the present disclosure is not limited thereto. The second pitch joint or the third pitch joint may be configured as the elastic joint, or all pitch joints may be configured as the elastic joints. In addition, according to an aspect, in consideration of the rotational linkage between the pitch joints, only one of the pitch joints may be provided with a pitch motor to supply driving power for rotation, and the pitch joint provided with the pitch motor may also be configured as an elastic joint.

According to embodiments of the present disclosure, even when the direction of the external force is different from the rotational direction of the yaw joint and/or the pitch joint, instead of the robot arm moving or vibrating in the direction of the external force, at least some elements of the robot arm may be configured to rotate in the rotational direction of the yaw joint and/or the pitch joint. As a non-limiting example, the elastic modulus included in the elastic joint may be determined within a range in which, with respect to an external force, force components other than in the rotational direction of the yaw joint and/or the pitch joint are canceled, but force components in the rotational direction of the yaw joint and/or the pitch joint are accommodated so that twisting and/or rotation occurs.

FIG. 30 is an exemplary view of an elastic joint according to an aspect of the present disclosure. Referring to FIG. 30, an example of a non-limiting elastic joint is described in more detail.

The elastic joint according to an aspect of the present disclosure may be configured to couple a first link and a second link so as to allow mutual rotation. For example, when the yaw joint 2740 described above with reference to FIGS. 26 to 28 is the elastic joint, the first link may be the base link 2710 and the second link may be the first intermediate link 2720a. In addition, for example, when the first pitch joint 2750a described above is the elastic joint, the first link may be the first intermediate link 2720a and the second link may be the second intermediate link 2720b.

As illustrated in FIG. 30, the elastic joint according to an aspect of the present disclosure may include a motor 3010 coupled to the first link that is a target of rotational coupling of the elastic joint, and an elastic member 3030 whose first end is coupled to the motor and whose second end is coupled to a second link 3020 that is a target of rotational coupling of the elastic joint. Herein, the elastic member 3030 may provide a rotational direction twist of the rotational coupling of the elastic joint.

For example, the elastic joint may provide power for rotation in the rotational coupling direction of the elastic joint by operating the motor 3010 under the control of the processor. A motor axis 3013 may rotate by the operation of the motor 3010, and the elastic member 3030 and the second link 3020 coupled thereto may rotate. Accordingly, the second link 3020 rotates relative to the first link comprising the motor 3010 as much as the control command of the processor instructs.

Herein, an external force may be applied to the robot arm. For example, when an external force is applied to the second link 3020, the second link 3020 may rotate in the rotational direction of the elastic joint in relation to the first link comprising the motor 3010 due to the presence of the elastic member 3030. In other words, as a separate movement from the rotation according to the control command of the processor, the second link performs the rotation in the rotational direction of the elastic joint.

According to an aspect, the elastic member may be a coil spring surrounding a rotation axis 3031 of the rotational coupling provided by the elastic joint, but is not limited thereto. It is noted that any elastic member that causes the second link to rotate in the rotational direction of the elastic joint in response to the external force may be adopted.

According to an aspect of the present disclosure, the plurality of joints provided in the robot arm may be implemented as elastic joints, and elastic members provided in each elastic joint may be configured to have different elastic moduli. For example, an elastic yaw joint and an elastic pitch joint may be included in one robot arm, and elastic moduli of the elastic member of the elastic yaw joint and the elastic member of the elastic pitch joint may be different from each other. For example, the yaw joint may have a first elastic member having a first elastic modulus, and the pitch joint may have a second elastic member having a second elastic modulus. Herein, the second elastic modulus may be configured to be greater than the first elastic modulus. In contrast, the first elastic modulus may be configured to be greater than the second elastic modulus. When the first elastic modulus is set to be greater than the second elastic modulus, for example, when the plurality of robot arms are provided in one surgical robot as illustrated in FIG. 26, it may be configured such that rotation of the robot arm about the pitch axis in response to an external force is dominant over rotation of the robot arm about the yaw axis in response to an external force. Accordingly, the possibility of secondary collisions occurring between the robot arms due to rotation in the yaw axis direction may be minimized.

According to an aspect of the present disclosure, the plurality of robot arms provided in the surgical robot system may each include the elastic joint, and the elastic moduli of the elastic members of the elastic joints may be determined differently depending on the robot arms. For example, the surgical robot system may include a first robot arm and a second robot arm, and the elastic joint of the first robot arm may include the first elastic member, and the elastic joint of the second robot arm may include the second elastic member. Herein, the first elastic modulus of the first elastic member may be configured to be greater than the second elastic modulus of the second elastic member. In certain surgical situations, there may be cases where the surgical procedure needs to continue despite interference between the plurality of robot arms. In this connection, it is possible to set a specific robot arm to operate preferentially with respect to another robot arm by making the elastic members of the elastic joints of each robot arm have different elastic moduli. In other words, by configuring the elastic members of a primary robot arm to have a greater elastic modulus than the elastic members of a secondary robot arm, when the control of the two robot arms continues, the primary robot arm may reproduce the operation according to the control of the processor, while the secondary robot arm may receive an external force from the primary robot arm to cause the elastic joint to twist.

Referring again to FIG. 30, the elastic joint according to an aspect of the present disclosure may further include a first encoder 3015 for detecting a rotational position of the motor 3010 and a second encoder 3025 for detecting a rotational position of the second link 3020. Accordingly, information on the rotational position of the motor 3010 and information on the rotational position of the second link 3020 may be acquired. According to an aspect of the present disclosure, the processor provided in the surgical robot system may determine information on a rotation amount due to an external force of the elastic joint or information on the magnitude of the external force based on the information on the rotational position of the motor and the information on the rotational position of the second link. Thus, the processor may be configured to perform compliance control of the robot arm with respect to the external force, stop the control, or output warning information to a user. Hereinafter, procedures performed by the processor according to an embodiment of the present disclosure will be described in more detail.

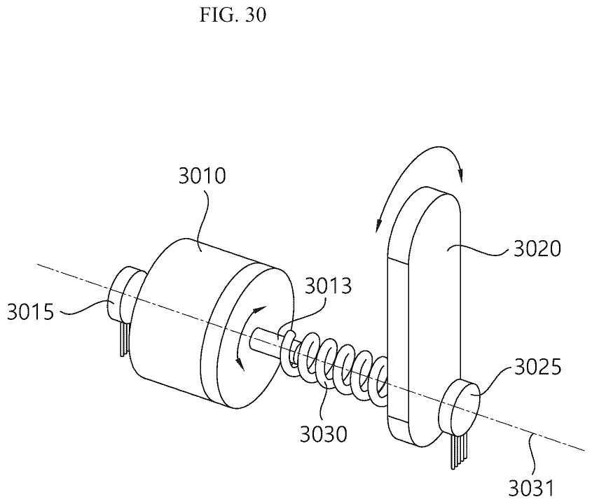

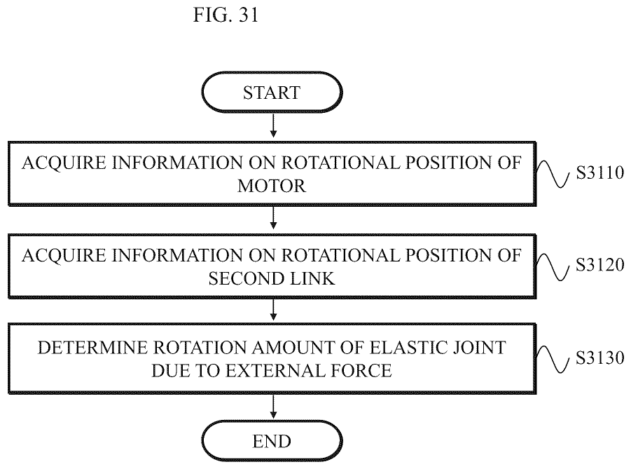

FIG. 31 is an exemplary flowchart of a rotation amount determination procedure for a robot arm according to an embodiment of the present disclosure. As illustrated in FIG. 31, the processor included in the surgical robot system may first acquire information on the rotational position of the motor (stage 3110). According to an aspect, the processor may acquire information on the rotational position of the motor based on the first encoder 3015 disposed near the motor 3010. Thereafter, the processor may acquire information on the rotational position of the second link 3020 (stage 3120). According to an aspect, the processor may acquire information on the rotational position of the second link 3020 based on the second encoder 3025 disposed near the second link 3020. Since the rotation of the second link is performed by the rotation of the motor unless an external force is applied to the robot arm having the elastic joint, the rotational position of the motor 3010 and the rotational position of the second link may be determined to be the same. When a reduction ratio or an acceleration ratio is applied to the rotation of the motor and the rotation of the second link according to an aspect, the processor may reflect the same, compare the rotational position of the motor with the rotational position of the second link, and determine whether the second link has rotated to an intended degree according to the control of the motor. Referring again to FIG. 31, the processor may be configured to determine the rotation amount of the elastic joint due to the external force based on the information on the rotational position of the motor and the information on the rotational position of the second link (stage 3130). For example, based on the information on the rotation amount of the second link intended by the control of the motor, the processor may determine the rotation amount exceeding or falling short of the intended rotation amount of the second link as the rotation amount of the elastic joint due to the external force. In other words, it may be determined how much the second link has rotated and in which direction the second link has rotated due to an external force applied to the robotic arm.

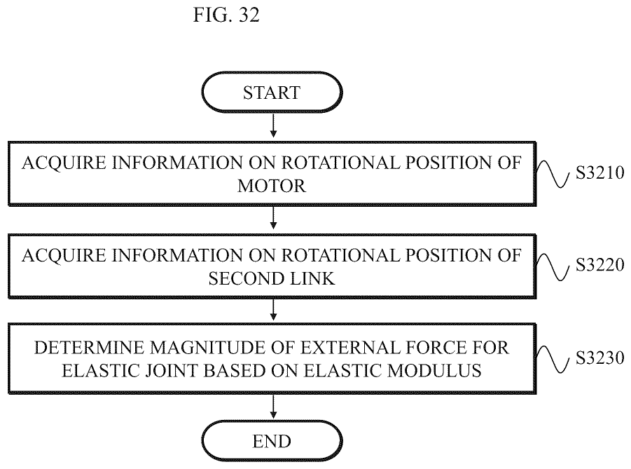

FIG. 32 is an exemplary flowchart of an external force magnitude determination procedure for a robot arm according to an embodiment of the present disclosure. As illustrated in FIG. 32, the processor included in the surgical robot system may first acquire information on the rotational position of the motor (stage 3210). According to an aspect, the processor may acquire information on the rotational position of the motor based on the first encoder 3015 disposed near the motor 3010. Thereafter, the processor may acquire information on the rotational position of the second link 3020 (stage 3220). According to an aspect, the processor may acquire information on the rotational position of the second link 3020 based on the second encoder 3025 disposed near the second link 3020. Since the rotation of the second link is performed by the rotation of the motor unless an external force is applied to the robot arm having the elastic joint, the rotational position of the motor 3010 and the rotational position of the second link may be determined to be the same. Thereafter, the processor may be configured to determine the magnitude of the external force applied to the robot arm at a first time point based on the information on the rotational position of the motor, the information on the rotational position of the second link, and the elastic modulus of the elastic member 3030 provided in the elastic joint. As described above, the extent to which the second link has rotated due to anexternal force may be identified by comparing the rotational position of the motor and the rotational position of the second link, and information on the magnitude of the external force applied to the elastic joint may be determined using the elastic modulus of the elastic member 3030 and the rotation amount of the elastic joint due to the external force. For example, when an elastic member having a greater elastic modulus is used, it may be determined that a greater external force is applied for the same rotation amount of the elastic joint compared to when an elastic member having a smaller elastic modulus is used. The specific computational mechanism for measuring the external force is not limited to a specific method. The magnitude of the external force determined herein may include, for example, information on the torque applied to the elastic joint. By determining the information on the torque applied to the elastic joint as such, according to an embodiment of the present disclosure, an advantageous effect is achieved in that the information on the torque applied to the robot arm may be determined without providing an expensive torque sensor to the robot arm.

According to the embodiments of the present disclosure, it is possible to control the robot arm that allows the occurrence of a collision by using the measured information on the magnitude of the external force applied to the robot arm and/or the rotation amount due to the external force. Hereinafter, for the convenience of explanation, procedures for comparing the magnitude of the external force with a threshold value are described, but the technical idea of the present disclosure is not limited thereto, and it should be understood that the robot arm may be controlled by comparing the rotation amount of the elastic joint due to the external force or other measured values with the threshold value.

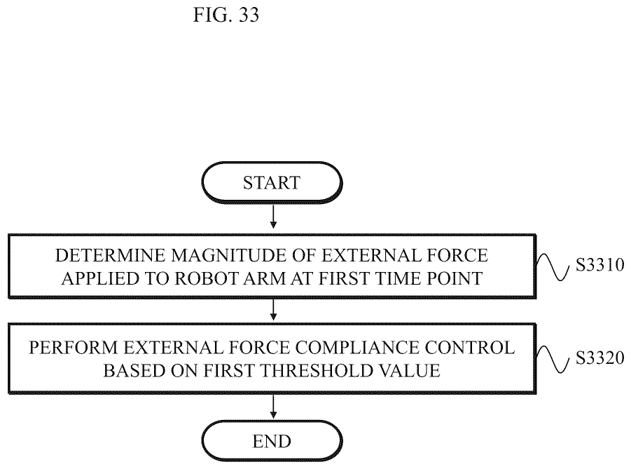

FIG. 33 is an exemplary flowchart of an external force compliance control procedure for a robot arm according to an embodiment of the present disclosure. As illustrated in FIG. 33, the processor included in the surgical robot system may first determine the magnitude of the external force applied to the robot arm at the first time point (stage 3310). The magnitude of the external force applied to the robot arm at the first time point may be determined by, for example, the procedure described above with reference to FIG. 32, but it should be noted that it is not limited thereto. For example, the determination of the magnitude of the external force applied to the robot arm by providing a torque sensor may also be included in the technical idea of the present disclosure.

Thereafter, the processor may be configured to control the motor of the elastic joint so that the elastic joint complies with the external force, in response to a determination that the magnitude of the determined external force at the first time point is greater than or equal to a predetermined first threshold value. For example, the processor may maintain control of the robot arm according to the manipulation of a user until the external force applied to the elastic joint reaches the first threshold value. In other words, the surgical robot system according to an aspect of the present disclosure may maintain control of the robot arm even in a situation where a collision or conflict occurs with the robot arm and an external force is applied to the robot arm. This may be allowed by maintaining the RCM by ensuring that the robot arm does not move or vibrate in an unintended direction even when an external force is applied as described above, and rotates in the rotational direction of the joint by at least one elastic joint. However, when the magnitude of the external force at the first time point exceeds the first threshold value, the processor may be configured to control the motor of the elastic joint to comply with the external force, rather than controlling the robot arm while resisting the external force any longer. In other words, the processor may control the motor in the direction of reducing the external force, unlike the motor control corresponding to the manipulation of a user. Accordingly, the twisting due to the elastic member of the elastic joint is resolved, and the robot arm may enter a state in which no external force is applied.

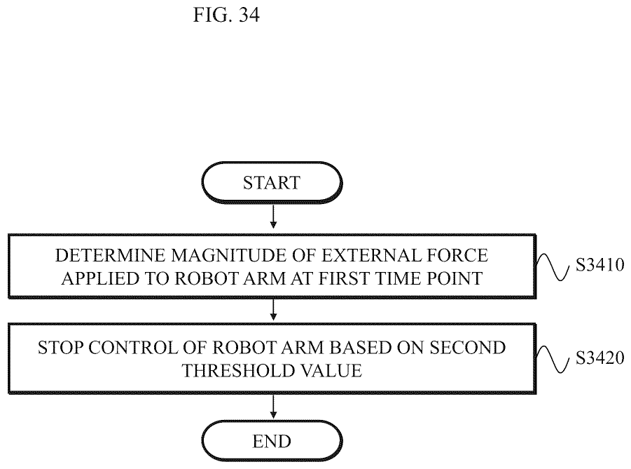

FIG. 34 is an exemplary flowchart of a control interruption procedure for a robot arm according to an embodiment of the present disclosure. As illustrated in FIG. 34, the processor included in the surgical robot system may first determine the magnitude of the external force applied to the robot arm at the first time point (stage 3410). The magnitude of the external force applied to the robot arm at the first time point may be determined by, for example, the procedure described above with reference to FIG. 32, but it should be noted that it is not limited thereto. For example, the determination of the magnitude of the external force applied to the robot arm by providing the torque sensor may also be included in the technical idea of the present disclosure.

Thereafter, the processor may be configured to stop the control of the robot arm, in response to a determination that the magnitude of the determined external force at the first time point is greater than or equal to a predetermined second threshold value (stage 3420). In other words, when the processor determines that the external force acting on the elastic joint is excessively large, the processor may be configured to stop the control of the entire surgical robot system or the robot arm where the external force is applied or the corresponding elastic joint for reasons such as preventing damage to the robot arm or preventing a safety accident.

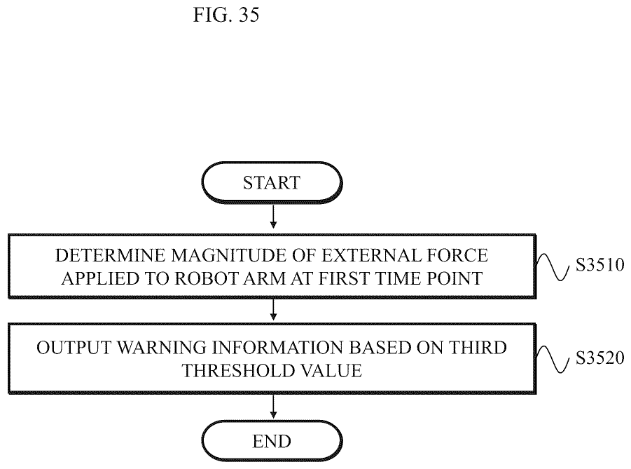

FIG. 35 is an exemplary flowchart of a warning information output procedure for a robot arm according to an embodiment of the present disclosure. As illustrated in FIG. 35, the processor included in the surgical robot system may first determine the magnitude of the external force applied to the robot arm at the first time point (stage 3510). The magnitude of the external force applied to the robot arm at the first time point may be determined by, for example, the procedure described above with reference to FIG. 32, but it should be noted that it is not limited thereto. For example, the determination of the magnitude of the external force applied to the robot arm by providing the torque sensor may also be included in the technical idea of the present disclosure.

Thereafter, the processor may be configured to output warning information through the output interface provided in the surgical robot system, in response to a determination that the determined magnitude of the external force at the first time point is greater than or equal to a predetermined third threshold value (stage 3520). For example, the processor may output a warning sound or warning guidance through a speaker provided in the surgical robot system, or may display a warning message through a display provided in the surgical robot system. In addition, the warning information may be output through a haptic feedback device provided in the user input interface. It should be noted that the form and output device of the warning information are not limited to a specific mean. According to an aspect of the present disclosure, by delivering the fact that the external force acting on the surgical joint exceeds a certain level to the user, it is possible to cause the user to change the degree of control or to recognize and respond to whether a safety accident has occurred or whether a failure has occurred.

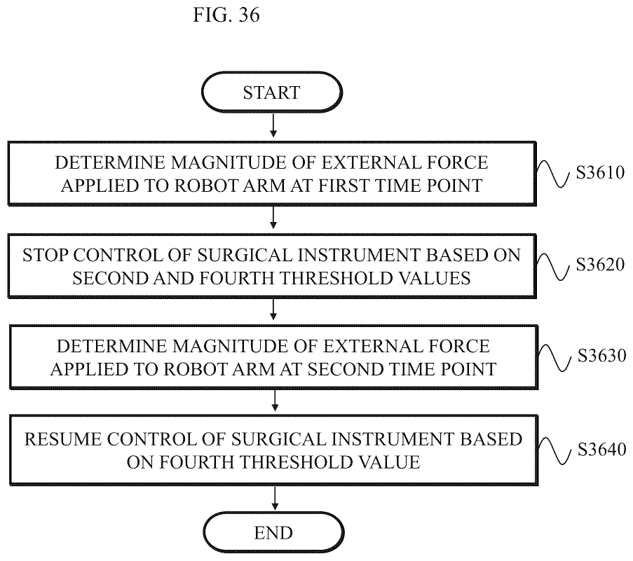

FIG. 36 is an exemplary flowchart of a control interruption and resumption procedure for a robot arm and/or a surgical instrument according to an embodiment of the present disclosure. As illustrated in FIG. 36, the processor included in the surgical robot system may first determine the magnitude of the external force applied to the robot arm at the first time point (stage 3510). The magnitude of the external force applied to the robot arm at the first time point may be determined by, for example, a procedure as described above with reference to FIG. 32, but it should be noted that it is not limited thereto. For example, the determination of the magnitude of the external force applied to the robot arm by providing the torque sensor may also be included in the technical idea of the present disclosure.

Thereafter, the processor may be configured to stop control of the surgical instrument and maintain control of the robot arm, in response to a determination that the determined magnitude of the external force at the first time point is greater than or equal to a predetermined fourth threshold value and less than a second threshold value (stage 3620). In other words, when the magnitude of the external force acting on the surgical robot arm and/or the elastic joint is within a certain range, the processor may stop control of the surgical instrument to prevent unintended contact or damage caused by the instrument operation that may occur inside the body of a patient, while maintaining control of the robot arm. The fourth threshold value for the external force may be the magnitude of the external force that is deemed dangerous for the operation of the surgical instrument, but is not limited thereto. The second threshold value may be the magnitude of the external force that is deemed dangerous for the operation of the surgical robot arm, similar to the aforementioned embodiment, but is not limited thereto.

Referring again to FIG. 36, the processor may determine the magnitude of the external force applied to the robot arm at a second time point subsequent to the first time point. In other words, the magnitude of the external force applied to the robot arm after a certain amount of time has elapsed may be determined again. It should be noted that the magnitude of the external force applied to the robot arm at the second time point may be determined by, for example, the procedure described above with reference to FIG. 32, but is not limited thereto. For example, the determination of the magnitude of the external force applied to the robot arm by providing the torque sensor may also be included in the technical idea of the present disclosure.

The processor may be configured to resume control of the surgical instrument, in response to a determination that the determined magnitude of the external force at the second time point is less than the fourth threshold value. The external force on the surgical robot arm and/or elastic joint may be removed by various non-limiting factors, such as natural removal of the external force or a change in control according to the perception of the external force by a user. After the magnitude of the applied external force is reduced to less than the fourth threshold value, the processor may resume control of the surgical instrument that was stopped, so that an actual surgical procedure may be performed inside the body of a patient.

Through such control, even if an external force is present, control of the robot arm may be maintained to achieve movement to a desired position and/or a change in posture, while control of the surgical instrument, which directly affects the inside of the body, is permitted only after the external force has been reduced to or below a predetermined level, thereby enabling a safer surgical procedure.

The procedures according to an embodiment of the present disclosure as described above may be composed of stages that are processed sequentially over time, for example, in the user terminal 2000, 2010 or the processor 2011 illustrated in FIGS. 1 and 2A. In addition, as described above with reference to FIGS. 1 and 2B, at least one of the stages of the procedures described above may be processed by the servers 3000, 301 or the processor 3011.

In addition, as described above with reference to FIGS. 3 to 5, at least one of the stages of the procedures described above may be processed by the master robot 10, the slave robot 20, the surgical instrument 30, or a processor included therein.

Hereinbefore, for the convenience of explanation, although the driving element provided in the surgical robot system has been described with reference to a non-limiting example in which the driving element is a robot arm, the technical idea of the present disclosure is not limited thereto, as described above,. It should be understood that the driving element provided in the surgical robot system includes all arbitrary configurations that generate movement through a connection relationship between a specific member and another member.

### Slide Driving Unit

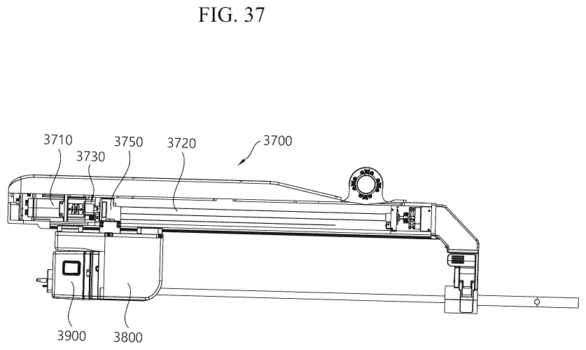

FIG. 37 is a perspective view of a slide driving unit having an elastic joint member according to an embodiment of the present disclosure. Hereinafter, referring to FIG. 37, the slide driving unit having the elastic joint member according to an aspect of the present disclosure will be described more specifically, but not limitedly.

As described above, the surgical robot system according to an aspect of the present disclosure includes the at least one driving element having the elastic joint and the processor configured to control the same. According to an aspect, the surgical robot system includes the at least one driving element, wherein the driving element may include the first member, the second member coupled so as to be capable of changing its relative position with respect to a first member, and the at least one joint member providing a rotational coupling between the first member and the second member. Herein, the at least one joint member may be the elastic joint member that allows twisting between members that are targets of rotational coupling due to an external force.

In this regard, for example, as illustrated in FIG. 37, the driving element may include a slide driving unit 3700 that controls a linear movement of an instrument mounting unit 3800 in a patient insertion direction. Herein, the first member may be a slide motor 3710 provided in the slide driving unit, and the second member may be a slide shaft 3720 provided in the slide driving unit. According to an aspect, the joint member may include an elastic member 3730 disposed between the slide motor 3710 and the slide shaft 3720.

According to an aspect, the instrument mounting unit 3800 may be configured to move linearly along an extension direction of the instrument mounting link 2730 mentioned above, but is not limited thereto. As described above, the instrument mounting link 2730 may be, for example, a link disposed at the end of the robot arm, on which the surgical instrument inserted into a patient through a fixed remote center of motion (RCM) 2601 is mounted, but is not limited thereto. In addition, according to an aspect, the instrument mounting unit 3800 may be configured to be slidably moved along the shaft axis direction of the surgical instrument, but is not limited thereto.

As exemplarily illustrated in FIG. 37, a member for guiding a sliding of the instrument mounting unit 3800 may be disposed on an upper portion of the instrument mounting link. The instrument mounting unit 3800 may be slidably moved along such a member. As an example, power for the sliding of the instrument mounting unit 3800 may be provided from the instrument mounting link. For example, as exemplarily illustrated in FIG. 37 in this description, a ball screw structure may be adopted to convert the rotation of the slide shaft 3720 according to the rotation of the slide motor 3710 into a straight motion, but is not limited thereto. A slide moving member 3750 by the ball screw may be configured to move linearly along a parallel movement path in the shaft extension direction, and may be coupled with the instrument mounting unit 3800 to cause the instrument mounting unit 3800 to slide.

In this exemplary slide driving unit 3700, since the slide shaft 3720 is required to be rotated by the slide motor 3710 to provide linear movement to the instrument mounting unit 3800, according to an aspect, an elastic member may be provided between the slide motor 3710 and the slide shaft 3720. The position of the elastic member 3730 illustrated in FIG. 37 is merely an example for explanation, and the position of the elastic member is not limited thereto. By providing the elastic member as such, particularly, for example, even when at least one of the instrument mounting unit 3800 or the surgical instrument or its driving unit 3900 collides with an obstacle, the impact may be absorbed by the elastic member and the impact on the body may be minimized.

### Driving Unit and Mounting Unit of Surgical Instrument

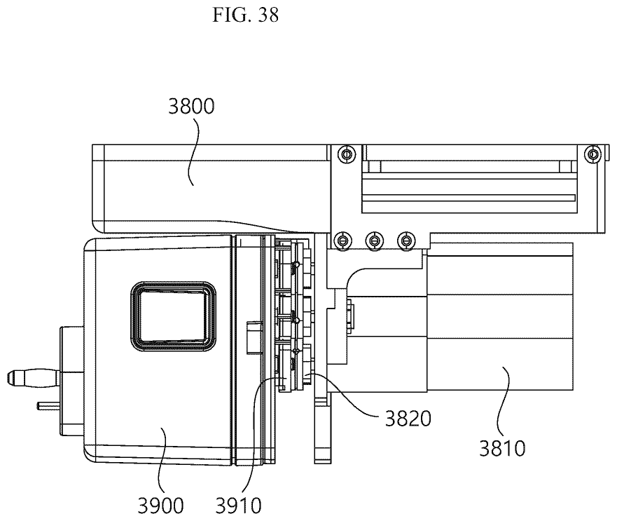

FIG. 38 is a perspective view of a driving unit and/or an instrument mounting unit of a surgical instrument having an elastic joint member according to an embodiment of the present disclosure. Hereinafter, with reference to FIG. 38, a driving unit 3900 or the instrument mounting unit 3800 of the surgical instrument having the elastic joint member according to an aspect of the present disclosure will be described more specifically, but not limitedly.

As described above, the surgical robot system according to an aspect of the present disclosure includes the at least one driving element having the elastic joint and the processor configured to control the same. According to an aspect, the surgical robot system includes the at least one driving element, wherein the driving element may include the first member, the second member coupled so as to be capable of changing its relative position with respect to a first member, and the at least one joint member providing a rotational coupling between the first member and the second member. Herein, the at least one joint member may be the elastic joint member that allows twisting between members that are targets of rotational coupling due to an external force.

In this regard, as illustrated in FIG. 38, for example, the driving element may include the driving unit 3900 and the instrument mounting unit 3800 of the surgical instrument. Herein, the first member may be at least one pulley 3910 provided in the driving unit of the surgical instrument, and the second member may be an instrument driving motor 3810 provided in the instrument mounting unit 3800. According to an aspect, the joint member may include the elastic member (not shown) disposed between the at least one pulley 3910 and the instrument driving motor 3810.

According to an aspect, the instrument mounting unit 3800 may be configured to move linearly along the extension direction of the instrument mounting link 2730 mentioned above, but is not limited thereto. As described above, the instrument mounting link 2730 may be, for example, a link disposed at the end of the robot arm, on which a surgical instrument inserted into a patient through a fixed remote center of motion (RCM) 2601 is mounted, but is not limited thereto. As illustrated in FIG. 38, the instrument mounting unit 3800 may be configured to be provided on the robot arm so that the surgical instrument, more specifically, the driving unit 3900 of the surgical instrument, may be mounted thereon. Accordingly, the surgical instrument may be coupled to the robot arm through the instrument mounting unit 3800.

The instrument driving motor 3810 for providing power to control the operation of the surgical instrument may be provided in the instrument mounting unit 3800. Accordingly, for example, the instrument mounting unit 3800 may also be referred to as a ‘motor pack,’ but is not limited thereto. The torque of the instrument driving motor 3810 may be provided to the surgical instrument via, for example, a power providing pulley 3820 provided in the instrument mounting unit 3800 and a power receiving pulley 3910 provided in the instrument driving unit 3900 and fastened to the power providing pulley 3820. The rotation of the power receiving pulley 3910 may provide, for example, tension and/or pulling force to at least a wire provided in the surgical instrument, thereby controlling at least one of a jaw disposed on the opposite side of the wire, an instrument yaw axis, or an instrument pitch axis.

According to an aspect of the present disclosure, the elastic member may be disposed at any point between at least one pulley provided in the driving unit 3900 of an instrument, such as the power receiving pulley 3910, and the instrument driving motor 3810. Accordingly, the elastic member may absorb the impact of a collision occurring in at least a portion of at least one jaw provided in the surgical instrument, the instrument yaw axis, the instrument pitch axis, or a shaft provided in the surgical instrument, and may minimize the impact on the body. In such an exemplary slide driving unit 3700, since the slide shaft 3720 is required to be rotated by the slide motor 3710 in order to provide a linear movement to the instrument mounting unit 3800, according to an aspect, the elastic member may be provided between the slide motor 3710 and the slide shaft 3720. The position of the elastic member 3730 illustrated in FIG. 37 is merely an example for explanation, and the position of the elastic member is not limited thereto. By providing the elastic member as such, particularly, for example, even when at least one of the instrument mounting unit 3800 or the surgical instrument or its driving unit 3900 collides with an obstacle, the impact may be absorbed by the elastic member and the impact on the body may be minimized.

The method according to the present disclosure described above may be implemented as a computer-readable code on a computer-readable recording medium. The computer-readable recording medium includes any type of recording medium in which data that can be read by a computer system is stored, such as a read only memory (ROM), a random access memory (RAM), a magnetic tape, a magnetic disk, a flash memory, an optical data storing device, etc. Additionally, the computer-readable recording medium may be dispersed in the computer system connected by a computer communication network, and thus can be stored and executed as a code which can be read in a dispersed manner.

The aforementioned method may be included and provided in a computer program product. The computer program product may be traded between a seller and a buyer as a product. The computer program product may be distributed in the form of machine-readable storage medium (for example, a compact disc read only memory (CD-ROM)) or may be directly distributed (for example, download or upload) online through an application store (for example, a Play Store™) or between two user devices (for example, the smartphones). In the case of online distribution, at least a portion of the computer program product may be temporarily stored or generated in a machine-readable storage medium such as a memory of a manufacturer's server, an application store's server, or a relay server.

Although explained above with reference to the drawings or embodiments, it does not mean that the scope of protection of the present disclosure is limited by the drawings or embodiments, and it should be understood that a person skilled in the art can variously modify and change the present disclosure within a scope not deviating from the idea and area of the present disclosure as recited in the following claims.

Specifically, the characteristics explained may be executed in a digital electronic circuit, or a computer hardware, a firmware, or a combination thereof. The characteristics may be executed in a computer program product implemented within a storage device in a machine-readable storage device, for example, for execution by a programmable processor. Additionally, the characteristics may be performed by a programmable processor executing a program of instructions for performing functions of the explained embodiments by operating on the input data and generating the output. The explained characteristics may be executed within at least one computer programs which can be executed on a programmable system including at least one programmable processor, at least one input device, and at least one output device which are combined in order to receive data and instructions from the data storage system, and transmit data and instructions to the data storage system. The computer program includes a set of instructions which can be used directly or indirectly in a computer in order to perform a specific operation for a predetermined result. The computer program is written in any form of programming language including complied or integrated languages, and may be used in any form included as another unit suitable for use in a module, an element, a subroutine, or another computer environment, or as an independently-operating program.

Processors suitable for executing a program of instructions include, for example, both general and special purpose microprocessors, and either a single processor or multi-processors of different types of computers. Also, storage devices suitable for implementing computer program instructions and data embodying the explained characteristics include, for example, semiconductor memory devices such as EPROM, EEPROM, and flash memory devices, magnetic devices such as internal hard disks and removable disks, optical magnetic disks, and all types of non-volatile memory including CD-ROM and DVD-ROM disks. The processor and memory may be integrated in application-specific integrated circuits (ASIC) or added by the ASICs.

Although the above-mentioned present disclosure is explained based on a series of functional blocks, it is not limited by the aforementioned embodiments and attached drawings. Additionally, it would be obvious to a person skilled in the art to which the present disclosure pertains that various substitutions, modifications and changes are possible within a scope not deviating the technical idea of the present disclosure.

A combination of the above-mentioned embodiments is not limited to the aforementioned embodiments, and various types of combinations may be provided as well as the aforementioned embodiments according to implementation and/or necessity.

In the above-mentioned embodiments, the methods are explained based on a flow chart with a series of steps or blocks, but the present disclosure is not limited to the order of the steps, and some steps may be performed in a different order with other steps other than the above, or may be performed at the same time. Also, a person skilled in the art would understand that the steps in the flow chart are not exclusive, other steps can be included, or one or more steps in the flow chart can be deleted without affecting the scope of the present disclosure.

The above-mentioned embodiments include various aspects of examples. Although all possible combinations to express various aspects cannot be described, a person skilled in the art would recognize that other combinations are possible. Therefore, the present disclosure should include all other substitutions, modifications, and variations falling within the scope of the following claims.

What is claimed is:

1. A surgical robot system, comprising:
at least one driving element; and
a processor configured to control the driving element,
wherein the driving element comprises:
a first member;
a second member configured to change a relative position of the second member with respect to a first member; and
at least one joint member configured to provide a rotational coupling between the first member and the second member,
wherein the at least one joint member is an elastic joint member configured to allow twisting between the first member and the second member, which are targets of rotational coupling due to an external force.

2. The surgical robot system of claim 1, wherein the driving element comprises a robot arm,
wherein the surgical robot system comprises at least one surgical robot,
wherein the processor configured to control the surgical robot arm,
wherein the robot arm comprises:
a base link extending from a main body of the at least one surgical robot;
an instrument mounting link on which a surgical instrument to be inserted into a patient through a fixed remote center of motion (RCM) is mounted;
at least one intermediate link interposed between the base link and the instrument mounting link;
at least one yaw joint configured to provide a yaw direction rotational coupling of the robot arm between any two links among the base link, the instrument mounting link and the at least one intermediate link; and
at least one pitch joint configured to provide a pitch direction rotational coupling of the robot arm between any two links among the base link, the instrument mounting link and the at least one intermediate link,
wherein at least one of the at least one yaw joint and the at least one pitch joint is an elastic joint configured to allow twisting between links, which are the targets of rotational coupling due to the external force.

3. The surgical robot system of claim 2, wherein a yaw direction rotation axis of the robot arm by the yaw joint is configured to intersect the RCM.

4. The surgical robot system of claim 3, wherein:
the robot arm comprises a plurality of pitch joints, which include the at least one pitch joint; and
rotations of the plurality of pitch joints are configured to be interlinked with each other.

5. The surgical robot system of claim 4, wherein the rotations of the plurality of pitch joints are configured to be interlinked with each other by a fixed belt that interlinks the rotations of at least two pitch joints.

6. The surgical robot system of claim 4, wherein the twisting by the elastic joint is configured to be performed along a rotational direction by the at least one yaw joint or the at least one pitch joint.

7. The surgical robot system of claim 6, wherein the robot arm is configured such that, by providing the elastic joint, when the external force is applied to the robot arm, twisting occurs in at least one of the at least one yaw joint and the at least one pitch joint along the rotational direction, so that the surgical instrument does not deviate from the RCM.

8. The surgical robot system of claim 2, wherein the elastic joint comprises:
a motor coupled to a first link that is a target of the rotational coupling of the elastic joint; and
an elastic member whose first end is coupled to the motor and whose second end is coupled to a second link that is the target of the rotational coupling of the elastic joint, thereby providing twisting in the rotational direction of the rotational coupling of the elastic joint.

9. The surgical robot system of claim 8, wherein the elastic member is a coil spring that surrounds a rotation axis of the rotational coupling provided by the elastic joint.

10. The surgical robot system of claim 8, wherein the elastic joint further comprises:
a first encoder configured to detect a rotational position of the motor; and
a second encoder configured to detect a rotational position of the second link.

11. The surgical robot system of claim 10, wherein the processor is further configured to determine a rotation amount of the elastic joint due to the external force, based on information on the rotational position of the motor and information on the rotational position of the second link.

12. The surgical robot system of claim 10, wherein the processor is further configured to determine a magnitude of the external force applied to the robot arm at a first time point, based on information on the rotational position of the motor, information on the rotational position of the second link, and an elastic modulus of the elastic member.

13. The surgical robot system of claim 12, wherein the magnitude of the external force comprises information on a torque applied to the elastic joint.

14. The surgical robot system of claim 12, wherein the processor is further configured to control the motor of the elastic joint so that the elastic joint complies with the external force, in response to a determination that the determined magnitude of the external force at the first time point is greater than or equal to a predetermined first threshold value.

15. The surgical robot system of claim 12, wherein the processor is further configured to stop controlling of the robot arm, in response to a determination that the determined magnitude of the external force at the first time point is greater than or equal to a predetermined second threshold value.

16. The surgical robot system of claim 12, wherein the processor is further configured to output warning information through an output interface provided in the surgical robot system, in response to a determination that the determined magnitude of the external force at the first time point is greater than or equal to a predetermined third threshold value.

17. The surgical robot system of claim 12, wherein the processor is further configured to stop controlling of the surgical instrument and maintain controlling of the robot arm, in response to a determination that the determined magnitude of the external force at the first time point is greater than or equal to a predetermined fourth threshold value and less than a predetermined second threshold value.

18. The surgical robot system of claim 17, wherein the processor is further configured to determine the magnitude of the external force applied to the robot arm at a second time point subsequent to the first time point, and to resume controlling of the surgical instrument, in response to a determination that the magnitude of the external force at the second time point is less than the fourth threshold value.

19. The surgical robot system of claim 1, wherein:
the driving element comprises a slide driving unit configured to control linear motion of an instrument mounting unit in a patient insertion direction;
the first member comprises a slide motor provided in the slide driving unit;
the second member comprises a slide shaft provided in the slide driving unit; and
the joint member comprises an elastic member disposed between the slide motor and the slide shaft.

20. The surgical robot system of claim 1, wherein:
the driving element comprises a driving unit of a surgical instrument and an instrument mounting unit;
the first member comprises at least one pulley provided in the driving unit of the surgical instrument;
the second member comprises an instrument driving motor provided in the instrument mounting unit; and
the joint member comprises an elastic member disposed between the at least one pulley and the instrument driving motor.
